SEQUENCE LISTING (113 sequences)

SEQ ID NO: 1 - DNA, 66 bp, Homo sapiens
  source 1..66: mol_type = other DNA
atgggagccgcccgcctgctgcccaacctcactctgtgcttacagctgctgattctctgctgtcaa

SEQ ID NO: 2 - DNA, 54 bp, Homo sapiens
  source 1..54: mol_type = other DNA
atggggctggcctggggactaggcgtcctgttcctgatgcatgtgtgtggcacc

SEQ ID NO: 3 - DNA, 72 bp, Homo sapiens
  source 1..72: mol_type = other DNA
atgggaatcccaatggggaagtcgatgctggtgcttctcaccttcttggccttcgcctcgtgctgcattgct

SEQ ID NO: 4 - DNA, 87 bp, Homo sapiens
  source 1..87: mol_type = other DNA
atgcacgtgcgctcactgcgagctgcggcgccgcacagcttcgtggcgctctgggcacccctgttcctgctgcgctccgccctggcc

SEQ ID NO: 5 - DNA, 54 bp, Homo sapiens
  source 1..54: mol_type = other DNA
atgaagtgggtaacctttatttcccttctttttctctttagctcggcttattcc

SEQ ID NO: 6 - DNA, 57 bp, Homo sapiens
  source 1..57: mol_type = other DNA
atgacccggctgacagtcctggccctgctggctggtctgctggcgtcctcgagggcc

SEQ ID NO: 7 - DNA, 54 bp, Homo sapiens
  source 1..54: mol_type = other DNA
atgagggcctggatcttctttctcctttgcctggccgggagggctctggcagca

SEQ ID NO: 8 - DNA, 51 bp, synthetic construct
  misc_feature 1..51: note = Gaussia princeps signal sequence for luciferase
  source 1..51: mol_type = other DNA
atgggagtcaaagttctgtttgccctgatctgcatcgctgtggccgaggcc

SEQ ID NO: 9 - protein, 22 aa, Homo sapiens
MGAARLLPNLTLCLQLLILCCQ

SEQ ID NO: 10 - protein, 18 aa, Homo sapiens
MGLAWGLGVLFLMHVCGT

SEQ ID NO: 11 - protein, 24 aa, Homo sapiens
MGIPMGKSMLVLLTFLAFASCCIA

SEQ ID NO: 12 - protein, 29 aa, Homo sapiens
MHVRSLRAAAPHSFVALWAPLFLLRSALA

SEQ ID NO: 13 - protein, 18 aa, Homo sapiens
MKWVTFISLLFLFSSAYS

SEQ ID NO: 14 - protein, 19 aa, Homo sapiens
MTRLTVLALLAGLLASSRA

SEQ ID NO: 15 - protein, 18 aa, Homo sapiens
MRAWIFFLLCLAGRALAA

SEQ ID NO: 16 - protein, 17 aa, synthetic construct
  REGION 1..17: note = Gaussia princeps signal sequence for luciferase
MGVKVLFALICIAVAEA

SEQ ID NO: 17 - DNA, 582 bp, Homo sapiens
  source 1..582: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacg

SEQ ID NO: 18 - DNA, 543 bp, synthetic construct
  misc_feature 1..543: note = FGF17 deletion mutant
  source 1..543: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacc

SEQ ID NO: 19 - DNA, 474 bp, synthetic construct
  misc_feature 1..474: note = FGF17 deletion mutant
  source 1..474: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaa

SEQ ID NO: 20 - DNA, 582 bp, synthetic construct
  misc_feature 1..582: note = FGF17 mutant
  source 1..582: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccagcggacccagcgcacacggcggccccagcccctcacg

SEQ ID NO: 21 - DNA, 522 bp, synthetic construct
  misc_feature 1..522: note = FGF17 deletion mutant
  source 1..522: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgag

SEQ ID NO: 22 - DNA, 582 bp, synthetic construct
  misc_feature 1..582: note = FGF17 mutant
  source 1..582: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgaggctcaggcacagttcgagtttgtgggcgctgcccccacccgccggaccaagcgcacacggcggccccagcccctcacg

SEQ ID NO: 23 - DNA, 1275 bp, synthetic construct
  misc_feature 1..1275: note = FGF17 fusion
  source 1..1275: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacgggatcgggatcggacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 24 - DNA, 1236 bp, synthetic construct
  misc_feature 1..1236: note = FGF17 deletion mutant
  source 1..1236: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccaccggatcgggatcggacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 25 - DNA, 1167 bp, synthetic construct
  misc_feature 1..1167: note = FGF17 deletion mutant
  source 1..1167: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggatcgggatcggacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 26 - DNA, 1275 bp, synthetic construct
  misc_feature 1..1275: note = FGF17 mutant
  source 1..1275: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccagcggacccagcgcacacggcggccccagcccctcacgggatcgggatcggacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 27 - DNA, 1215 bp, synthetic construct
  misc_feature 1..1215: note = FGF17 deletion mutant
  source 1..1215: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagggatcgggatcggacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 28 - DNA, 1275 bp, synthetic construct
  misc_feature 1..1275: note = FGF17 mutant
  source 1..1275: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgaggctcaggcacagttcgagtttgtgggcgctgcccccacccgccggaccaagcgcacacggcggccccagcccctcacgggatcgggatcggacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 29 - DNA, 1278 bp, synthetic construct
  misc_feature 1..1278: note = FGF17 fusion
  source 1..1278: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacgggatctgggagcgctgacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 30 - DNA, 1239 bp, synthetic construct
  misc_feature 1..1239: note = FGF17 deletion mutant
  source 1..1239: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccaccggatctgggagcgctgacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 31 - DNA, 1170 bp, synthetic construct
  misc_feature 1..1170: note = FGF17 deletion mutant
  source 1..1170: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggatctgggagcgctgacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 32 - DNA, 1278 bp, synthetic construct
  misc_feature 1..1278: note = FGF17 mutant
  source 1..1278: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccagcggacccagcgcacacggcggccccagcccctcacgggatctgggagcgctgacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 33 - DNA, 1218 bp, synthetic construct
  misc_feature 1..1218: note = FGF17 deletion mutant
  source 1..1218: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagggatctgggagcgctgacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 34 - DNA, 1278 bp, synthetic construct
  misc_feature 1..1278: note = FGF17 mutant
  source 1..1278: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgaggctcaggcacagttcgagtttgtgggcgctgcccccacccgccggaccaagcgcacacggcggccccagcccctcacgggatctgggagcgctgacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 35 - DNA, 2406 bp, synthetic construct
  misc_feature 1..2406: note = FGF17 fusion
  source 1..2406: mol_type = other DNA
caccatcaccatcaccatagcggcgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcggaggcggtagcggaggcggtggctccggtggcggagggtctactcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacg

SEQ ID NO: 36 - DNA, 1314 bp, synthetic construct
  misc_feature 1..1314: note = FGF17 fusion
  source 1..1314: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacgggcggaggcggtagcggaggcggtggctccggtggcggagggtctgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 37 - DNA, 1269 bp, synthetic construct
  misc_feature 1..1269: note = FGF17 fusion
  source 1..1269: mol_type = other DNA
actcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacggagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 38 - DNA, 1314 bp, synthetic construct
  misc_feature 1..1314: note = FGF17 fusion
  source 1..1314: mol_type = other DNA
gagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaaggcggaggcggtagcggaggcggtggctccggtggcggagggtctactcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacg

SEQ ID NO: 39 - DNA, 201 bp, Homo sapiens
  source 1..201: mol_type = other DNA
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 40 - DNA, 894 bp, synthetic construct
  misc_feature 1..894: note = IGF2 fusion
  source 1..894: mol_type = other DNA
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggatcgggatcggacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 41 - DNA, 897 bp, synthetic construct
  misc_feature 1..897: note = IGF2 fusion
  source 1..897: mol_type = other DNA
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggatctgggagcgctgacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 42 - DNA, 2025 bp, synthetic construct
  misc_feature 1..2025: note = IGF2 fusion
  source 1..2025: mol_type = other DNA
caccatcaccatcaccatagcggcgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcggaggcggtagcggaggcggtggctccggtggcggagggtctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 43 - DNA, 2025 bp, synthetic construct
  misc_feature 1..2025: note = IGF2 mutant
  source 1..2025: mol_type = other DNA
caccatcaccatcaccatagcggcgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcggaggcggtagcggaggcggtggctccggtggcggagggtctgcttaccgccccagtgagaccctgtgcggcggggagctggtggacaccctccagttcgtctgtggggaccgcggcttctacttcagcaggcccgcaagccgtgtgagcgctcgcagccgtggcatcgttgaggagtgctgtttccgcagctgtgacctggccctcctggagacgtactgtgctacccccgccaagtccgag

SEQ ID NO: 44 - DNA, 2025 bp, synthetic construct
  misc_feature 1..2025: note = IGF2 mutant
  source 1..2025: mol_type = other DNA
caccatcaccatcaccatagcggcgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcggaggcggtagcggaggcggtggctccggtggcggagggtctgcttaccgccccagtgagaccctgtgcggcggggagctggtggacaccctccagttcgtctgtggggaccgcggcttctacttcagcaggcccgcaagccgtgtgagccagcgcagccgtggcatcgttgaggagtgctgtttccgcagctgtgacctggccctcctggagacgtactgtgctacccccgccaagtccgag

SEQ ID NO: 45 - DNA, 2025 bp, synthetic construct
  misc_feature 1..2025: note = IGF2 mutant
  source 1..2025: mol_type = other DNA
caccatcaccatcaccatagcggcgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcggaggcggtagcggaggcggtggctccggtggcggagggtctgcttaccgccccagtgagaccctgtgcggcggggagctggtggacaccctccagttcgtctgtggggaccgcggcttctacttcagcaggcccgcaagccgtgtgagccgtcgcagcgctggcatcgttgaggagtgctgtttccgcagctgtgacctggccctcctggagacgtactgtgctacccccgccaagtccgag

SEQ ID NO: 46 - DNA, 2025 bp, synthetic construct
  misc_feature 1..2025: note = IGF2 mutant
  source 1..2025: mol_type = other DNA
caccatcaccatcaccatagcggcgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcggaggcggtagcggaggcggtggctccggtggcggagggtctgcttaccgccccagtgagaccctgtgcggcggggagctggtggacaccctccagttcgtctgtggggaccgcggcttctacttcagcaggcccgcaagccgtgtgagccgtcgcagccagggcatcgttgaggagtgctgtttccgcagctgtgacctggccctcctggagacgtactgtgctacccccgccaagtccgag

SEQ ID NO: 47 - DNA, 933 bp, synthetic construct
  misc_feature 1..933: note = IGF2 fusion
  source 1..933: mol_type = other DNA
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcggaggcggtagcggaggcggtggctccggtggcggagggtctgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 48 - DNA, 888 bp, synthetic construct
  misc_feature 1..888: note = IGF2 fusion
  source 1..888: mol_type = other DNA
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgaggagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 49 - DNA, 933 bp, synthetic construct
  misc_feature 1..933: note = IGF2 fusion
  source 1..933: mol_type = other DNA
gagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaaggcggaggcggtagcggaggcggtggctccggtggcggagggtctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 50 - DNA, 933 bp, synthetic construct
  misc_feature 1..933: note = IGF2 mutant
  source 1..933: mol_type = other DNA
gcttaccgccccagtgagaccctgtgcggcggggagctggtggacaccctccagttcgtctgtggggaccgcggcttctacttcagcaggcccgcaagccgtgtgagcgctcgcagccgtggcatcgttgaggagtgctgtttccgcagctgtgacctggccctcctggagacgtactgtgctacccccgccaagtccgagggcggaggcggtagcggaggcggtggctccggtggcggagggtctgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 51 - DNA, 417 bp, Homo sapiens
  source 1..417: mol_type = other DNA
tccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccac

SEQ ID NO: 52 - DNA, 1110 bp, synthetic construct
  misc_feature 1..1110: note = BMP7 fusion
  source 1..1110: mol_type = other DNA
tccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccacggatcgggatcggacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 53 - DNA, 1113 bp, synthetic construct
  misc_feature 1..1113: note = BMP7 fusion
  source 1..1113: mol_type = other DNA
tccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccacggatctgggagcgctgacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 54 - protein, 194 aa, Homo sapiens
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 55 - protein, 181 aa, synthetic construct
  REGION 1..181: note = FGF17 deletion mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPT

SEQ ID NO: 56 - protein, 158 aa, synthetic construct
  REGION 1..158: note = FGF17 deletion mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQ

SEQ ID NO: 57 - protein, 194 aa, synthetic construct
  REGION 1..194: note = FGF17 mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTQRTQRTRRPQPLT

SEQ ID NO: 58 - protein, 174 aa, synthetic construct
  REGION 1..174: note = FGF17 deletion mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFE

SEQ ID NO: 59 - protein, 194 aa, synthetic construct
  REGION 1..194: note = FGF17 mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEAQAQFEFVGAAPTRRTKRTRRPQPLT

SEQ ID NO: 60 - protein, 425 aa, synthetic construct
  REGION 1..425: note = FGF17 fusion
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLTGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 412 aa, synthetic construct
  REGION 1..412: note = FGF17 deletion mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 389 aa, synthetic construct
  REGION 1..389: note = FGF17 deletion mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 425 aa, synthetic construct
  REGION 1..425: note = FGF17 mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTQRTQRTRRPQPLTGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 405 aa, synthetic construct
  REGION 1..405: note = FGF17 deletion mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 425 aa, synthetic construct
  REGION 1..425: note = FGF17 mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEAQAQFEFVGAAPTRRTKRTRRPQPLTGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 426 aa, synthetic construct
  REGION 1..426: note = FGF17 fusion
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLTGSGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 413 aa, synthetic construct
  REGION 1..413: note = FGF17 deletion mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTGSGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 390 aa, synthetic construct
  REGION 1..390: note = FGF17 mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGSGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 426 aa, synthetic construct
  REGION 1..426: note = FGF17 mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTQRTQRTRRPQPLTGSGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 406 aa, synthetic construct
  REGION 1..406: note = FGF17 deletion mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEGSGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 426 aa, synthetic construct
  REGION 1..426: note = FGF17 mutant
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEAQAQFEFVGAAPTRRTKRTRRPQPLTGSGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 802 aa, synthetic construct
  REGION 1..802: note = FGF17 fusion
HHHHHHSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 73 - protein, 438 aa, synthetic construct
  REGION 1..438: note = FGF17 fusion
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLTGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 423 aa, synthetic construct
  REGION 1..423: note = FGF17 fusion
TQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 438 aa, synthetic construct
  REGION 1..438: note = FGF17 fusion
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 76 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 77 - protein, 298 aa, synthetic construct
  REGION 1..298: note = IGF2 fusion
AYRPSETLCGGELVDTLQFVCGDRGFYFSRPASRVSRRSRGIVEECCFRSCDLALLETYCATPAKSEGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 299 aa, synthetic construct
  REGION 1..299: note = IGF2 fusion
AYRPSETLCGGELVDTLQFVCGDRGFYFSRPASRVSRRSRGIVEECCFRSCDLALLETYCATPAKSEGSGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 80 - protein, 675 aa, synthetic construct
  REGION 1..675: note = IGF2 fusion
HHHHHHSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSRRSRGIVEECCFRSCDLALLETYCATPAKSE

SEQ ID NO: 81 - protein, 675 aa, synthetic construct
  REGION 1..675: note = IGF2 mutant
HHHHHHSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSARSRGIVEECCFRSCDLALLETYCATPAKSE

SEQ ID NO: 82 - protein, 675 aa, synthetic construct
  REGION 1..675: note = IGF2 mutant
HHHHHHSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSQRSRGIVEECCFRSCDLALLETYCATPAKSE

SEQ ID NO: 83 - protein, 675 aa, synthetic construct
  REGION 1..675: note = IGF2 mutant
HHHHHHSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSRRSAGIVEECCFRSCDLALLETYCATPAKSE

SEQ ID NO: 84 - protein, 675 aa, synthetic construct
  REGION 1..675: note = IGF2 mutant
HHHHHHSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSRRSQGIVEECCFRSCDLALLETYCATPAKSE

SEQ ID NO: 85 - protein, 311 aa, synthetic construct
  REGION 1..311: note = IGF2 fusion
AYRPSETLCGGELVDTLQFVCGDRGFYFSRPASRVSRRSRGIVEECCFRSCDLALLETYCATPAKSEGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 296 aa, synthetic construct
  REGION 1..296: note = IGF2 fusion
AYRPSETLCGGELVDTLQFVCGDRGFYFSRPASRVSRRSRGIVEECCFRSCDLALLETYCATPAKSEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 311 aa, synthetic construct
  REGION 1..311: note = IGF2 fusion
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAYRPSETLCGGELVDTLQFVCGDRGFYFSRPASRVSRRSRGIVEECCFRSCDLALLETYCATPAKSE

SEQ ID NO: 88 - protein, 311 aa, synthetic construct
  REGION 1..311: note = IGF2 mutant
AYRPSETLCGGELVDTLQFVCGDRGFYFSRPASRVSARSRGIVEECCFRSCDLALLETYCATPAKSEGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 90 - protein, 370 aa, synthetic construct
  REGION 1..370: note = BMP7 fusion
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHGSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 371 aa, synthetic construct
  REGION 1..371: note = BMP7 fusion
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHGSGSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - DNA, 96 bp, synthetic construct
  misc_feature 1..96: note = BMP7 knuckle
  source 1..96: mol_type = other DNA
acggtgcccaagccctgctgtgcgcccacgcagctcaatgccatctccgtcctctacttcgatgacagctccaacgtcatcctgaagaaatacaga

SEQ ID NO: 93 - protein, 32 aa, synthetic construct
  REGION 1..32: note = BMP7 knuckle
TVPKPCCAPTQLNAISVLYFDDSSNVILKKYR

SEQ ID NO: 94 - DNA, 12 bp, synthetic construct
  misc_feature 1..12: note = linker
  source 1..12: mol_type = other DNA
ggatcgggatcg

SEQ ID NO: 95 - DNA, 15 bp, synthetic construct
  misc_feature 1..15: note = linker
  source 1..15: mol_type = other DNA
ggatctgggagcgct

SEQ ID NO: 96 - DNA, 45 bp, synthetic construct
  misc_feature 1..45: note = linker
  source 1..45: mol_type = other DNA
ggcggaggcggtagcggaggcggtggctccggtggcggagggtct

SEQ ID NO: 97 - DNA, 681 bp, synthetic construct
  misc_feature 1..681: note = IgG1 Fc mutant
  source 1..681: mol_type = other DNA
gacaaaactcacacatgcccaccgtgcccagcacctgaagctgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtctccgggtaaa

SEQ ID NO: 98 - DNA, 687 bp, synthetic construct
  misc_feature 1..687: note = IgG4 Fc mutant
  source 1..687: mol_type = other DNA
gagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 99 - DNA, 18 bp, synthetic construct
  misc_feature 1..18: note = 6X His
  source 1..18: mol_type = other DNA
caccatcaccatcaccat

SEQ ID NO: 100 - DNA, 24 bp, synthetic construct
  misc_feature 1..24: note = Strept-Tactin binding peptide
  source 1..24: mol_type = other DNA
tggagccacccgcagttcgaaaaa

SEQ ID NO: 101 - DNA, 1755 bp, Homo sapiens
  source 1..1755: mol_type = other DNA
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 102 - protein, 4 aa, synthetic construct
  REGION 1..4: note = linker
GSGS

SEQ ID NO: 103 - protein, 5 aa, synthetic construct
  REGION 1..5: note = linker
GSGSA

SEQ ID NO: 104 - protein, 15 aa, synthetic construct
  REGION 1..15: note = linker
GGGGSGGGGSGGGGS

SEQ ID NO: 105 - protein, 227 aa, synthetic construct
  REGION 1..227: note = IgG1 Fc mutant
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 229 aa, synthetic construct
  REGION 1..229: note = IgG4 Fc mutant
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 6 aa, synthetic construct
  REGION 1..6: note = His tag
HHHHHH

SEQ ID NO: 108 - protein, 8 aa, synthetic construct
  REGION 1..8: note = Step-Tactin binding peptide
WSHPQFEK

SEQ ID NO: 109 - protein, 585 aa, Homo sapiens [sequence omitted]

SEQ ID NO: 110 - protein, 5 aa, synthetic construct
  source 1..5: note = 4GS linker
GGGGS

SEQ ID NO: 111 - protein, 10 aa, synthetic construct
  source 1..10: note = repeat of 2 4GS linkers
GGGGSGGGGS

SEQ ID NO: 112 - protein, 20 aa, synthetic construct
  source 1..20: note = repeat of 4 4GS linkers
GGGGSGGGGSGGGGSGGGGS

SEQ ID NO: 113 - protein, 25 aa, synthetic construct
  source 1..25: note = 5 repeat of 4GS linkers
GGGGSGGGGSGGGGSGGGGSGGGGS

(19) United States
(12) Patent Application Publication
(10) Pub. No.: US 2026/0008828 A1
(43) Pub. Date: Jan. 8, 2026

(54) REGENERATIVE POLYPEPTIDES AND USES THEREOF

(71) Applicant: Juvena Therapeutics, Inc., Redwood City, CA (US)

(72) Inventors: Hanadie YOUSEF, Palo Alto, CA (US); Jeremy O'CONNELL, Palo Alto, CA (US); Thach MAI, Palo Alto, CA (US); Rami JAAFAR, San Francisco, CA (US); Zhihua LI, San Jose, CA (US)

(21) Appl. No.: 19/215,225

(22) Filed: May 21, 2025

Related U.S. Application Data
(63) Continuation of application No. 17/843,748, filed on Jun. 17, 2022, now Pat. No. 12,358,963.
(60) Provisional application No. 62/953,431, filed on Dec. 24, 2019.
(60) Provisional application No. 62/953,429, filed on Dec. 24, 2019.
(60) Provisional application No. 62/953,427, filed on Dec. 24, 2019.

(51) Int. Cl. C07K 14/50; A61K 31/19; A61K 38/30; A61P 19/02; A61P 21/06

(52) U.S. Cl. CPC C07K 14/50; A61K 31/19; A61K 38/30; A61P 19/02; A61P 21/06

(57) ABSTRACT

Described herein are polypeptides comprising an FGF17, IGF2, or BMP7 amino acid sequence and an amino acid sequence from a heterologous polypeptide useful for the treatment of soft-tissue and muscle diseases, disorders, and injuries. Also described herein are synergistic combinations of a Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans. Also described are methods of treating muscle and soft-tissue diseases comprising administering the polypeptides and/or synergistic compositions.

### CROSS-REFERENCE TO RELATED APPLICATIONS

This application is a continuation of U.S. application Ser. No. 17/843,748 filed Jun. 17, 2022, which is a 371 filing from PCT application PCT/US2020/066658 filed on Dec. 22, 2020, which claims priority to U.S. provisional application Ser. No. 62/953,431 filed Dec. 24, 2019, U.S. provisional application Ser. No. 62/953,429 filed Dec. 24, 2019, and U.S. provisional application Ser. No. 62/953,427 filed Dec. 24, 2019, all of which are incorporated by reference in their entirety herein.

### INCORPORATION BY REFERENCE OF SEQUENCE LISTING

The present application is being filed along with a Sequence Listing in electronic format. The Sequence Listing is provided as a file entitled 54275-705_303_SL.xml, created on May 21, 2025, which is 193,913 bytes in size. The information in the electronic format of the Sequence Listing is incorporated by reference in its entirety.

### BACKGROUND

As the average life span increases, increasing emphasis is placed upon “healthy aging.” Individuals would like to live more active lifestyles as they age, and as a result, many aging disorders can have a significant impact on the quality of life of aging individuals. Treatments directed to regenerative ends have utility for treating aging diseases. Additionally, many treatments for aging disorders can be applicable to younger individuals who have suffered illness, injury, or who possess genetic or developmental defects leading to premature tissue loss, wasting, or weakening.

### SUMMARY

Described herein are polypeptides comprising an FGF17, IGF2, or BMP7 amino acid sequence and an amino acid sequence from a heterologous polypeptide useful for the treatment of soft-tissue and muscle diseases, disorders, and injuries. The IGF2 amino acid sequence can include an amino acid sequence at least about 90%, 95%, 97%, 98%, 99%, or 100% identical to the amino acid sequence set forth in SEQ ID NO: 89. The BMP7 amino acid sequence can include an amino acid sequence at least about 90%, 95%, 97%, 98%, 99%, or 100% identical to the amino acid sequence set forth in SEQ ID NO: 89. The BMP7 amino acid sequence can include a 15-30 amino acid fragment at least about 90%, 95%, 98%, 99% or 100% identical to the amino acid sequence set forth in SEQ ID NO: 93. The FGF17 amino acid sequence can include an amino acid sequence at least about 90%, 95%, 97%, 98%, 99%, or 100% identical to the amino acid sequence set forth in SEQ ID NO: 54. The FGF17 amino acid sequence can include a mutation selected from: deletion of amino acids G181-T203, deletion of amino acids 197-T203, deletion of amino acids 204-216, deletion of amino acids 181-216, R204Q/K207Q, deletion of amino acids 197-216, K191A/K193A/S200A, and combinations thereof. The FGF17, IGF2, and/or BMP7 can include at least one amino acid that is N-, C-, or O-linked glycosylated.

The heterologous polypeptide can be an immunoglobulin molecule or fragment thereof, an albumin molecule, a transferrin molecule, an XTEN sequence, a proline-alanine-serine polymer, a homo-amino acid polymer, a glycine-rich sequence, a gelatin-like polymer, an elastin-like peptide, a carboxy-terminal peptide, or combinations thereof. A fragment of an immunoglobulin molecule can include the hinge domain of an IgG, the CH2 domain of an IgG, the CH3 domain of an IgG, or any combination thereof. The immunoglobulin molecule or fragment thereof can include one or more mutations that reduce the effector function of the fragment of the immunoglobulin molecule. Also described herein are combinations of a Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans.

Described herein are methods of treating muscle and soft-tissue diseases comprising administering the polypeptides and/or compositions combining a polypeptide with a small chain fatty acid, mTOR activator, and/or glycosaminoglycan. Muscle diseases that can be treated include, for example, acute and chronic muscle wasting diseases or conditions, such as sarcopenia, cachexia, muscular dystrophies, and muscle injury. Soft tissue regeneration can be useful to treat acute and chronic muscle wasting diseases or conditions.

### BRIEF DESCRIPTION OF THE DRAWINGS

FIG. 1A depicts purified IGF2-hFcm promoted differentiation of human myoblast cells.

FIG. 2A depicts sodium butyrate enhanced muscle fusion.

FIG. 2B depicts sodium butyrate enhanced IGF2 activity.

FIG. 2C depicts sodium butyrate enhanced IGF2 activity.

FIG. 3A depicts the change in percent area of eMyHC positive cells treated with additional doses of vehicle, IGF2, sodium butyrate, or IGF2 and sodium butyrate.

FIG. 3B depict the change in percent area of eMyHC positive cells treated with additional doses of vehicle, IGF2, sodium butyrate, or IGF2 and sodium butyrate.

FIG. 4A depicts IGF2 Receptor was expressed on chondrocyte and osteocytes.

FIG. 5A depicts BMP7 induced myoblast proliferation as measured by newly formed nuclei.

FIG. 5B depicts BMP7 induced myoblast proliferation as measured by total nuclei.

FIG. 6A depicts leucine enhanced BMP7 mitogenic activity.

FIG. 7A depicts hyaluronic acid (HA) enhanced BMP7 mitogenic activity.

FIG. 8A depicts BMP7 receptors were expressed in human myoblast.

FIG. 9A depicts treatment for chondrocyte proliferation in cartilage injury and osteoarthritis.

FIG. 10A depicts FGF17-hFcm promoted proliferation of mouse myoblasts as a part of cultured media supernatant, or purified (FIG. 10B).

FIG. 11A depicts FGF17 sequence mutations improved protein expression levels in CHO cells.

FIG. 11B depicts FGF17 sequence mutations promoted proliferation of mouse myoblasts as a part of cultured media supernatant, or purified.

FIG. 12A depicts additional FGF17 mutants improved protein expression levels in CHO cells.

FIG. 12B depicts additional FGF17 sequence mutations promoted proliferation of mouse myoblasts as a part of cultured media supernatant, or purified.

FIG. 13A depicts FGF17 receptor was expressed in human myoblasts.

FIG. 14A depicts heparin enhanced FGF17 mitogenic activity.

FIG. 15A depicts hyaluronic acid (HA) enhanced FGF17 mitogenic activity.

FIG. 16A depicts an experimental overview that demonstrated intramuscular administration of FGF17 promoted the regeneration of muscle in BaCl2 injured old mice model.

FIG. 16B depicts intramuscular administration of FGF17 promoted the regeneration of muscle in BaCl2 injured old mice model as measured by new fiber formation.

FIG. 16C depicts intramuscular administration of FGF17 promoted the regeneration of muscle in BaCl2 injured old mice model by reducing fibrosis.

FIG. 17A depicts an experimental overview that demonstrated systemic administration of FGF17 protects against Dexamethasone induced muscle atrophy.

FIG. 17B-D depicts systemic administration of FGF17 protected against Dexamethasone induced muscle atrophy as measured by percent muscle mass change (FIG. 17B), forelimb specific force (FIG. 17C) and bothlimb force (FIG. 17D).

### DETAILED DESCRIPTION

In certain aspects disclosed herein is a therapeutically active protein or polypeptide sequence or derivative or fragment thereof that enhances progenitor cell growth or regeneration or function through activation of a cell surface receptor, and one or more of: a secretion signal a multimerizing component, or a stabilizing component. We modify and combined the sequences of certain polypeptides to create secreted, therapeutically active proteins with applications to muscle and soft tissue regeneration useful to treat acute and chronic muscle wasting diseases or conditions, such as sarcopenia, cachexia, muscular dystrophies, and muscle injury. In certain aspects, disclosed herein is a method of treating individuals with acute and chronic muscle wasting diseases or conditions, such as sarcopenia, cachexia, muscular dystrophies, and muscle injury.

In certain aspects, disclosed herein is a polypeptide comprising an FGF8 subfamily amino acid sequence and a heterologous polypeptide amino acid sequence, wherein the heterologous polypeptide increases the stability or biological function of the FGF8 subfamily amino acid sequence. In certain aspects, disclosed herein is a composition comprising an FGFR agonist and a glycosaminoglycan.

In certain aspects, disclosed herein is a polypeptide comprising an IGF2 amino acid sequence and a heterologous polypeptide amino acid sequence, wherein the heterologous polypeptide amino acid sequence increases the stability or biological function of the IGF2 amino acid sequence. In certain aspects, disclosed herein is a composition comprising an IGF1R agonist and a short fatty acid chain.

In certain aspects, disclosed herein is a polypeptide comprising a BMP7 amino acid sequence and a heterologous polypeptide amino acid sequence, wherein the heterologous polypeptide increases the stability or biological function of the BMP7 amino acid sequence. In certain aspects, disclosed herein is a composition comprising a BMP7 receptor agonist and a glycosaminoglycan.

The secretion signal sequence can either be one naturally occurring with a therapeutically active protein or polypeptide sequence or a different one selected, modified, or created to optimize expression yield through secretion efficiency, processing kinetics, or cell line specific processing. Further examples and SEQ IDs are in Table 1. In certain aspects, the polypeptide may comprise a secretory signal peptide. In certain embodiments, the secretory signal peptide is one of SEQ ID NO: 10-16. Production of the fusion polypeptides may be done in heterologous production systems (e.g., bacteria, yeast, mammalian, inset, etc.).

Polypeptides can induce a regenerative effect through membrane receptors in desired cell types. Examples from the stem cell secretome selected for their ability to improve muscle and soft tissue regeneration are listed in Table 2, and include, for example, FGF17, BMP7, IGF2, and variants thereof. Multimerizing components can join two or more other protein components together. A multimerizing component can take the form of a linker sequence of amino acids that joins other components tandemly into a single consecutive amino acid sequence. Or multimerizing components can take the form of proteins or protein domains that dimerize, resulting in covalent disulfide linking or non-covalently associations driving dimerization. Examples are found in Table 3.

Stabilizing components can reduce degradation, increase translational or post-translation folding, reduce unfolding rates, increase half-life, and/or improve other desirable pharmacokinetic parameters (e.g., serum half-life, Cmax, AUC, Tmax, etc.). Examples can include abundant, circulating proteins or fragments thereof such as albumin or the fragment crystallizable (Fc) region from a human antibody. Further examples are in Table 3.

### Certain Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10%.

As used herein the terms “individual,” “patient,” or “subject” are used interchangeably and refer to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

As used herein the term “treat” or “treating” refers to interventions to a physiological or disease state of an individual designed or intended to ameliorate at least one sign or symptom associated with said physiological or disease state. The skilled artisan will recognize that given a heterogeneous population of individuals afflicted with a disease, not all individuals will respond equally, or at all, to a given treatment.

As used herein, the term “heterologous” refers to a nucleotide or amino acid sequence that is from a different source (e.g., gene, polypeptide, or organism) compared to the amino acid or nucleotide sequence to which it refers to as being heterologous. Heterologous includes biological sequences derived from different organisms or to sequences derived from different sources (e.g., genes or proteins) of the same organism. Heterologous sequences include recombinant DNA molecules comprising nucleotide sequences from different sources, fusion proteins comprising amino acid sequences from different sources, and epitope or purification tags of natural or synthetic origin.

As used herein, the term “muscle” refers to skeletal muscle, and does not refer to smooth muscle or cardiac muscle.

As used herein, the term “soft tissue” refers to connective tissues, including without limitations, tendons, ligaments, and cartilage.

As used herein, the term “mitogenic activity” refers to an activity that induces cell division or proliferation.

As used herein, the term “fusion promoting activity” refers to activity that promotes the fusion of cells into multinucleated cells, such as the fusion of myocytes into multinucleated myofibers, or advances the differentiation of a terminal differentiating stem or progenitor cells toward a committed cell lineage type, such as the progression of myoblasts into myocytes or the increase in cell size of expanding myofibers.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

### FGF17 Polypeptides

In certain aspects, described herein, are FGF17 polypeptides that comprise an FGF17 amino acid sequence. The FGF17 amino acid sequence can be a human FGF17. The FGF17 amino acid sequence can have at least about 90%, 95%, 97%, 98%, 99%, or 100% sequence identity to SEQ ID NO: 54. The FGF17 amino acid sequence can be 100% identical to SEQ ID NO: 54. The FGF17 amino acid sequence can be at least about 90%, 95%, 97%, 98%, 99%, or 100% identical to SEQ ID NO: 55. The FGF17 amino acid sequence can be 100% identical to SEQ ID NO: 55. The FGF17 amino acid sequence can be at least about 90%, 95%, 97%, 98%, 99%, or 100% identical to SEQ ID NO: 56. The FGF17 amino acid sequence can be 100% identical to SEQ ID NO: 56. The FGF17 amino acid sequence can be at least about 90%, 95%, 97%, 98%, 99%, or 100% identical to SEQ ID NO: 57, wherein the sequence comprises the R204Q and K207Q mutations. The FGF17 amino acid sequence can be 100% identical to SEQ ID NO: 57. The FGF17 amino acid sequence can be at least about 90%, 95%, 97%, 98%, 99%, or 100% identical to SEQ ID NO: 58. The FGF17 amino acid sequence can be 100% identical to SEQ ID NO: 58. The FGF17 amino acid sequence can be at least about 90%, 95%, 97%, 98%, 99%, or 100% identical to SEQ ID NO: 59, wherein the sequence comprises the K191A, K193A, and S200A mutations. The FGF17 amino acid sequence can be 100% identical to SEQ ID NO: 59.

The FGF17 polypeptides described herein can be fusion proteins or polypeptides that may comprise additional heterologous (non-FGF17) amino acid sequences that enhance the expression, stability or function of the FGF17 polypeptide. These heterologous amino acid sequences may increase the expression of the FGF17 fusion polypeptide from a cell system (e.g., CHO cells or other suitable cell system for bulk production) by 10%, 20%, 25%, 30%, 40%, 50%, 75%, 100%, 150%, 200%, 200%, 400%, 500%, 1,000% or more compared to a polypeptide not comprising the heterologous amino acid sequence. These heterologous amino acid sequences may increase the bioavailability (e.g., increasing the T½) of the FGF17 polypeptide in vivo by 10%, 20%, 25%, 30%, 40%, 50%, 75%, 100%, 150%, 200%, 200%, 400%, 500%, 1,000% or more compared to a polypeptide not comprising the heterologous amino acid sequence. These heterologous amino acid sequences may increase the function (e.g., signaling through an FGF receptor) of the FGF17 polypeptide in vivo by 10%, 20%, 25%, 30%, 40%, 50%, 75%, 100%, 150%, 200%, 200%, 400%, 500%, 1,000% or more compared to a polypeptide not comprising the heterologous amino acid sequence.

The FGF17 amino acid sequence of the FGF17-heterologous polypeptide fusion protein can be at least about 90%, 95%, 97%, 98%, 99%, or 100% identical to SEQ ID NO: 54. The FGF17 amino acid sequence of the fusion protein can be 100% identical to SEQ ID NO: 54. The FGF17 amino acid sequence of the fusion protein can be at least about 90%, 95%, 97%, 98%, 99%, or 100% identical to SEQ ID NO: 55. The FGF17 amino acid sequence of the fusion protein can be 100% identical to SEQ ID NO: 55. The FGF17 amino acid sequence of the fusion protein can be at least about 90%, 95%, 97%, 98%, 99%, or 100% identical to SEQ ID NO: 56. The FGF17 amino acid sequence of the fusion protein can be 100% identical to SEQ ID NO: 56. The FGF17 amino acid sequence of the fusion protein can be at least about 90%, 95%, 97%, 98%, 99%, or 100% identical to SEQ ID NO: 57, wherein the sequence comprises the R204Q and K207Q mutations. The FGF17 amino acid sequence of the fusion protein can be 100% identical to SEQ ID NO: 57. The FGF17 amino acid sequence of the fusion protein can be at least about 90%, 95%, 97%, 98%, 99%, or 100% identical to SEQ ID NO: 58. The FGF17 amino acid sequence of the fusion protein can be 100% identical to SEQ ID NO: 58. The FGF17 amino acid sequence of the fusion protein can be at least about 90%, 95%, 97%, 98%, 99%, or 100% identical to SEQ ID NO: 59, wherein the sequence comprises the K191A, K193A, and S200A mutations. The FGF17 amino acid sequence of the fusion protein can be 100% identical to SEQ ID NO: 59.

An FGF17 fusion polypeptide amino acid sequence can be at least about 90%, 95%, 97%, 98%, 99%, or 100% identical to SEQ ID NO: 60, SEQ ID NO: 61, or SEQ ID NO: 62. The fusion polypeptide amino acid sequence can be at least about 90%, 95%, 97%, 98%, 99%, or 100% identical to SEQ ID NO: 65, wherein the sequence comprises the R204Q and K207Q mutations. The fusion polypeptide amino acid sequence can be 100% identical to SEQ ID NO: 66, SEQ ID NO: 66 or SEQ ID NO: 71, wherein the sequence comprises the K191A, K193A, and S200A mutations. The fusion polypeptide amino acid sequence can be 100% identical to SEQ ID NO: 72. The fusion polypeptide amino acid sequence can be at least about 90%, 95%, 97%, 98%, 99%, or 100% identical to SEQ ID NO: 73, SEQ ID NO: 74, SEQ ID NO: 75, or SEQ ID NO: 69, wherein the sequence comprises the R204Q and K207Q mutations.

The FGF17 amino acid sequence can be at least about 80%, 90%, 95%, 97%, 98%, 99% or 100% identical to one of SEQ ID NO: 54-70 or 74, with 1, 2, 3, 4, 5, 6, 7, 8, 9, or 10 amino acids are deleted from the N- and/or C-terminus of the polypeptide.

### IGF2 Fusion Proteins

Described herein are certain therapeutically useful IGF2 polypeptides, including IGF2 fusion polypeptides that promote in vivo stability and function of the IGF2 comprising polypeptides.

In certain aspects described herein are IGF receptor ligand polypeptides. The IGF2 polypeptides can comprise an IGF2 amino acid sequence. The IGF2 amino acid sequence can be that of a human IGF2 polypeptide. The human IGF2 polypeptide can comprise amino acids 25 to 91 of SEQ ID NO. 79 (i.e. SEQ ID NO. 76). The IGF2 amino acid sequence can be at least about 90%, 95%, 97%, 98%, 99%, or 100% identical to one of SEQ ID NO. 76, 79, 81 or 86. The IGF2 amino acid sequence can be 100% identical to SEQ ID NO. 76. The IGF2 amino acid sequence can be at least about 60%, 70%, 80%, 90%, 95%, 97%, 98%, 99% or 100% identical to one of SEQ ID NO: 76, 79-81, 86, or 88 and 1, 2, 3, 4, 5, 6, 7, 8, 9, or 10 amino acids are deleted from the N- and/or C-terminus of the polypeptide.

In certain IGF2 polypeptides described herein are fusion proteins or polypeptides that may comprise additional heterologous (non-IGF2) amino acid sequences that enhance the expression, stability or function of the IGF2 polypeptide compared to a polypeptide not comprising the heterologous amino acid sequence. These heterologous amino acid sequences may increase the expression of the IGF2 fusion polypeptide from a cell system (e.g., CHO cells or other suitable cell system for bulk production) by 10%, 20%, 25%, 30%, 40%, 50%, 75%, 100%, 150%, 200%, 200%, 400%, 500%, 1,000% or more compared to a polypeptide not comprising the heterologous amino acid sequence. These heterologous amino acid sequences may increase the bioavailability or other pharmacokinetic factor (e.g., increasing the T½, AUC, Cmax, Tmax, etc.) of the IGF2 polypeptide in vivo by 10%, 20%, 25%, 30%, 40%, 50%, 75%, 100%, 150%, 200%, 200%, 400%, 500%, 1,000% or more compared to a polypeptide not comprising the heterologous amino acid sequence. These heterologous amino acid sequences may improve the pharmacodynamics and/or increase the function (e.g., signaling through an IGF receptor) of the IGF2 polypeptide in vivo by 10%, 20%, 25%, 30%, 40%, 50%, 75%, 100%, 150%, 200%, 200%, 400%, 500%, 1,000% or more compared to a polypeptide not comprising the heterologous amino acid sequence.

Also described herein are IGF receptor ligand fusion polypeptides or polypeptides that include an amino acid sequence heterologous to IGF2. The IGF receptor ligand fusion includes a heterologous amino acid sequence that promotes the stability or function of the IGF receptor ligand. The IGF2 amino acid sequence of the IGF2-heterologous polypeptide fusion protein can be at least about 90%, 95%, 97%, 98%, 99%, or 100% identical to SEQ ID NO. 76. The IGF2 amino acid sequence of the fusion protein can be 100% identical to SEQ ID NO. 76. The IGF2 amino acid sequence of the fusion protein can be at least about 90%, 95%, 97%, 98%, 99%, or 100% identical to SEQ ID NO. 80. The IGF2 amino acid sequence of the fusion protein can be 100% identical to SEQ ID NO. 80. The IGF2 amino acid sequence of the fusion protein can be at least about 90%, 95%, 97%, 98%, 99%, or 100% identical to SEQ ID NO. 88. The IGF2 amino acid sequence of the fusion protein can be 100% identical to SEQ ID NO. 88. Additional representative sequences can be found in Table 2.

### BMP7 Fusion Proteins

Described herein are certain therapeutically useful BMP7 polypeptides, including BMP7 fusion polypeptides that promote in vivo stability and function of the BMP7 comprising polypeptides.

In one aspect, described herein, are BMP7 polypeptides, that comprise a BMP7 amino acid sequence. The BMP7 amino acid sequence can be a human BMP7 amino acid sequence. The BMP7 amino acid sequence can comprise or consist of amino acids 293 to 431 of BMP7. The BMP7 amino acid sequence can comprise a BMP7 knuckle domain (SEQ ID NO: 92). The BMP7 sequence can be at least about 90%, 95%, 97%, 98%, 99%, or 100% identical to SEQ ID NO: 89. The BMP7 sequence can be 100% identical to SEQ ID NO: 89. The BMP7 sequence can be at least about 90%, 95%, 97%, 98%, 99%, or 100% identical to SEQ ID NO: 92. The BMP7 sequence can be 100% identical to SEQ ID NO: 92. The BMP7 polypeptide sequence can comprise one, two, three, four or more repeats of a BMP7 knuckle domain.

The BMP7 amino acid sequence may be further fused to a heterologous amino acid sequence, either directly or with a linker sequence between the BMP7 amino acid sequence and the heterologous polypeptide amino acid sequence to create a fusion polypeptide. The fusion polypeptide can comprise a human BMP7 amino acid sequence. The fusion polypeptide can comprise or consist of amino acids 293 to 431 of BMP7. The fusion polypeptide can comprise a BMP7 knuckle domain (SEQ ID NO: 92). The fusion polypeptide amino acid sequence can comprise a BMP7 amino acid sequence at least about 90%, 95%, 97%, 98%, 99%, or 100% identical to SEQ ID NO: 89. The fusion polypeptide can comprise a BMP7 amino acid sequence 100% identical to SEQ ID NO: 89. The fusion polypeptide amino acid can comprise a BMP7 amino acid sequence at least about 90%, 95%, 97%, 98%, 99%, or 100% identical to SEQ ID NO: 92. The fusion polypeptide can comprise a BMP7 amino acid sequence 100% identical to SEQ ID NO: 92. The fusion polypeptide amino acid sequence can be at least about 90%, 95%, 97%, 98%, 99%, or 100% identical to SEQ ID NO: 90. The fusion polypeptide amino acid sequence can be 100% identical to SEQ ID NO: 90. The fusion polypeptide amino acid sequence can be at least about 90%, 95%, 97%, 98%, 99%, or 100% identical to SEQ ID NO: 91. The fusion polypeptide amino acid sequence can be 100% identical to SEQ ID NO: 91.

The BMP7 amino acid sequence can be at least about 80%, 90%, 95%, 97%, 98%, 99% or 100% identical to one of SEQ ID NO: 20, 21, 32, or 89, with 1, 2, 3, 4, 5, 6, 7, 8, 9, or 10 amino acids deleted from the N- and/or C-terminus of the polypeptide.

In some aspects, the BMP7 sequence may be a fragment of a BMP7 sequence. The knuckle domain of BMP7 comprises amino acids 98 to 129 of SEQ ID NO: 89 (SEQ ID NO: 32). 15-30 amino acid fragments from the knuckle domain can activate BMP signaling. The BMP7 sequence can comprise a knuckle domain of BMP. The BMP7 sequence can be a 15-30 amino acid fragment of SEQ ID NO: 32. The BMP7 sequence can be at least about 13, 14, 15, 16, 17, 18, 19, 20, 21, 22, 23, 24, 25, 26, 27, 29, 29, 30, 31, 32, 33, 34, or 35 amino acids of SEQ ID NO: 32.

### Secretory Signal Peptides

In certain aspects, the fusion polypeptide may comprise a secretory signal peptide. The secretory signal peptide can be SEQ ID NO. 24, SEQ ID NO 25, SEQ ID NO 26, SEQ ID NO 27, SEQ ID NO 28, SEQ ID NO 29, or SEQ ID NO: 30. Production of the fusion polypeptides herein in heterologous production systems (e.g., bacteria, yeast, mammalian, insect, etc.) may involve the use of an appropriate secretory signal sequence for the particular host cell.

Multimerizing components join two or more other protein components. A multimerizing component may comprise a linker sequence of amino acids that joins other components that are identical or different into a single consecutive amino acid sequence. Suitable linkers include polypeptide linkers such as a Gly-Ser linker or spacer described herein. A multimerizing component can take the form of proteins or protein domains that multimerize or dimerize, resulting in covalent disulfide linking (e.g., through the addition of one or more de novo cysteine residues) or non-covalent associations driving dimerization (e.g., a leucine zipper). The multimerizing components may link or multimerize a plurality of IGF2 amino acid sequences. The multimerizing components may link or multimerize two IGF2 amino acid sequences. The two IGF2 amino acid sequences may be the same, or different, and selected from any of the IGF2 sequences described herein. The multimerizing components may link or multimerize two, three, four, five or more IGF2 amino acid sequences. The multimerizing components may link or multimerize an IGF2 amino acid sequence with another polypeptide that provides fusion promoting, proliferation promoting function, increased plasma half-life, or improvement of other pharmacokinetic or pharmacodynamic parameters.

The FGF17, IGF2, or BMP7 amino acid sequence may comprise functional fragments, mutated sequences, or modified polypeptides thereof. Table 2 lists some exemplary fragments, polypeptides and modified polypeptides. The IGF2 or BMP7 sequence can be N-, C-, or O-linked glycosylated. The IGF2 sequence can be glycosylated at one amino acid. The IGF2 sequence can be glycosylated at a site corresponding to Thr96, Thr99, or Thr163 of SEQ ID NO. 31. The BMP7 sequence can comprise at least one glycosylated amino acid. The BMP7 can be glycosylated at residues Asn10, Asn29, or Asn90 of SEQ ID NO. 89.

The FGF17, IGF2, or BMP7 receptor ligand polypeptides and receptor ligand fusion polypeptides described herein may be encoded by nucleic acids to facilitate production of the receptor ligand polypeptide or fusion polypeptide. These nucleic acids can be compatible with bacterial, yeast, insect, or mammalian expression systems. They may comprise promoters/enhancers (either constructive or inducible), polyadenylation signals, selectable markers (such as antibiotic resistance), origins of replication or other accessory nucleic acid sequences. FGF17, IGF2, or BMP7 sequences can be used from many organisms. The FGF17, IGF2, or BMP7 sequence can comprise a human FGF17, IGF2, or BMP7 amino acid sequence. The FGF17, IGF2, or BMP7 sequence can comprise a cat, dog or a horse FGF17, IGF2, or BMP7 sequence. The FGF17, IGF2, or BMP7 sequence can comprise a mouse, rat, rabbit, dog, cat, horse, cow, sheep, pig, goat, llama, alpaca, yak, or monkey sequence.

### FGF17 Nucleic Acid Sequences

In certain embodiments, the FGF17 nucleic acid sequence is at least about 90%, 95%, 97%, 98%, 99%, or 100% identical to SEQ ID NO. 17. The FGF17 nucleic acid sequence can be 100% identical to SEQ ID NO. 17. The FGF17 nucleic acid sequence is at least about 90%, 95%, 97%, 98%, 99%, or 100% identical to SEQ ID NO. 22. The FGF17 nucleic acid sequence can be 100% identical to SEQ ID NO. 22. The FGF17 nucleic acid sequence can be at least about 90%, 95%, 97%, 98%, 99%, or 100% identical to SEQ ID NO. 23. The FGF17 nucleic acid sequence can be 100% identical to SEQ ID NO. 23.

### IGF2 Nucleic Acid Sequences

The IGF2 nucleic acid sequence can be at least about 90%, 95%, 97%, 98%, 99%, or 100% identical to SEQ ID NO. 39. The IGF2 nucleic acid sequence can be 100% identical to SEQ ID NO. 39. The IGF2 nucleic acid sequence can be at least about 90%, 95%, 97%, 98%, 99%, or 100% identical to SEQ ID NO. 43. The IGF2 nucleic acid sequence can be 100% identical to SEQ ID NO. 43. The IGF2 nucleic acid sequence can be at least about 90%, 95%, 97%, 98%, 99%, or 100% identical to SEQ ID NO. 46. The IGF2 nucleic acid sequence can be 100% identical to SEQ ID NO. 46.

### BMP7 Nucleic Acid Sequences

The BMP7 nucleic acid sequence can be at least about 90%, 95%, 97%, 98%, 99%, or 100% identical to SEQ ID NO: 51. The BMP7 nucleic acid sequence can be 100% identical to SEQ ID NO: 51. The BMP7 nucleic acid sequence can be at least about 90%, 95%, 97%, 98%, 99%, or 100% identical to SEQ ID NO: 52. The BMP7 nucleic acid sequence can be 100% identical to SEQ ID NO: 52. The BMP7 nucleic acid sequence can be at least about 90%, 95%, 97%, 98%, 99%, or 100% identical to SEQ ID NO: 53. The BMP7 nucleic acid sequence can be 100% identical to SEQ ID NO: 53.

### Heterologous Peptides

The heterologous polypeptide that comprises part of the fusion proteins described herein may comprise, consist, or consist essentially of a fragment of an immunoglobulin molecule, an albumin molecule, a transferrin molecule, an XTEN sequence, a proline-alanine-serine polymer, a homo-amino acid polymer, a glycine-rich sequence, a gelatin-like polymer, an elastin-like peptide, a carboxy-terminal peptide, or combinations thereof.

In one aspect described herein the therapeutic polypeptide is FGF receptor ligand polypeptide or an FGF17 polypeptide. In one aspect described herein the therapeutic polypeptide is IGF receptor ligand polypeptide or an IGF2 polypeptide. In one aspect described herein the therapeutic polypeptide is BMP receptor ligand polypeptide or an BMP7 polypeptide.

In one aspect described herein the therapeutic polypeptide fused to a heterologous polypeptide amino acid sequence, either directly or through a linker, wherein the heterologous amino acid sequence imparts increased function or stability to the therapeutic polypeptide.

The heterologous peptide can improve the pharmacokinetics, pharmacodynamics, stability or biological function of the therapeutic amino acid sequence. The heterologous sequence may be fused to the therapeutic amino acid sequence at the C-terminus or at the N-terminus of the therapeutic amino acid sequence. The therapeutic amino acid sequence can be fused to a heterologous sequence at the N-terminus. The therapeutic amino acid sequence can be fused to a heterologous sequence at the C-terminus. A flexible linker can be used between the therapeutic amino acid sequence and the heterologous sequence at the N terminus. A flexible linker can be used between the therapeutic amino acid sequence and the heterologous sequence at the C terminus. A spacer can be used between the therapeutic amino acid sequence and the heterologous sequence at the N terminus. A spacer can be used between the therapeutic amino acid sequence and the heterologous sequence at the C terminus.

Heterologous peptides can improve the pharmacokinetics, pharmacodynamics, stability, or the biological function of the IGF2 amino acid sequence. Fusion proteins can be used to improve the pharmacokinetics of the biologically active molecules, such as by prolonging the half-life, as discussed in Strohl, “Fusion Proteins for Half-Life Extension of Biologics as a Strategy to Make Biobetters,” BioDrugs (2015) 29:215-239. Fusing a polypeptide to a molecule or a fragment of a molecule with a long half-life, such as an immunoglobulin, an albumin, or a transferrin increase the half-life of the polypeptide. An XTEN sequence is a repeating amino acid polymer containing the amino acid residues A, E, G, P, S, and T which when fused to a peptide is capable of extending the half-lives of the peptides, while being otherwise inert. Fusing small repeating sequences such as proline-alanine-serine polymers (repeats of proline, alanine and serine), a homo-amino acid polymer sequence such as glycine-rich sequences (G-G-G-S), gelatin-like proteins, and elastin-like sequences (V-P-G-x-G, where x is any amino acid except proline) can also extend the half-life of a polypeptide. Fusing a polypeptide to a carboxy-terminal peptide (CTP) can increase the half-life of the polypeptide in the serum due to the strong negative change of CTP. The heterologous polypeptide can comprise a fragment of an immunoglobulin molecule, an albumin molecule, a transferrin molecule, an XTEN sequence, a proline-alanine-serine polymer, a homo-amino acid polymer, a glycine-rich sequence, a gelatin-like polymer, an elastin-like peptide, a carboxy-terminal peptide, or combinations thereof.

Immunoglobulins are large effector molecules and, for example, IgG immunoglobulins have a plasma half-life of approximately 21 days. When an immunoglobulin fragment is fused to second polypeptide, this can increase the half-life of the second polypeptide. The fragment of the immunoglobulin molecule can comprise the hinge domain of an IgG, the CH2 domain of an IgG, the CH3 domain of an IgG, or any combination thereof. The fragment of the immunoglobulin molecule can comprise the hinge domain of IgG1, the CH2 domain of IgG1, the CH3 domain of IgG1, or any combination thereof. The fragment of the immunoglobulin molecule can comprise the hinge domain of IgG4, the CH2 domain of IgG4, the CH3 domain of IgG4, or any combination thereof.

In some circumstances, mutations of the immunoglobulin molecule or fragment may increase the half-life or stability of the immunoglobulin molecule or fragment. The fragment of the immunoglobulin molecule can comprise the hinge domain of IgG1, the CH2 domain of IgG1, the CH3 domain of IgG1, or any combination thereof with one or more of the following amino acid mutations in the immunoglobulin molecule: P329G, L234A and L235A. The fragment of the immunoglobulin molecule comprises an IgG4 molecule. The fragment of the immunoglobulin molecule can comprise an IgG4 molecule with at least one of the following amino acid mutations in the immunoglobulin molecule: N434A, N434H, T307A/E380A/N434A, M252Y/S254T/T256E, 433K/434F/436H, T250Q, T250F, M428L, M428F, T250Q/M428L, N434S, V308W, V308Y, V308F, M252Y/M428L, D2591/V308F, M428L/V308F, Q311V/N434S, T307Q/N434A, E258F/V427T, S228P, L235E, S228P/L235E/R409K, S228P/L235E, K370Q, K370E, deletion of G446, deletion of K447, and combinations thereof of IgG4 according to the EU numbering system.

Secretory signal sequences are sequence motifs that target proteins to the secretory pathway in the cell. Secretory sequences may be cleaved from the protein to produce the mature, secreted protein. The polypeptide can comprise a secretory signal sequence. The polypeptide can comprise human FGF17, IGF2, or BMP7 secretory sequence (SEQ ID NO 9, SEQ ID NO 11, SEQ ID NO 12). The polypeptide can comprise a secretory signal that is SEQ ID NO. 10, SEQ ID. NO. 13, SEQ ID NO. 14, SEQ ID NO. 15, or SEQ ID NO. 16.

### Linkers and Spacers

Linkers or spacers are short amino acid sequences that separate different domains in a single protein, or domains between fusion proteins. As used herein, the term “linker” and spacer” are interchangeable. Linkers can either be rigid or flexible. Rigid linkers may prevent unwanted interactions between different domains. Proline-rich linkers tend to be more rigid, while glycine rich linkers tend to be more flexible. Flexible linkers may allow domains within a single protein to interact. Another use for flexible linkers is to covalently bond protein complexes and binding partners to generate stable protein complexes. Flexible linkers may also be used to promote dimerization. Linkers and spacers are reviewed in Chichili et al, Linkers in the Structural biology of protein-protein interactions, Protein Sci. February 2013. 22(2): 153-167.

The fusion polypeptides described herein may further comprise a linker or a spacer amino acid sequence that separate the therapeutics polypeptide and the heterologous polypeptide. The linker or spacer can be a peptide linker or spacer. The linker or spacer can be a flexible linker or spacer. The linker can be three alanines (AAA). The peptide linker can be a glycine-serine linker. The linker can be (in one-letter amino acid code): GGGGS (4GS) or multimers of the 4GS linker, such as repeats of 2, 3, 4, or 5 4GS linkers. The glycine-serine linker can comprise the amino acid sequence set forth in SEQ ID NO: 94 or 95, or 2, 3, 4, 5, or repeats of SEQ ID NO: 94 or 95. The linker can comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 amino acids derived from neither the polypeptide sequences in Table 2 nor the heterologous polypeptide amino acid sequences of Table 3.

The linker or spacers can be a single amino acid residue or greater in length. In certain embodiments, the peptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 amino acids in length. The peptide linker can have at least one amino acid residue but is no more than 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acid residues in length.

### Combinations of FGFR Agonists and Glycosaminoglycans

In certain aspects, disclosed herein is a composition comprising an FGFR agonist and a glycosaminoglycan. There are four FGF receptors, FGF1R, FGFR2, FGFR3, FGFR4, which are expressed by a variety of tissues throughout the body. Chemical agonists of FGFR include without limitations, PF-05231023 and SUN11602. Other polypeptides, including dekafin and hexafins, are also capable of activating FGFR signaling. These compositions can comprise an unexpected synergistic effect and are useful for treating the muscle and/or soft-tissue conditions or disorders. This synergistic effect may also be promoted by methods comprising separate administration of an FGFR agonist and a glycosaminoglycan. The combinations described herein can impart additional treatment utility and function to each of the individual components of the combination.

FGFR1 can be activated by FGF1, FGF2, FGF3, FGF4, FGF5, FGF6, FGF8, FGF10, FGF17, FGF19, FGF20, FGF21, FGF22, and FGF23. The FGFR1 agonist can be an FGFR1 agonistic antibody, an FGF polypeptide or a functional fragment thereof, FGF17 or a functional fragment thereof, PF-05231023, SUN11602, a dekafin, a hexafin, or combinations thereof.

FGFR2 comprises two alternatively spliced isoforms. FGFR2IIIb binds to FGF1, FGF3, FGF10 and FGF22, while FGFR2IIIc binds to FGF1, FGF2, FGF4, FGF6, FGF8, FGF9, FGF17, and FGF18. The FGFR2 agonist can be an FGFR2 agonistic antibody, an FGF polypeptide or a functional fragment thereof, FGF17 or a functional fragment thereof, PF-05231023, SUN11602, a dekafin, a hexafin, or combinations thereof.

FGFR3 can be activated by at least FGF1, FGF2, FGF4, FGF5, FGF6, FGF8, FGF9, FGF16, FGF17, FGF18, FGF20, FGF9, FGF19, FGF21, and FGF23, and FGF17. Mutations in FGFR3 have been associated with defects in chrondrocyte proliferation and calcification, as well as achondroplasia. The FGFR3 agonist can be an FGFR3 agonistic antibody, an FGF polypeptide or a functional fragment thereof, FGF17 or a functional fragment thereof, PF-05231023, SUN11602, a dekafin, a hexafin, or combinations thereof.

FGFR4 can be activated by at least FGF1, FGF2, FGF4, FGF6, FGF7, FGF8, FGF9, FGF16, FGF17 and FGF18. The FGFR4 agonist can be an FGFR4 agonistic antibody, an FGF polypeptide or a functional fragment thereof, FGF17 or a functional fragment thereof, PF-05231023, SUN11602, a dekafin, a hexafin, or combinations thereof.

The FGFR agonist can be a member of the FGF8 subfamily. The FGFR agonist can be an FGFR1 agonist. The FGFR agonist can be FGF17. The FGF17 polypeptide can be at least about 90%, 95%, 97%, 98%, 99%, or 100% identical to SEQ ID NO: 54. The FGF17 can be 100% identical to SEQ ID NO: 54. The FGF17 polypeptide can be at least about 90%, 95%, 97%, 98%, 99%, or 100% identical to SEQ ID NO: 55. The FGF17 polypeptide can be 100% identical to SEQ ID NO: 55. The FGF17 polypeptide can be at least about 90%, 95%, 97%, 98%, 99%, or 100% identical to SEQ ID NO: 56. The FGF17 can be 100% identical to SEQ ID NO: 56. The FGF17 polypeptide can be at least about 90%, 95%, 97%, 98%, 99%, or 100% identical to SEQ ID NO: 57, wherein the sequence comprises the R204Q and K207Q mutations. In certain embodiments, the FGF17 polypeptide is 100% identical to SEQ ID NO: 57. In certain embodiments, the FGF17 polypeptide is at least about 90%, 95%, 97%, 98%, 99%, or 100% identical to SEQ ID NO: 58. The FGF17 polypeptide can be 100% identical to SEQ ID NO: 58. The FGF17 polypeptide can be at least about 90%, 95%, 97%, 98%, 99%, or 100% identical to SEQ ID NO: 59, wherein the sequence comprises the K191A, K193A, and S200A mutations.

The FGF17 polypeptide can be 100% identical to SEQ ID NO: 59.

Glycosaminoglycans are linear polysaccharides containing repeating disaccharide units. There are four glasses of glycosaminoglycans: heparin/heparin sulfate, chondroitin sulfate/dermatan sulfate, keratin sulfate, and hyaluronic acid. The glycosaminoglycan can be a heparin/heparin sulfate, a chondroitin sulfate/dermatan sulfate, a keratin sulfate, or a hyaluronic acid. In some embodiments, the glycosaminoglycan comprises a heparin. The glycosaminoglycan can comprise a hyaluronic acid.

Heparin can be derived from natural sources, often referred to as unfractionated heparin. Heparin can also be defined based upon molecular weight. Low molecular weight heparin includes dalteparin, enoxaparin, certoparin, ardeparin, parnaparin, reviparin, nadroparin, and danaparoid. Heparin can be administered in a mixture with other compounds, such as danaparoid, which is a mixture of heparan sulfate, dermatan sulfate, and chrondriotin sulfate. The composition can comprise low molecular weight heparin, heparin sulfate, unfractionated heparin, heparin tetrasaccharide, dalteparin, tinzaparin, enoxaparin, certoparin, ardeparin, parnaparin, reviparin, nadroparin, heparin flush, danaparoid, fondaparinux, or combinations thereof.

Hyaluronic acid (HA) is a polymeric molecule and can exhibit a range of molecular weights. Hyaluronic acid can be used at almost any average of modal molecular weight formulation of The molecular weight can be, for example, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 kDa or more, or any range derivable therein. HA can include low molecular weight HA (about 500 to 700 kilodaltons kDa), medium molecular weight HA (700-1000 kDa), and high molecular weight HA (1.0-4.0 million daltons (MDa)). HA includes natural formulations, synthetic formulations, or combinations thereof. In some embodiments, the HA is a low molecular weight, a medium molecular weight, a low molecular weight, or a combination thereof. In some embodiments, the HA is a natural HA, a synthetic HA, or a combination thereof.

The HA may be a hyaluronic acid derivative. Examples of chemical modifications which may be made to HA include any reaction of an agent with the four reactive groups of HA, namely the acetamido, carboxyl, hydroxyl, and the reducing end. HA derivatives include, without limitations, hydrophobized hyaluronan, maleimide modified HA, methacrylated hyaluronic acid, or a sulfated hyaluronic acid. In some embodiments, the HA is modified at an acetamido group, a carboxyl group, a hydroxyl group, a reducing end, or combinations thereof. The HA can comprise a hydrophobized hyaluronan, a maleimide modified HA, a methacrylated hyaluronic acid, a sulfated hyaluronic acid, or a combination thereof. The HA can be covalently cross-linked via proteins or organic molecules into higher molecular weight moieties.

Also described herein are methods comprising administering an FGFR agonist and a glycosaminoglycan. The administration can be in the same composition, separate formulations. When separate formulations are administered they can be administered effectively simultaneously (e.g., during the same treatment) or separately with an interval of at least 1 hour, 12 hours, 24 hours, 2 days, 3 days, 4 days, 5 days, 6 days, 7 days, or more. When separate formulations are administered they can be administered by the same route or different routes selected from intravenous, intradermal, and subcutaneous. The formulations, whether separate or singular can be administered directly to the site of muscle or soft-tissue injury.

### Combinations of an IGF1R Agonist and a Short Fatty Acid Chain

In certain aspects, disclosed herein is a composition comprising an IGF1R agonist and a short fatty acid chain. IGF1R signaling activates downstream pathways including pathways involved in cell proliferation, cell differentiation, and cell survival. The two IGF ligands, IGF1 and IGF2, activate IGF1R signaling. Additional peptides that activate IGF1R signaling are INS. Other agonists of IGF1R include, without limitations, demethylasterriquinone B1, Ginsenoside Rg5, and the human antimicrobial peptide LL-37. T can be IGF1R agonist comprise an IGF1R agonistic antibody, an IGF polypeptide or a functional fragment thereof, IGF2 or a functional fragment thereof, insulin, demethylasterriquinone B1, Ginsenoside Rg5, LL-37, or combinations thereof. These compositions comprise an unexpected synergistic effect and are useful for treating the muscle and/or soft-tissue conditions or disorders. This synergistic effect may also be promoted by methods comprising separate administration of an IGF1R agonist and a short fatty acid chain.

The IGF2R agonist can be an IGF ligand. The IGF1R agonist can be IGF2. The IGF2 polypeptide can be at least about 90%, 95%, 97%, 98%, 99%, or 100% identical to SEQ ID NO. 76. The IGF2 polypeptide can be 100% identical to SEQ ID NO. 76. The IGF2 polypeptide can be at least about 90%, 95%, 97%, 98%, 99%, or 100% identical to SEQ ID NO. 80. The IGF2 polypeptide can be 100% identical to SEQ ID NO. 80. The IGF2 polypeptide can be at least about 90%, 95%, 97%, 98%, 99%, or 100% identical to SEQ ID NO. 81. The IGF2 polypeptide can be 100% identical to SEQ ID NO. 81.

The composition can comprise an IGF1R agonist and a short fatty acid chain. Short fatty acid chains include, without limitations, butyrates, a phenylbutyrate, valproic acid, propionic acid, methanoic acid, ethanoic acid, 2-methylpropanoic acid, 3-methylbutanoic acid, pentanoic acid, and a multimerized version thereof such as tributyrin. Butyrates include, without limitations, butyric acids, sodium butyrate, methyl butyrate, ethyl butyrate, butyl butyrate, pentyl butyrate, or sodium butyrate. The short chain fatty acid can be a butyrate. The butyrate can be butyric acid. The butyrate can be sodium butyrate. The short chain fatty acid can be a phenylbutyrate, valproic acid, propionic acid, methanoic acid, ethanoic acid, 2-methylpropanoic acid, 3-methylbutanoic acid, pentanoic acid, or a multimerized version thereof such as tributyrin.

Also described herein are methods comprising administering an IGF1R agonist and a short fatty acid chain. The administration can be in the same composition, separate formulations. When separate formulations are administered, they can be administered effectively simultaneously (e.g., during the same treatment) or separately with an interval of at least 1 hour, 12 hours, 24 hours, 2 days, 3 days, 4 days, 5 days, 6 days, 7 days, or more.

Combinations of BMP Receptor Agonists and mTOR Activators

In certain aspects, disclosed herein is a composition comprising a BMP receptor agonist and an mTOR activator. BMP receptors activate downstream signaling through the TGF-beta pathway and are involved in many cell functions including differentiation, proliferation, and migration. There are two classes of BMP receptors: BMP type I (ACVR1, BMPR1A, and BMPR1B), and BMP type II (BMP2R, ACVR2A, and ACVR2B). BMP type 1 receptors bind BMP ligands exclusively, while BMP type II receptors bind BMPs and related proteins, including activin, Gdf9, and GDf11. The BMP receptor agonist can comprise an ACVR1 agonist, a BMPR1A agonist, a BMPR1B agonist, a BMP2R agonist, an ACVR2A agonist, an ACVR2B agonist, or a combination thereof. The BMP receptor agonist can comprise an ACVR1 agonist, an ACVR2A agonist, an ACVR2B agonist, a BMPR1A agonist, or a combination thereof. The BMP receptor can comprise an ACVR1 agonist. The BMP receptor agonist can comprise an ACVR2A agonist. The BMP receptor can comprise an ACRV2B agonist. The BMP receptor can comprise a BMPR1A agonist. The BMP receptor agonist can comprise an ACVR1 agonist antibody, a BMPR1A agonist antibody, a BMPR1B agonist antibody, a BMP2R agonist antibody, a ACVR2A agonist antibody, a ACVR2B agonist antibody, a BMP polypeptide or a functional fragment thereof, a BMP7 or a functional fragment thereof, ventromorphin, SB4, tacrolimus, isoliquiritigeni, alantolactone, PD407824, or combinations thereof. These compositions comprise an unexpected synergistic effect and are useful for treating the muscle and/or soft-tissue conditions or disorders. This synergistic effect may also be promoted by methods comprising separate administration of a BMP receptor agonist and a leucine.

The BMP7 polypeptide can be at least about 90%, 95%, 97%, 98%, 99%, or 100% identical to SEQ ID NO: 89. The BMP7 polypeptide can be 100% identical to SEQ ID NO: 89. The BMP7 polypeptide can be at least about 90%, 95%, 97%, 98%, 99%, or 100% identical to SEQ ID NO: 90. The BMP7 polypeptide can be 100% identical to SEQ ID NO: 90. The BMP7 polypeptide can be at least about 90%, 95%, 97%, 98%, 99%, or 100% identical to SEQ ID NO: 91. The BMP7 polypeptide can be 100% identical to SEQ ID NO: 91. The BMP7 polypeptide can be at least about 90%, 95%, 97%, 98%, 99%, or 100% identical to SEQ ID NO: 93. The BMP7 polypeptide can be 100% identical to SEQ ID NO: 93.

The mammalian target of rapamycin (mTOR) pathway is a key regulator of skeletal muscle mass and growth. mTOR is a serine/threonine kinase involved in diverse cellular processes including cell growth, differentiation, autophagy, survival, and metabolism. mTOR activation through the mTORC1 complex is required both for myofibrillar muscle protein synthesis and skeletal muscle hypertrophy. Inactivation of the mTORC1 complex has been found to be associated with loss of muscle mass and muscle struct during muscle wasting due to old age, cachexia, and atrophy due to physical activity.

The mTOR signaling pathway can be activated by many different signals, including amino acids, polypeptides, and small molecules. The mTOR activator can be an amino acid. The amino acid can be leucine, valine, isoleucine or a combination thereof. The amino acid can be leucine. The mTOR activator can be a polypeptide. The polypeptide can comprise a Ras homolog enriched in brain (Rheb), tuberous sclerosis complex (TSC), protein kinase B (PKB), extracellular-signal-regulated kinase ½ (ERK1/2), p90 ribosomal s6 kinase 1 (RSK1), Wnt ligands, or a combination thereof. The mTOR activator can be a small molecule. The mTOR activator can comprise MHY1485, NV-5138, 3-benzyl-5-((2-nitrophenoxy) methyl)-dihydrofuran-2(3H)-one (3BDO), or a combination thereof. The mTOR activator can comprise leucine, valine, isoleucine, Ras homolog enriched in brain (Rheb), tuberous sclerosis complex (TSC), protein kinase B (PKB), extracellular-signal-regulated kinase ½ (ERK1/2), p90 ribosomal s6 kinase 1 (RSK1), a Wnt ligand, MHY1485, NV-5138, 3-benzyl-5-((2-nitrophenoxy) methyl)-dihydrofuran-2(3H)-one (3BDO), or a combination thereof.

The mTOR activator can comprise leucine. The amino acid leucine is an important part of mTOR signaling within skeletal muscle. Leucine is a branched chain amino acid that is essential to the human diet. It is the single most common amino acid in human proteins, at a frequency of nearly 1 in 10 amino acids (UniProtKB/Swiss-Prot release 2013_04—April 2013). The circulating and intracellular concentrations of this amino acid are monitored and tightly controlled as part of feedback mechanisms controlling major anabolic and catabolic processes such as cell division, protein synthesis, and autophagy. Part of the regulatory effects of leucine are mediated by the mTORC1 complex whose activation to drive protein synthesis and cell cycle progression are in part driven by intracellular leucine concentration. The combination of a BMP receptor agonist and leucine or another branched chain amino acid demonstrate synergistical mitogenic activity.

Synthetic leucine derivatives may be used. The leucine can comprise 1-leucine, glycyl-1-leucine, acetyl-1-leucine, 1-leucine ethyl ester, and 1-leucine methyl ester, caproic acid, phthaloyl-1-leucine, benzoyl-dl-leucine, or a combination thereof. The leucine can be a salt. The leucine can comprise 1-leucenium hydrogen maleate, leucine hydrochloride, or a combination thereof.

Also described herein are methods comprising administering a BMP receptor agonist and an mTOR activator. The administration can be in the same composition, or separate formulations. When separate formulations are administered, they can be administered effectively simultaneously (e.g., during the same treatment) or separately with an interval of at least 1 hour, 12 hours, 24 hours, 2 days, 3 days, 4 days, 5 days, 6 days, 7 days, or more.

### Therapeutic Indications

In certain aspects, the fusion polypeptides comprising an FGF8 subfamily amino acid sequence and a heterologous polypeptide, compositions comprising an FGFR agonist and a glycosaminoglycan, and the methods described herein, are useful for treating diseases and disorders that involve soft-tissue injury, degradation, or destruction, or for use in treating an individual with an aging disorder, a muscle wasting disorder, a muscle injury, an injury to a connective tissue, or an injury to a non-muscle soft-tissue, or any combination thereof.

In certain aspects, the fusion polypeptides comprising an IGF ligand amino acid sequence and a heterologous polypeptide, compositions comprising an IGF1R agonist and a short fatty acid chain, and the methods described herein, are useful for treating diseases and disorders that involve soft-tissue injury, degradation, or destruction, or for use in treating an individual with an aging disorder, a muscle wasting disorder, a muscle injury, an injury to a connective tissue, or an injury to a non-muscle soft-tissue, or any combination thereof.

In certain aspects, the fusion polypeptides comprising a BMP7 amino acid sequence, compositions comprising a BMP receptor agonist and a glycosaminoglycan, compositions comprising a BMP receptor agonist and an mTOR activator, and the methods, described herein, are useful for treating diseases and disorders that involve soft-tissue injury, degradation, or destruction, or for use in treating an individual with an aging disorder, a muscle wasting disorder, a muscle injury, an injury to a connective tissue, or an injury to a non-muscle soft-tissue, or any combination thereof.

Aging disorders that result in the deterioration and loss of muscle tissue are such disorders. Sarcopenia, for example, is the degenerative loss of skeletal muscle mass quality, and strength and can be associated with aging. Injuries that result in acute muscle damage are other muscle disorders, which are treatable by the polypeptides, compositions and methods described herein. The disorders include muscle ruptures, strains, and contusions. A rupture is a separating of the muscle tissues. Muscle strains are contraction-induced injuries in which muscle fibers tear due to extensive mechanical stress, and can be classified as a grade I, II, or III. Muscle contusions are muscle hematomas. Muscle injury can also be caused by non-mechanical stresses such as cachexia. Cachexia may be caused by malnutrition, cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), Crohn's disease, untreated/severe type 1 diabetes mellitus, anorexia nervosa, chemotherapy, muscular dystrophy or other genetic diseases which cause immobility, and hormonal deficiencies. Certain disorders that are weaknesses of specific muscles such as dysphagia or facioscapulohumeral muscular dystrophy may also be treated by the polypeptides described herein. Additional soft-tissues disorders that may be treated using the polypeptides comprising an FGF8 subfamily amino acid sequence and/or compositions comprising an FGFR agonist and a glycosaminoglycan described herein are those that inflict injury to the tendons, ligaments or cartilage. Additional soft-tissues disorders that may be treated using the polypeptides comprising an IGF ligand amino acid sequence and compositions comprising an IGF1R agonist and a short fatty acid chain described herein are those that inflict injury to the tendons, ligaments or cartilage. Additional soft-tissues disorders that may be treated using the fusion polypeptides comprising a BMP7 amino acid sequence, compositions comprising a BMP receptor agonist and a glycosaminoglycan, and the methods, described herein, are those that inflict injury to the tendons, ligaments or cartilage.

The muscle wasting disease can be a muscular dystrophy. The muscular dystrophy can comprise a myotonic muscular dystrophy, Duchenne muscular dystrophy, Becker muscular dystrophy, Limb-girdle muscular dystrophy, facioscapulohumeral muscular dystrophy, congenital, muscular dystrophy, oculopharyngeal muscular dystrophy, or distal muscular dystrophy. The muscular dystrophy can be myotonic dystrophy.

The aging disorder can be sarcopenia. The muscle wasting disorder can be cachexia. The cachexia can be a result of a cancer, AIDS, end stage kidney disease, or cardiovascular disease. The injury can be a muscle injury. The muscle wasting can be atrophy due to limb immobilization or disuse. The muscle injury can be a strain or a tear. The muscle injury can be a Grade III strain. Sarcopenia can contribute to the incidence of the muscle injury. The injury can be ligament damage. The ligament damage can be a rupture or a tear. The injury can be tendon damage. The tendon damage can be a rupture or a tear. The injury can be cartilage damage.

The compositions described herein, are for use in a method of treating myositis. The myositis can comprise dermatomyositis, polymyositis, necrotizing myopathy (also called necrotizing autoimmune myopathy or immune-mediated necrotizing myopathy), juvenile myositis, or sporadic inclusion-body myositis.

The compositions described herein can be for use in a method of treating cartilage related-disorders. The cartilage related disorder may be due to tears, injuries, or wear. The cartilage-associated disease may be osteoarthritis, osteochondritis dissecans, achondroplasia, or degenerative cartilage lesions.

The compositions described herein can be for use in a method of increasing proliferation or promoting survival of a cell associated with soft-tissue damage. The polypeptides comprising an IGF ligand amino acid sequence and compositions comprising an IGF1R agonist and a short fatty acid chain described herein can be useful in a method of increasing proliferation or promoting survival of any one or more of a muscle cell, a muscle precursor cell, a tenocyte, a tenocyte precursor cell, a chondrocyte, a chondrocyte precursor cell, a mesenchymal stem cell, or a fibroblast.

Muscle fibrosis is an excessive accumulation of extracellular matrix components, including collagen. Muscle fibrosis impairs muscle function, negatively affects muscle regeneration after injury, and increases muscle susceptibility to re-injury. The compositions described herein can be for use in a method of reducing muscle fibrosis. The fibrosis can be associated with aging, muscular dystrophy, or an injury. The IGF ligand can be IGF2.

In order to differentiate into mature muscle cells, myoblasts must fuse and form multinucleated cells. In certain embodiments, the fusion polypeptides comprising an IGF ligand amino acid sequence and a heterologous polypeptide, compositions comprising an IGF1R agonist and a short fatty acid chain, and the methods described herein are for use in a method of increasing myoblast fusion. The IGF ligand can be IGF2.

The fusion polypeptides comprising an IGF ligand amino acid sequence and a heterologous polypeptide, compositions comprising an IGF1R agonist and a short fatty acid chain, and the methods described herein can be for use in a method of increasing muscle mass. Muscle mass can be increased by at least about 1%, 2.5%, 5%, 10%, 20%, 30%, 40%, 50% or more than 50%. The IGF ligand can be IGF2.

The fusion polypeptides comprising an IGF ligand amino acid sequence and a heterologous polypeptide, compositions comprising an IGF1R agonist and a short fatty acid chain, and the methods described herein can be for use in a method of increasing grip strength. Grip strength can be increased by at least about 1%, 2.5%, 5%, 10%, 20%, 30%, 40%, 50% or more than 50%. The IGF ligand can be IGF2.

The fusion polypeptides comprising an IGF ligand amino acid sequence and a heterologous polypeptide, compositions comprising an IGF1R agonist and a short fatty acid chain, and the methods described herein can be for use in a method of increasing muscle endurance. Muscle endurance can be increased by at least about 1%, 2.5%, 5%, 10%, 20%, 30%, 40%, 50% or more than 50%. The IGF ligand can be IGF2.

### Methods of Treatment

In certain aspects, disclosed herein is a method of treating an individual with a disorder comprising administering an FGFR agonist and a glycosaminoglycan to the individual with the disorder. The FGFR agonist and the glycosaminoglycan can be administered in separate formulations. The FGFR agonist and the glycosaminoglycan can be administered simultaneously. The FGFR agonist and the glycosaminoglycan can be administered at different times. The glycosaminoglycan can be a heparin. The glycosaminoglycan can be a hyaluronic acid.

In certain aspects, disclosed herein is a method of treating an individual with a disorder comprising administering an FGFR1 agonist and a glycosaminoglycan to the individual the disorder. The FGFR1 agonist and the glycosaminoglycan (e.g., heparin) can be administered in separate formulations. The FGFR1 agonist and the glycosaminoglycan (e.g., heparin) can be administered simultaneously. The FGFR1 agonist and the glycosaminoglycan (e.g., heparin) can be administered at different times. The glycosaminoglycan can be a heparin. The glycosaminoglycan can be a hyaluronic acid.

In certain aspects, disclosed herein is a method of treating an individual with a disorder comprising administering a polypeptide comprising a FGF8 subfamily amino acid sequence and a glycosaminoglycan (e.g., heparin) or a compound or mixture comprising a glycosaminoglycan to the individual the disorder. The polypeptide comprising a FGF8 subfamily amino acid sequence and the glycosaminoglycan (e.g., heparin) can be administered in separate formulations. The polypeptide comprising a FGF8 subfamily amino acid sequence and the glycosaminoglycan (e.g., heparin) can be administered simultaneously. The polypeptide comprising a FGF8 subfamily amino acid sequence and the glycosaminoglycan (e.g., heparin) can be administered at different times. The glycosaminoglycan can be a hyaluronic acid.

In certain aspects, disclosed herein is a method of treating an individual with a disorder comprising administering a polypeptide comprising a FGF17 amino acid sequence and a glycosaminoglycan (e.g., heparin) or a compound or mixture comprising glycosaminoglycan to the individual the disorder. The polypeptide comprising a FGF17 amino acid sequence and the glycosaminoglycan (e.g., heparin) can be administered in separate formulations. The polypeptide comprising a FGF17 amino acid sequence and the glycosaminoglycan (e.g., heparin) can be administered simultaneously. The polypeptide comprising a FGF17 amino acid sequence and the glycosaminoglycan can be administered at different times. The glycosaminoglycan can be a hyaluronic acid.

In certain aspects, disclosed herein is a method of treating an individual with a disorder comprising administering a polypeptide comprising an FGF8 subfamily amino acid sequence. The polypeptide can comprise a FGF17 amino acid sequence. The polypeptide can comprise a FGF17 fusion protein.

In certain aspects, disclosed herein is a method of treating an individual with a disorder comprising administering an IGF1R agonist and a short fatty acid chain (e.g., butyrate) to the individual. The IGF1R agonist and the short fatty acid chain (e.g., butyrate) can be administered in separate formulations. The IGF1R agonist and the short fatty acid chain (e.g., butyrate) can be administered simultaneously. The IGF1R agonist and the short fatty acid chain (e.g., butyrate) can be administered at different times.

In certain aspects, disclosed herein is a method of treating an individual with a disorder comprising administering a polypeptide comprising an IGF ligand amino acid sequence and a butyrate to the individual the disorder. The polypeptide comprising the IGF ligand amino acid sequence and the butyrate can be administered in separate formulations. The polypeptide comprising the IGF ligand amino acid sequence and the butyrate can be administered simultaneously. The polypeptide comprising the IGF ligand amino acid sequence and the butyrate can be administered at different times.

In certain aspects, disclosed herein is a method of treating an individual with a disorder comprising administering a polypeptide comprising an IGF2 amino acid sequence and a short fatty acid chain (e.g., butyrate) to the individual the disorder. The polypeptide comprising the IGF ligand amino acid sequence and the short fatty acid chain (e.g., butyrate) can be administered in separate formulations. The polypeptide comprising the IGF2 amino acid sequence and the short fatty acid chain (e.g., butyrate) can be administered simultaneously. The polypeptide comprising the IGF2 amino acid sequence and the short fatty acid chain (e.g., butyrate) can be administered at different times.

In certain aspects, disclosed herein is a method of treating an individual with a disorder comprising administering a BMP receptor agonist and a glycosaminoglycan to the individual the disorder. The BMP receptor agonist and a glycosaminoglycan can be administered in separate formulations. The BMP receptor agonist and a glycosaminoglycan can be administered simultaneously. The BMP receptor agonist and a glycosaminoglycan can be administered at different times. The glycosaminoglycan can be a heparin. The glycosaminoglycan can be a hyaluronic acid.

In certain aspects, disclosed herein is a method of treating an individual with a disorder comprising administering a polypeptide comprising a BMP7 amino acid sequence and hyaluronic acid or a compound or mixture comprising hyaluronic acid to the individual the disorder. The polypeptide comprising a BMP7 amino acid sequence and the hyaluronic acid are administered in separate formulations. In some embodiments, the polypeptide comprising a BMP7 amino acid sequence and the hyaluronic acid are administered simultaneously. In some embodiments, the polypeptide comprising a BMP7 amino acid sequence and the hyaluronic acid are administered at different times.

In certain aspects, disclosed herein is a method of treating an individual with a disorder comprising administering a polypeptide comprising a BMP7 amino acid sequence and heparin or a compound or mixture comprising heparin to the individual the disorder. In some embodiments, the polypeptide comprising a BMP7 amino acid sequence and the heparin are administered in separate formulations. In some embodiments, the polypeptide comprising a BMP7 amino acid sequence and the heparin are administered simultaneously. In some embodiments, the polypeptide comprising a BMP7 amino acid sequence and the heparin are administered at different times.

In certain aspects, disclosed herein is a method of treating an individual with a disorder comprising administering a BMP receptor agonist and an mTOR activator to the individual the disorder. In some embodiments, the BMP receptor agonist and an mTOR activator are administered in separate formulations. In some embodiments, the BMP receptor agonist and an mTOR activator are administered simultaneously. In some embodiments, the BMP receptor agonist and an mTOR activator are administered at different times. In certain embodiments, mTOR activator is a leucine.

In certain aspects, disclosed herein is a method of treating an individual with a disorder comprising administering a polypeptide comprising a BMP7 amino acid sequence and leucine or a compound or mixture comprising leucine to the individual the disorder. In some embodiments, the polypeptide comprising a BMP7 amino acid sequence and the leucine are administered in separate formulations. In some embodiments, the polypeptide comprising a BMP7 amino acid sequence and the leucine are administered simultaneously. In some embodiments, the polypeptide comprising a BMP7 amino acid sequence and the leucine are administered at different times.

In certain aspects, disclosed herein is a method of treating an individual with a disorder comprising administering a BMP receptor agonist and a glycosaminoglycan to the individual the disorder. In some embodiments, the BMP receptor agonist and a glycosaminoglycan are administered in separate formulations. In some embodiments, the BMP receptor agonist and a glycosaminoglycan are administered simultaneously. In some embodiments, the BMP receptor agonist and a glycosaminoglycan are administered at different times. In certain embodiments, the glycosaminoglycan is a heparin. In certain embodiments, the glycosaminoglycan is a hyaluronic acid.

In certain aspects, disclosed herein is a method of treating an individual with a disorder comprising administering a polypeptide comprising a BMP7 amino acid sequence and a glycosaminoglycan (e.g., heparin or hyaluronic acid) or a compound or mixture comprising a glycosaminoglycan (e.g., heparin or hyaluronic acid) to the individual the disorder. In some embodiments, the polypeptide comprising a BMP7 amino acid sequence and the glycosaminoglycan (e.g., heparin or hyaluronic acid) are administered in separate formulations. In some embodiments, the polypeptide comprising a BMP7 amino acid sequence and the glycosaminoglycan (e.g., heparin or hyaluronic acid) are administered simultaneously. In some embodiments, the polypeptide comprising a BMP7 amino acid sequence and the glycosaminoglycan (e.g., heparin or hyaluronic acid) are administered at different times.

The treatment can be administered by any suitable route such as, for example, subcutaneous, intravenous, or intramuscular. In certain embodiments, the treatment is administered on a suitable dosage schedule, for example, weekly, twice weekly, monthly, twice monthly, once every three weeks, or once every four weeks. The treatment can be administered in any therapeutically effective amount. The therapeutically effective amount can be about 0.001 mg/kg to about 1 mg/kg. The therapeutically effective amount can be about 0.001 mg/kg to about 0.002 mg/kg, about 0.001 mg/kg to about 0.005 mg/kg, about 0.001 mg/kg to about 0.01 mg/kg, about 0.001 mg/kg to about 0.02 mg/kg, about 0.001 mg/kg to about 0.05 mg/kg, about 0.001 mg/kg to about 0.1 mg/kg, about 0.001 mg/kg to about 0.2 mg/kg, about 0.001 mg/kg to about 0.5 mg/kg, about 0.001 mg/kg to about 1 mg/kg, about 0.002 mg/kg to about 0.005 mg/kg, about 0.002 mg/kg to about 0.01 mg/kg, about 0.002 mg/kg to about 0.02 mg/kg, about 0.002 mg/kg to about 0.05 mg/kg, about 0.002 mg/kg to about 0.1 mg/kg, about 0.002 mg/kg to about 0.2 mg/kg, about 0.002 mg/kg to about 0.5 mg/kg, about 0.002 mg/kg to about 1 mg/kg, about 0.005 mg/kg to about 0.01 mg/kg, about 0.005 mg/kg to about 0.02 mg/kg, about 0.005 mg/kg to about 0.05 mg/kg, about 0.005 mg/kg to about 0.1 mg/kg, about 0.005 mg/kg to about 0.2 mg/kg, about 0.005 mg/kg to about 0.5 mg/kg, about 0.005 mg/kg to about 1 mg/kg, about 0.01 mg/kg to about 0.02 mg/kg, about 0.01 mg/kg to about 0.05 mg/kg, about 0.01 mg/kg to about 0.1 mg/kg, about 0.01 mg/kg to about 0.2 mg/kg, about 0.01 mg/kg to about 0.5 mg/kg, about 0.01 mg/kg to about 1 mg/kg, about 0.02 mg/kg to about 0.05 mg/kg, about 0.02 mg/kg to about 0.1 mg/kg, about 0.02 mg/kg to about 0.2 mg/kg, about 0.02 mg/kg to about 0.5 mg/kg, about 0.02 mg/kg to about 1 mg/kg, about 0.05 mg/kg to about 0.1 mg/kg, about 0.05 mg/kg to about 0.2 mg/kg, about 0.05 mg/kg to about 0.5 mg/kg, about 0.05 mg/kg to about 1 mg/kg, about 0.1 mg/kg to about 0.2 mg/kg, about 0.1 mg/kg to about 0.5 mg/kg, about 0.1 mg/kg to about 1 mg/kg, about 0.2 mg/kg to about 0.5 mg/kg, about 0.2 mg/kg to about 1 mg/kg, or about 0.5 mg/kg to about 1 mg/kg. The therapeutically effective amount can be about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, or about 1 mg/kg. The therapeutically effective amount can be at least about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, or about 0.5 mg/kg. The therapeutically effective amount can be at most about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, or about 1 mg/kg. The therapeutically effective amount can be about 0.1 mg/kg to about 50 mg/kg. The therapeutically effective amount can be about 0.1 mg/kg to about 0.2 mg/kg, about 0.1 mg/kg to about 0.5 mg/kg, about 0.1 mg/kg to about 1 mg/kg, about 0.1 mg/kg to about 2 mg/kg, about 0.1 mg/kg to about 5 mg/kg, about 0.1 mg/kg to about 10 mg/kg, about 0.1 mg/kg to about 20 mg/kg, about 0.1 mg/kg to about 50 mg/kg, about 0.2 mg/kg to about 0.5 mg/kg, about 0.2 mg/kg to about 1 mg/kg, about 0.2 mg/kg to about 2 mg/kg, about 0.2 mg/kg to about 5 mg/kg, about 0.2 mg/kg to about 10 mg/kg, about 0.2 mg/kg to about 20 mg/kg, about 0.2 mg/kg to about 50 mg/kg, about 0.5 mg/kg to about 1 mg/kg, about 0.5 mg/kg to about 2 mg/kg, about 0.5 mg/kg to about 5 mg/kg, about 0.5 mg/kg to about 10 mg/kg, about 0.5 mg/kg to about 20 mg/kg, about 0.5 mg/kg to about 50 mg/kg, about 1 mg/kg to about 2 mg/kg, about 1 mg/kg to about 5 mg/kg, about 1 mg/kg to about 10 mg/kg, about 1 mg/kg to about 20 mg/kg, about 1 mg/kg to about 50 mg/kg, about 2 mg/kg to about 5 mg/kg, about 2 mg/kg to about 10 mg/kg, about 2 mg/kg to about 20 mg/kg, about 2 mg/kg to about 50 mg/kg, about 5 mg/kg to about 10 mg/kg, about 5 mg/kg to about 20 mg/kg, about 5 mg/kg to about 50 mg/kg, about 10 mg/kg to about 20 mg/kg, about 10 mg/kg to about 50 mg/kg, or about 20 mg/kg to about 50 mg/kg. The therapeutically effective amount can be about 0.1 mg/kg, about 0.2 mg/kg, about 0.5 mg/kg, about 1 mg/kg, about 2 mg/kg, about 5 mg/kg, about 10 mg/kg, about 20 mg/kg, or about 50 mg/kg. The therapeutically effective amount can be at least about 0.1 mg/kg, about 0.2 mg/kg, about 0.5 mg/kg, about 1 mg/kg, about 2 mg/kg, about 5 mg/kg, about 10 mg/kg, or about 20 mg/kg. The therapeutically effective amount can be at most about 0.2 mg/kg, about 0.5 mg/kg, about 1 mg/kg, about 2 mg/kg, about 5 mg/kg, about 10 mg/kg, about 20 mg/kg, or about 50 mg/kg.

The individual treated can be a mammal. The mammal can be a mouse, rat, rabbit, dog, cat, horse, cow, sheep, pig, goat, llama, alpaca, or yak. The individual can be a dog, cat, or a horse. The individual to be treated can be a human.

### Methods of Production

The polypeptide comprising an FGF17, IGF, or BMP7 ligand amino acid sequence can be purified or synthesized in any suitable manner. A nucleic acid encoding the polypeptide can be cloned into a suitable vector and expressed in a suitable cellular system. The cellular system can be a prokaryotic cell system. The cellular system can be a eukaryotic cell system. The cellular system can be a mammalian cell system. The polypeptide may be expressed from Eschericia coli. The polypeptide may be expressed from a yeast cell, including without limitations, Saccharomyces cerevisiae, Pichia pastoris, Kluyveromyces lactis, Hansenula polymorpha, or Yarrowia lipolytica. The polypeptide may be expressed from a mouse myeloma cell, including without limitations, NS0, Sp2/0, and FO. The polypeptide may be expressed from a chinese hamster ovary (CHO) cell. The polypeptide may be expressed by a mammalian cell, including without limitations, a COS cell, a Vero cell, or a BHK cell. The polypeptide may be expressed from a human cell, including without limitations a HeLa cell, a HEK-293 cell, a CAP cell, a CAP-T cell, a PER.C6® cell.

The supernatants from such an expression system can be subjected to one or more purification steps involving centrifugation, ultracentrifugation, filtration, diafiltration, tangential-flow filtration, dialysis, chromatography (e.g., cation exchange, ion exchange, hydrophobic interaction, reverse phase, affinity, or size exclusion). The polypeptides can be purified to an extent suitable for human administration. Additionally, polypeptides can be synthesized for inclusion in a formulation to be administered to a human individual. The polypeptides can be produced by a suitable peptide synthesis method, such as solid-phase synthesis.

The mammalian expression vector pmax Cloning can be used to make C-terminally 6×His-tagged, StrepII-tagged, and human IgG1 Fc-tagged vectors. The DNA fragments encoding the secreted myogenic factors are amplified by PCR from human open reading frame (ORF) clones, and subsequently inserted into the tagged vectors by In-Fusion cloning technology (Takara Bio Inc.). The expression vectors carrying the secreted myogenic factors are transiently transfected into ExpiCHO-S cells at a density of 6×10⁶ per ml by using ExpiFectamine CHO transfection kit (Thermo Scientific).

The expressed myogenic factors with different tags in the culture supernatants are affinity-purified by using different purification media. The polypeptide can comprise an Fc region. For these polypeptides a matrix or resin comprising Protein A, Protein G, protein L or any combination thereof can be used. The matrix or resin may suitably be loaded onto a column for ease in batch purification.

### Purification of Immunoglobulin Fusion Proteins

The heterologous sequence may comprise an immunoglobulin or a fragment thereof. When the polypeptide comprises an immunoglobulin or a fragment thereof, the polypeptide may be purified by means of protein A, G, or L affinity. Protein A and G are cell surface proteins found in Staphylococcus aureus. They have the property of binding the Fc region of a mammalian antibody, in particular of IgG class antibodies. For use in protein A or G affinity chromatography, protein A or G is coupled to a solid matrix such as crosslinked, uncharged agarose (Sepharose, freed from charged fraction of natural agarose), trisacryl, crosslinked dextran or silica-based materials. Methods for such are commonly known in the art, e.g. coupling via primary amino functions of the protein to a CNBr-activated matrix. Protein A binds with high affinity and high specificity to the Fc portion of IgG, that is the Cγ2-Cγ3 interface region of IgG as described in Langone et al., 1982, supra. In particular, it binds strongly to the human allotypes or subclasses IgG1, IgG2, IgG3 and the mouse allotypes or subclasses IgG2a, IgG2b, IgG3.

After purification by Protein A, G, or L the bound fraction can be eluted and passed over or through an additional resin or matrix comprising one or more ion exchange columns. The first ion exchanger is generally an anion exchanger resin. The pH of buffer used for loading and running the first ion exchanger is set as to put opposing total change on the Fc comprising fusion polypeptide and the protein A to be separated by means of the ion exchanger in a flow-through mode according to the present invention, taking the pI's of the Fc comprising fusion polypeptide and protein A into account. The mode of operation of a first anion exchanger according to the present invention requires buffer exchange of the acidic or neutralized eluate from the protein A affinity chromatography step with the equilibrium buffer of the first anion exchanger. After the first anion exchanger, the Fc comprising fusion polypeptide is ready for use in applications or may be deemed to require further polishing by customary purification methods. In a further preferred embodiment, the first ion exchange step is followed by a second ion exchange step in which second step the antibody is loaded and bound by the second ion exchange medium and is eluted with a buffer other than the loading buffer, by means of increased salt and/or pH, as an essentially monomeric, non-aggregated antibody.

In the methods disclosed herein at least 70%, 80%, or 90% of the Fc comprising fusion polypeptide loaded onto the first ion exchanger can be recovered in the flow-through of the ion-exchanger.

### Master Cell Bank and Trangenic Cells

Described herein are master cell banks that can comprise a cell that comprises a nucleic acid encoding one or more IGF ligand or IGF2 fusion polypeptides integrated into its genome creating a transgenic cell-line. The master cell bank can comprise a plurality of cells that each comprise a nucleic acid encoding an IGF ligand or IGF2 fusion polypeptide. The nucleic acid can be maintained extrachromosomally on a plasmid or yeast artificial chromosome. The nucleic acid can be integrated into a chromosomal location. The cell can be a yeast cell. The yeast can be Pichia pastoris or Saccharomyces cerevisiae. The cell can be a mammalian cell. The mammalian cell can be a 293T cell or derivative thereof (e.g., 293T-Rex). The cell can be a bacterial cell.

The transgenic mammalian, yeast, or bacterial cell can be a master cell bank that comprises a cryopreservative suitable for freezing to at least about −80° or below. The master cell bank can comprise glycerol or DMSO at between about 10 and about 30%, and can be suitable for long-term storage at about −80° or below. The master cell bank can preserve a transgenic mammalian, yeast, or bacterial strain for at least 1, 2, 3, 4, 5, 6, 7, 8, 9, 10, or more years.

### Pharmaceutically Acceptable Excipients, Carriers, and Diluents

The polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein can be administered in a pharmaceutical composition that comprises one or more pharmaceutically acceptable excipients, carriers, or diluents. The exact components can differ based upon the preferred route of administration. The excipients used in a pharmaceutical composition can provide additional function to the polypeptide by making the polypeptide suitable for a particular route of administration (e.g., intravenous, topical, subcutaneous, or intramuscular), increasing polypeptide stability, increasing penetration of a desired tissue (e.g., muscle or skin), increasing residence time at particular site, increasing solubility, enhancing the efficacy of the polypeptide, and/or reducing inflammatory reactions coincident with administration.

The compositions can be included in a pharmaceutical composition with a solubilizing emulsifying, or dispersing agent. The solubilizing agent can allow high-concentration solutions of fusion polypeptides that exceed at least about 2 mg/mL, 5 mg/mL, 10 mg/mL, 15 mg/mL, or 20 mg/mL. Carbomers in an aqueous pharmaceutical composition serve as emulsifying agents and viscosity modifying agents. The pharmaceutically acceptable excipient can comprise or consist of a carbomer. The carbomer can comprise or consist of carbomer 910, carbomer 934, carbomer 934P, carbomer 940, carbomer 941, carbomer 1342, or combinations thereof. Cyclodextrins in an aqueous pharmaceutical composition serve as solubilizing and stabilizing agents. The pharmaceutically acceptable excipient can comprise or consist of a cyclodextrin. The cyclodextrin can comprise or consist of alpha cyclodextrin, beta cyclodextrin, gamma cyclodextrin, or combinations thereof. Lecithin in a pharmaceutical composition may serve as a solubilizing agent. The solubilizing agent can comprise or consist of lecithin. Poloxamers in a pharmaceutical composition serve as emulsifying agents, solubilizing agents, and dispersing agents. The pharmaceutically acceptable excipient can comprise or consist of a poloxamer. The poloxamer can comprise or consist of poloxamer 124, poloxamer 188, poloxamer 237, poloxamer 338, poloxamer 407, or combinations thereof. Polyoxyethylene sorbitan fatty acid esters in a pharmaceutical composition serve as emulsifying agents, solubilizing agents, surfactants, and dispersing agents. The pharmaceutically acceptable excipient can comprise or consist of a polyoxyethylene sorbitan fatty acid ester. The polyoxyethylene sorbitan fatty acid ester can comprise or consist of polysorbate 20, polysorbate 21, polysorbate 40, polysorbate 60, polysorbate 61, polysorbate 65, polysorbate 80, polysorbate 81, polysorbate 85, polysorbate 120, or combinations thereof. Polyoxyethylene stearates in a pharmaceutical composition serve as emulsifying agents, solubilizing agents, surfactants, and dispersing agents. The pharmaceutically acceptable excipient can comprise or consist of a polyoxyethylene stearate. The polyoxyethylene stearate can comprise or consist of polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, or combinations thereof. Sorbitan esters in a pharmaceutical composition serve as emulsifying agents, solubilizing agents, and non-ionic surfactants, and dispersing agents. The pharmaceutically acceptable excipient can comprise or consist of a sorbitan ester. The sorbitan ester can comprise or consist of sorbitan laurate, sorbitan oleate, sorbitan palmitate, sorbitan stearate, sorbitan trioleate, sorbitan sesquioleate, or combinations thereof. Solubility can be achieved with a protein carrier. The protein carrier can comprise recombinant human albumin.

The polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein can be formulated to increase stability. Polypeptides in aqueous formulations may require stabilization to prevent degradation. The stabilizer can comprise pH buffers, salts, amino acids, polyols/disaccharides/polysaccharides, liposomes, surfactants, antioxidants, reducing agents, or chelating agents. The stabilizer can comprise or consist of a polyol/non-reducing sugar. The non-reducing sugar can comprise or consist of sucrose, mannitol, trehalose, raffinose, stachyose, xylitol, starch, verbascose, or combinations thereof. Polypeptides can be encapsulated in liposomes to increase stability. The stabilizer can comprise or consist of liposomes. The liposomes can comprise or consist of ipalmitoylphosphatidylcholine (DPPC) liposomes, phosphatidylcholine:cholesterol (PC:Chol) (70:30) liposomes, or dipalmitoylphosphatidylcholine: dipalmitoylphosphatidylserine (DPPC:DPPS) liposomes (70:30). Non-ionic surfactants can increase the stability of a polypeptide. The stabilizer can comprise or consist of a non-ionic surfactant. The non-ionic surfactant can comprise or consist of polysorbates (e.g., poly sorbate 80, poly sorbate 20), alkylsaccharides alkyl ethers and alkyl glyceryl ethers, polyoxyethelene (4) lauryl ether; polyoxyethylene cetyl ethers, polyoxyethylene stearyl ethers, sorbitan fatty acid esters, polyoxyethylene fatty acid esters, or combinations thereof. The polypeptide can be formulated with a protein surfactant, such as recombinant human serum albumin as a stabilizer. Antioxidants or reducing agents can increase the stability of a polypeptide. The stabilizer can comprise or consist of an antioxidant or reducing agent. The reducing agent can comprise or consist of dithiothreitol, ethylenediaminetetraacetic acid, 2-Mercaptoethanol, Tris(2-carboxyethyl)phosphine hydrochloride, Tris(hydroxypropyl)phosphine, or combinations thereof. The antioxidant can comprise or consist of methionine, ascorbic acid, citric acid, alpha tocopherol, sodium bisulfite, ascorbyl palmitate, erythorbic acid, or combinations thereof. Chelating agents can stabilize polypeptides by reducing the activity of proteases. The stabilizer can comprise or consist of a chelating agent. The chelating agent can comprise or consist of ethylenediaminetetraacetic acid (EDTA), ethylene glycol-bis(R-aminoethyl ether)-N,N,N′,N′-tetraacetic acid (EGTA), metal complexes (e.g. Zn-protein complexes), or combinations thereof. Buffer agents can stabilize polypeptides by reducing the acid hydrolysis of polypeptides. The stabilizer can comprise or consist of a buffer agent. The buffer agent can comprise or consist of sucrose octa-sulfate, ammonium carbonate, ammonium phosphate, boric acid, sodium citrate, potassium citrate, lactic acid, 3-(N-morpholino)propanesulfonic acid (MOPS), 2-(N-morpholino)ethanesulfonic acid (MES), hydroxymethylaminomethane (Tris), calcium carbonate, calcium phosphate or combinations thereof.

The polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein also may be entrapped in or associated with microcapsules prepared, for example, by coacervation techniques or by interfacial polymerization (for example, hydroxymethylcellulose or gelatin-microcapsules and poly-(methylmethacylate) microcapsules, respectively), in colloidal delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein may be formulated or delivered with an anti-inflammatory agent. The anti-inflammatory agent can comprise or consist of a corticosteroid. The corticosteroid can comprise or consist of hydrocortisone, cortisone, ethamethasoneb (Celestone), prednisone (Prednisone Intensol), prednisolone (Orapred, Prelone), triamcinolone (Aristospan Intra-Articular, Aristospan Intralesional, Kenalog), methylprednisolone (Medrol, Depo-Medrol, Solu-Medrol), or dexamethasone (Dexamethasone Intensol). The anti-inflammatory can comprise or consist of a non-steroidal anti-inflammatory (NSAID). The NSAID can comprise or consist of aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

The polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein can be included in a pharmaceutical composition suitable for intravenous administration comprising one or more pharmaceutically acceptable excipients, carriers, and diluents. The polypeptides of the current disclosure can be administered suspended in a sterile solution. The solution can be one commonly used for administration of biological formulations, and comprises, for example, about 0.9% NaCl or about 5% dextrose. The solution can further comprise one or more of: buffers, for example, acetate, citrate, histidine, succinate, phosphate, potassium phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine, histidine, leucine, or arginine; antioxidants, for example, ascorbic acid, methionine; or chelating agents, for example, EDTA, or EGTA.

The polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein can be included in a pharmaceutical composition suitable for intramuscular or subcutaneous administration comprising one or more pharmaceutically acceptable excipients, carriers, and diluents. Formulations suitable for intramuscular or subcutaneous injection can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include ethanol, polyols (inositol, propyleneglycol, polyethylene-glycol, glycerol, cremophor and the like) and suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

The polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein can be formulated for topical administration as a cream, gel, paste, ointment, or emulsion. Excipients in a cream, gel, paste, ointment, or emulsion can comprise gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

The excipient used with the polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein will allow for storage, formulation, or administration of highly concentrated formulations. In certain embodiments, a highly concentrated fusion polypeptide(s) comprises at least about 1, 2, 3, 4, 5, 6, 7, 8, 9, 10, 15, 20, 25, 20, 25, 40, 45, 50 or more milligrams per milliliter.

The polypeptides and/or compositions of the current disclosure can be shipped/stored lyophilized and reconstituted before administration. Lyophilized ligand fusion polypeptide formulations can comprise a bulking agent such as, mannitol, sorbitol, sucrose, trehalose, and dextran 40. The lyophilized formulation can be contained in a vial comprised of glass. The fusion polypeptides when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

### Kits

Also described herein are kits comprising one or more of the polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein in a suitable container and one or more additional components selected from: instructions for use; a diluent, an excipient, a carrier, and a device for administration.

In an aspect, described herein is a method of preparing a soft tissue or muscle disease or disorder treatment comprising admixing one or more pharmaceutically acceptable excipients, carriers, or diluents and polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein. In an aspect, described herein is a method of preparing a soft tissue or muscle disease or disorder treatment for storage or shipping comprising lyophilizing one or more antibodies of the current disclosure.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1—Expression and Purification of Recombinant Proteins

Mammalian expression plasmids carrying genes with different tags were transiently transfected into CHO cells. The genes were expressed to produce proteins that were subsequently secreted into the culture medium. The proteins in the culture medium were visualized on polyacrylamide gels and their activities were measured by in vitro functional assays. Then the recombinant proteins in the culture medium were affinity purified. The purified proteins were visualized on polyacrylamide gels to evaluate the purity and assayed by in vitro functional assays to determine their biological activities.

Expression vector engineering: Mammalian expression vector pmax Cloning was used to make C-terminally 6×His-tagged, StrepII-tagged, and human IgG1 and IgG4 Fc-tagged vectors. The DNA fragments encoding the secreted myogenic factors were amplified by PCR from human open reading frame (ORF) clones, and subsequently inserted into the tagged vectors by In-Fusion cloning technology (Takara Bio Inc.).

Expressing secreted myogenic polypeptides: The expression vectors carrying the secreted myogenic factors were transiently transfected into ExpiCHO-S cells at a density of 6×10⁶ per ml by using ExpiFectamine CHO transfection kit (Thermo Scientific). After 18-22 hours, CHO feed and enhancer were added into the transfected culture. Then the expressed proteins were monitored by SDS-PAGE every 24 hours to achieve maximal expression level. In most of the cases, cell culture was collected at day 4, and cells were spun down. The supernatant was spun down again to get rid of cellular debris. The clarified culture supernatant containing the secreted myogenic factors was stored at −80° C. or immediately processed for use.

Measuring expression level of secreted myogenic polypeptides: To measure the improved expression level of the secreted myogenic factors, three protein analytical techniques were applied: sodium dodecyl sulfate polyacrylamide gel electrophoresis (SDS-PAGE), Western blotting, and enzyme-linked immunosorbent assay (ELISA). Western Blots were performed to identify the myogenic factors. ELISAs were used to measure the absolute amount of myogenic factors in the culture supernatant.

Isolation of engineered myogenic polypeptides: The expressed myogenic factors with different tags in the culture supernatants were affinity-purified by using different purification media. For Fc-fusion factors, either Protein A magnetic beads (GenScript) or Protein A membrane column (Takara Bio Inc.) were used to specifically bind to the Fc-fusion factors. For 6×His-tagged factors, NTA-magnetics beads (NEB) were used to isolate the factor.

### Example 2—Purified IGF2-hFcm Promoted Differentiation of Human Myoblast Cells

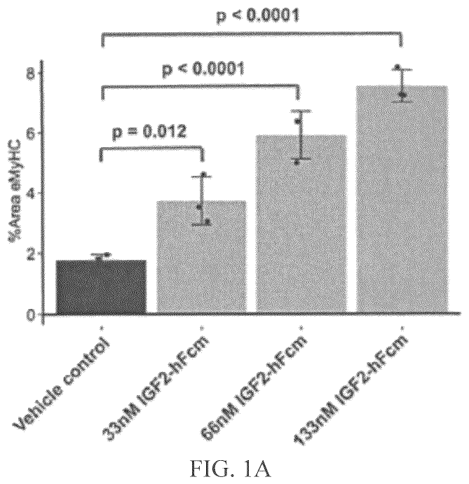

FIG. 1A: The suspension CHO cells were transiently transfected with the IGF2-hFcm encoding plasmid. IGF2-hFcm was affinity-purified by Protein A membrane column. The purified IGF2-hFcm was added into the culture of human myoblast cells for 96 hours. Myosin heavy chain (MyHC) was immunostained and imaged by a fluorescence microscope. The percentage area of MyHC of human myoblasts treated with the purified IGF2-hFcm is significantly higher than the percentage area of MyHC of human myoblasts treated with the vehicle control (One-Way ANOVA Tukey Honest Significant Difference, n=2-6).

| Condition | % MyHC | SD | p-value |
|---|---|---|---|
| Vehicle control | 1.787 | 0.186 | |
| 33 nM IGF2-hFcm | 3.734 | 0.790 | 0.012 |
| 66 nM IGF2-hFcm | 5.922 | 0.795 | 3.20E−05 |
| 133 nM IGF2-hFcm | 7.568 | 0.538 | 1.46E−06 |

### Example 3 IGF2-LhFc4 Promoted Differentiation of Human Myoblast Cells

The suspension CHO cells were transiently transfected with the IGF2-LhFc4 encoding plasmid. IGF2-LhFc4 was affinity-purified by Protein A membrane column. The purified IGF2-LhFc4 was added into the culture of human myoblast cells for 96 hours with daily media change. Myosin heavy chain (MyHC) was immunostained and imaged by a fluorescence microscope. The percentage area of MyHC of human myoblasts treated with the purified IGF2-LhFc4 is significantly higher than the percentage area of MyHC of human myoblasts treated with the vehicle control (One-Way ANOVA Tukey Honest Significant Difference, n=2-6).

| Condition | % MyHC | SD | p-value |
|---|---|---|---|
| Vehicle control | 1.384 | 0.285 | |
| hFc4L-IGF2 | 5.820 | 0.319 | 0.011 |
| IGF2-hFc4 | 6.901 | 0.537 | 0.004 |
| IGF2-LhFc4 | 6.237 | 1.848 | 0.007 |

### Example 4—Purified HSA-L-IGF2R61A Differentiation of Human Myoblast Cells

The suspension CHO cells were transiently transfected with the HSA-L-IGF2R61A encoding plasmid. HSA-L-IGF2R61A was affinity-purified by Protein A membrane column. The purified HSA-L-IGF2R61A was added into the culture of human myoblast cells for 96 hours with daily media change. Myosin heavy chain (MyHC) was immunostained and imaged by a fluorescence microscope. The percentage area of MyHC of human myoblasts treated with the purified HSA-L-IGF2R61A is significantly higher than the percentage area of MyHC of human myoblasts treated with the vehicle control (One-Way ANOVA Tukey Honest Significant Difference, n=2-6).

| Condition | % MyHC | SD | p-value |
|---|---|---|---|
| Vehicle control | 0.34 | 0.221 | |
| HSA-IGF2 | 7.079 | 2.009 | 0.013 |
| HSA-IGF2R61A | 6.914 | 2.691 | 0.014 |

### Example 5 IGF2 and IGF2 Receptors are Expressed in Human Myoblast

Bar graph and quantitation table of IGF2 and IGF2 receptor RNASeq expression in young (17-21 year old caucasian males) and aged human myoblast (68-69 year old caucasian males) cell lines. Myoblast were cultured growth media (GM) or 96 h in fusion media (FM). Fresh media was added every 24 h. Mean±SEM. n=6. Expression are expressed as FPKM. Significant p-values (Young GM˜Aged GM: 3.54E-04).

| n = 6 | IGF2 (FPKM) | SEM | p-val (n = 6) |
|---|---|---|---|
| Young GM | 13.11 | 3.275 | — |
| Aged GM | 3.413 | 1.12 | 3.54E−04 |
| Young FM | 17.68 | 6.42 | — |
| Aged FM | 13.08 | 3.67 | n.s. |
| n = 6 | IGF2R (FPKM) | SEM | P-val (n = 6) |
| Young GM | 74.93 | 9.45 | — |
| Aged GM | 75.01 | 6.89 | n.s. |
| Young FM | 82.35 | 3.43 | — |
| Aged FM | 88.44 | 9.86 | n.s. |

### Example 6 Sodium Butyrate Enhances Muscle Fusion

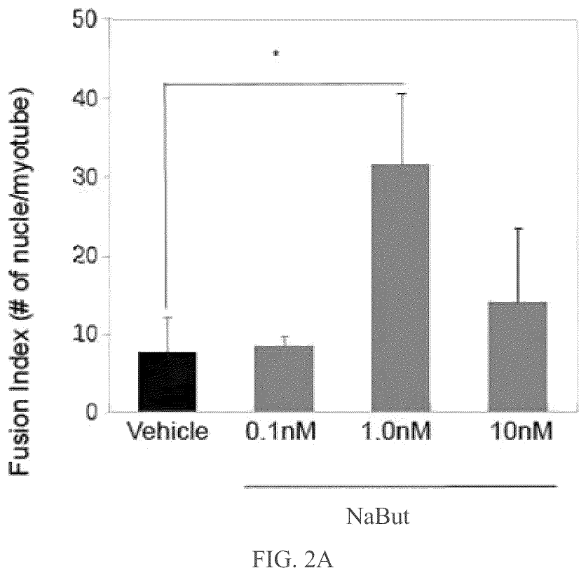

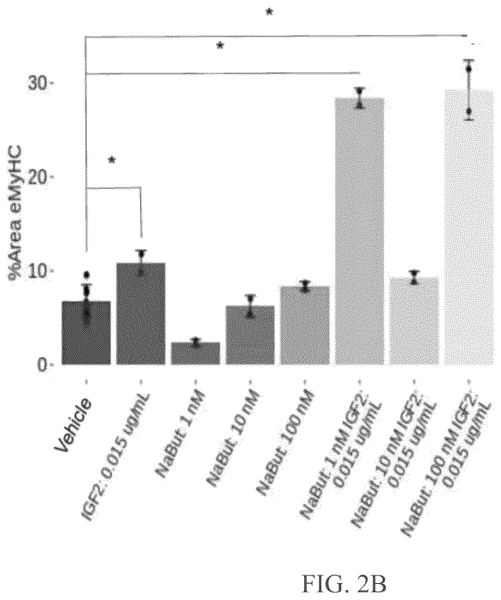

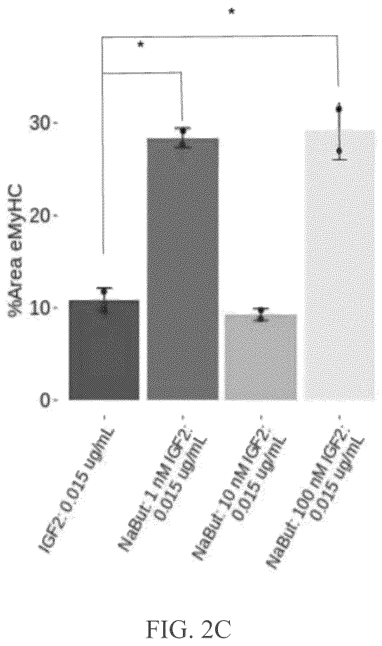

Mouse myoblasts were treated with PBS or sodium butyrate at concentrations 0.1 nM, 1 nM, and 10 nM. Myoblasts were cultured for 48 hours, with fresh media added every 24 hours. Cells were pulsed for 2-5 hours with EdU (30 uM), ethanol fixed, stained with Hoescht 3342, immunostained for proliferation—as measured by the percent of cells staining positive for EdU (% EdU)-, and immunostained for differentiation—as measured by the increase in cellular area staining positive for embryonic myosin heavy chain (% eMyHC) relative to the negative controls, which received media and vehicle only. When compared to untreated myoblasts, the cells treated with 1 nM of sodium butyrate had increased rates of fusion, as depicted in FIG. 2A. Significance was determined by a p-value less than 0.05 by the one-way ANOVA Tukey Honest Significant Difference test.

FIG. 2A: Bar graph of fusion index in response to sodium butyrate (NaBut) compared to vehicle. Myoblast were cultured 48 h in the presence of NaBut at indicated dose. Fresh media and NaBut were added every 24 h. Mean+̅S.D. Table quantitation of fusion index and p-values also shown. (*p<0.05 by Student's Two-tailed T-test, n=3-5)

| Condition | Fusion Index (nuclei/myotube) | p-value |
|---|---|---|
| vehicle | 7.56 | — |
| NaBut 0.1 nM | 8.44 | n.s. |
| NaBut 1 nM | 31.60 | 2.02E−3 |
| NaBut 10 nM | 14.00 | n.s. |

### Example 7 Sodium Butyrate Enhances IGF2 Activity

Human myoblast cells were treated with either PBS (vehicle), IGF2 (15 ng/mL), sodium butyrate, or IGF2 and sodium butyrate. Fresh media was added every 24 hours. After 96 hours, cells were pulsed for 2-5 hours with EdU (30 uM), ethanol fixed, stained with Hoescht 3342, immunostained for proliferation—as measured by the percent of cells staining positive for EdU (% EdU)-, and immunostained for differentiation—as measured by the increase in cellular area staining positive for embryonic myosin heavy chain (% eMyHC) relative to the negative controls, which received media and vehicle only. The total area of eMyHc positive cells was analyzed, and treated cells were compared to cells treated with the vehicle alone. Cells that had been treated with IGF alone and two conditions in which cells had been treated with IGF2 and sodium butyrate produced a significant increase in the amount of differentiation. There was a significant increase in the total area of eMyHC cells in the cells treated with 1 nM and 100 nM of sodium butyrate and IGF2, compared to the cells treated with IGF2 alone. Significance was determined by a p-value less than 0.05 by the one-way ANOVA Tukey Honest Significant Difference test.

FIG. 2B: Bar graph of fusion index of mouse myoblast in response to sodium butyrate (NaBut) compared to vehicle. Mouse myoblast were cultured 48 h in the presence of NaBut at indicated dose. Fresh media and NaBut were added every 24 h. Mean+̅S.D. Table quantitation of fusion index and p-values shown. (*p<0.05 by Student's Two-tailed T-test, n=3-5). Significant p-values (Vehicle˜IGF2: 0.015 ug/mL: 6.33E-06, Vehicle˜Naflut: 1 nM IGF2: 0.015 ug/mL: 1.79E-11, Vehicle˜-NaBut: 100 nM IGF2: 0.015 ug/mL: 1.79E-11)

Table of data for FIG. 2B

| Condition | % eMyHC | SD | p-value |
|---|---|---|---|
| Vehicle | 6.813 | 1.695 | — |
| IGF2: 0.015 ug/mL | 10.843 | 1.308 | |
| NaBut: 1 nM | 2.321 | 0.374 | |
| NaBut: 10 nM | 6.199 | 1.174 | |
| NaBut: 100 nM | 8.341 | 0.477 | |
| NaBut: 1 nM IGF2: 0.015 ug/mL | 28.387 | 1.036 | 1.79E−11 |
| NaBut: 10 nM IGF2: 0.015 ug/mL | 9.274 | 0.654 | |
| NaBut: 100 nM IGF2: 0.015 ug/mL | 29.239 | 3.185 | 1.79E−11 |

FIG. 2C Bar graph quantitation of % Area eMyHC+ human myoblast in response to indicated treatment compared to IGF2 (15 ng/mL). Myoblast were cultured 96 h in the presence of BMIP7 at indicated dose. Fresh media and BMP7 was added every 24 h. Mean+̅S.D. (*p<0.05 by One-Way Anova Tukey Honest Significant Difference, n=2-12)

Table of data for FIG. 2C

| Condition | % eMyHC | SD | p-value |
|---|---|---|---|
| IGF2: 0.015 ug/mL | 10.843 | 1.308 | |
| NaBut: 1 nM IGF2: 0.015 ug/mL | 28.387 | 1.036 | 6.18E−8 |
| NaBut: 10 nM IGF2: 0.015 ug/mL | 9.274 | 0.654 | |
| NaBut: 100 nM IGF2: 0.015 ug/mL | 29.239 | 3.185 | 3.50E−3 |

### Example 8 Sodium Butyrate Enhances IGF2 Activity

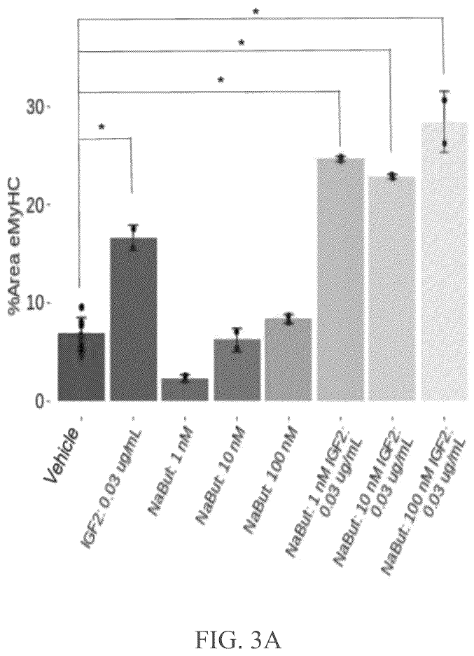

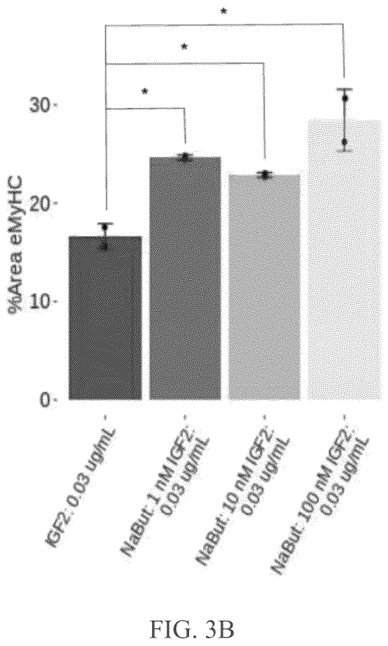

Human myoblast cells were treated with either PBS (vehicle), IGF2 (15 ng/mL), sodium butyrate, or IGF2 and sodium butyrate. Fresh media was added every 24 hours. After 48 hours, cells were pulsed for 2-5 hours with EdU (30 uM), ethanol fixed, stained with Hoescht 3342, immunostained for proliferation—as measured by the percent of cells staining positive for EdU (0% EdU)-, and immunostained for differentiation—as measured by the increase in cellular area staining positive for embryonic myosin heavy chain (0% eMyHC) relative to the negative controls, which received media and vehicle only. The total area of eMyHc positive cells was analyzed, and treated cells were compared to cells treated with the vehicle alone, as seen in FIG. 3A. Myoblasts that had been treated with either 0.03 ug/mL of IGF2 or with IGF2 in combination with sodium butyrate showed a significant increase in the eMyHC+ area when compared to cells cultured with the vehicle alone.

FIG. 3A: Bar graph of % Area eMyHC+ age human myoblast (68 year old caucasian male) in response to indicated treatment compared to Vehicle (vehicle). Myoblast were cultured 96 h in the presence of factors at indicated dose. Fresh media and factors were added every 24 h. Mean+̅S.D. Significant p-values (Vehicle˜IGF2: 0.03 ug/mL: 1.42E-08, Vehicle˜NaBut: 1 nM IGF2: 0.03 ug/mL: 1.79E-11, Vehicle˜NaBut: 10 nM IGF2: 0.03 ug/mL: 1.80E-11, Vehicle˜NaBut: 100 nM IGF2: 0.03 ug/mL: 1.79E-11). FIG. 3B Bar graph quantitation of % Area eMyHC+ human myoblast (68 year old caucasian male) in response to indicated treatment compared to IGF2 (15 ng/mL). Myoblast were cultured 96 h in the presence of BMP7 at indicated dose. Mean+̅S.D. Significant p-values (IGF2˜NaBut: 1 nM IGF2: 0.03 ug/mL: 1.88E-3, Vehicle˜NaBut: 10 nM IGF2: 0.03 ug/mL: 4.80E-3, Vehicle˜NaBut: 100 nM IGF2: 0.03 ug/mL: 1.87E-3) (*p<0.05 by One-Way ANOVA Tukey Honest Significant Difference, n=2-12) Table of data for FIG. 3A

| Condition | % cMyHC | SD | p-value |
|---|---|---|---|
| Vehicle | 6.813 | 1.695 | — |
| IGF2: 0.03 ug/mL | 16.620 | 1.301 | 1.42E−08 |
| NaBut: 1 nM | 2.321 | 0.374 | |
| NaBut: 10 nM | 6.199 | 1.174 | |
| NaBut: 100 nM | 8.341 | 0.477 | |
| NaBut: 1 nM IGF2: 0.03 ug/mL | 24.615 | 0.258 | 1.79E−11 |
| NaBut: 10 nM IGF2: 0.03 ug/mL | 22.821 | 0.234 | 1.80E−11 |
| NaBut: 100 nM IGF2: 0.03 | 28.427 | 3.136 | 1.79E−11 |
| ug/mL | | | |

The myoblasts that had been treated with a combination of IGF2 and sodium butyrate were compared to the cells treated with IGF2 alone. There was a significant increase in all cells treated with the combination compared to cells treated with IGF2 alone, as depicted in FIG. 3B and Table 12. Significance was determined by a p-value less than 0.05 by the one-way ANOVA Tukey Honest Significant Difference test.

Table of data for FIG. 3B

| Condition | % cMyHC | SD | p-value |
|---|---|---|---|
| IGF2: 0.03 ug/mL | 16.620 | 1.301 | — |
| NaBut: 1 nM IGF2: 0.03 ug/mL | 24.615 | 0.258 | 1.88E−3 |
| NaBut: 10 nM IGF2: 0.03 ug/mL | 22.821 | 0.234 | 4.80E−3 |
| NaBut: 100 nM IGF2: 0.03 | 28.427 | 3.136 | 1.87E−3 |
| ug/mL | | | |

### Example 9 IGF2 Enhances MYOG Expression in DM1 Human Myoblast Cells

Bar graph of myogenic gene expression fold change in DM1 human myoblast in response to indicated treatment compared to FM (vehicle). Myoblasts were cultured 48h in the presence of factors (BMIP7 50 ng/mL, Butyrate 100 nM, IGF2 200 ng/mL). Mean+̅S.D. Significant p-values (FM IGF2: 4.94E-04, FM˜IGF2_Naflut: 6.53E-03) (*p<0.01) Table of mean and p-value of MYF5, MYOD1, and MYOG (n=3).

Table of data

| Con- | | MYF5 | | MYOD1 | | MYOG |
|---|---|---|---|---|---|---|
| dition | MYF5 | p-value | MYOD1 | p-value | MYOG | p-value |
| FM | 1.000 | | 1.000 | | 1.000 | |
| BMP7 | 0.709 | n.s. | 0.709 | n.s. | 0.361 | n.s. |
| NaBut | 1.128 | n.s. | 1.095 | n.s. | 1.020 | n.s. |
| IGF2 | 0.730 | n.s. | 1.252 | n.s. | 2.972 | 4.94E−04 |
| IGF2 | 0.820 | n.s. | 1.500 | n.s. | 3.483 | 6.53E−03 |
| NaBut | | | | | | |

### Example 10 IGF2 Receptor is Expressed on Chondrocyte and Osteocytes

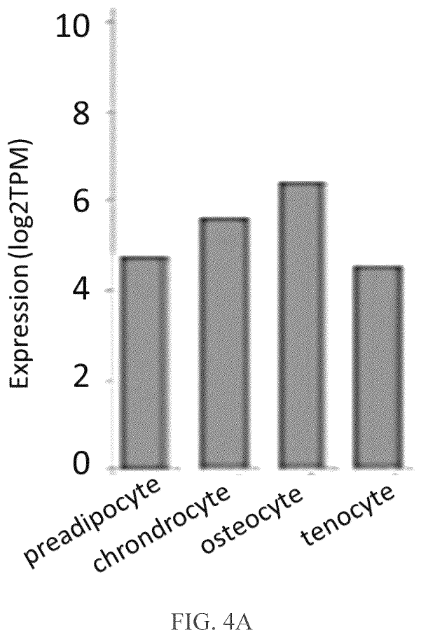

FIG. 4A: Bar graph showing IGF2 receptors are expressed on cartilage-associated cells. Data is derived from Ramilowsky et al., Nature 2015.

Table of data for FIG. 4A RNA Expression (TPM)

| Cell Type | IGF2R |
|---|---|
| Preadipocyte (Subcutaneous) | 27.083 |
| Chondrocyte | 47.63 |
| Osteocyte | 83.96 |
| Tenocyte | 23.12 |

### Example 11 IGF2 Treatment Promotes Proliferation and Fusion in DM1 Human Myoblast (32 Year Old Caucasian Female) Cells

Bar graph of % EdU+ human myoblast (32 year old caucasian female) and % area MyHC in response to IGF2. Myoblast were cultured 72 h for proliferation and 96 h for fusion in the presence of indicated factor. MeanRS.D. MeanRSD. Significant p-values (EdU: Vehicle˜IGF2: 6.8E-3, % eMyHC Area: Vehicle˜IGF2: 1.9E-4) (*p<0.05 by Students Two-Tailed T-test, n=3-6).

Table of data

| n = 3-6 | EdU FC | s.d. | p-val |
|---|---|---|---|
| Vehicle | 1.0 | 0.02 | |
| IGF2 | 2.18 | 0.32 | 6.8E−3 |

Table of data

| n = 3 | % eMyHC are | s.d. | p-val |
|---|---|---|---|
| Vehicle | 0.45 | 0.02 | |
| IGF2 | 5.49 | 0.54 | 1.9E−4 |

### Example 12 IGF2 Enhances MYH3, CKM, and ATP1B1 Expression in DM1 Human Myoblast (32 Year Old Caucasian Female) Cells

Bar graph of MYH3 and CKM expression fold change in DM1 human myoblast (32 year old caucasian female) in response to indicated treatment compared to vehicle. Myoblasts were cultured 96 h in the presence of factors (IGF2 200 ng/mL). MeanRS.D. Significant p-values (MYH3: Vehicle IGF2: 1.13E-03, CKM: Vehicle˜IGF2: 7.67E-03) Bar graph of ATP1B1 expression fold change in DM1 human myoblast (32 year old caucasian female) in response to indicated treatment compared to FM (vehicle). Myoblasts were cultured 48 h in the presence of factors (IGF2 200 ng/mL). MeanRS.D. Significant p-values (Vehicle˜IGF2: 3.11E-05) (*p<0.05 by Students Two-Tailed T-test, n=3).

Table of data

| | MYH3 | p-val | CKM | p-val |
|---|---|---|---|---|
| Vehicle | 1 | | 1 | |
| IGF2 | 14.833 | 1.13E−03 | 5.165 | 7.67E−03 |

Table of data

| n = 3 | ATP1B1 | p-val |
|---|---|---|
| Vehicle | 1 | |
| IGF2 | 3.01789 | 3.11E−05 |

### Example 13 Systemic Administration of IGF2/NaB Protects Against Aging Induced Muscle Dysfunction

Subcutaneous injection of IGF2(50 ug/kg) or NaB (1.2 g/kg), IGF2/NaB (150 ug/kg; 1.2 g/kg) or vehicle (PBS) were administered to 21-24M old mice for 14 days. Muscle function was assessed at days 13 and 14. Grip strength force assessed at day 13. The first graph shows Bothlimb grip strength force, ****p<0.0001, **p=0.0043, **p=0.001 (One-way ANOVA, multiple comparisons). Forelimb force, ****p<0.0001, *p=0.0368, *p=0.0187 (One-way ANOVA, multiple comparisons). Treadmill performance measured at day 14 using an induced treadmill running model set to progressively increase speed 2 m/min every subsequent 2 min. Distance ran shown. ***p=0.0005, *p=0.0459, ****p<0.0001 (One-way ANOVA, multiple comparisons) Time to exhaustion ***p=0.0002, **p=0.0024 (One-way ANOVA, multiple comparisons) Maximum speed ***p=0.0004, **p=0.0013 Work in kj**p=0.0026, **p=0.0035 (One-way ANOVA, multiple comparisons).

### Example 14 Systemic Administration of IGF2/NaB is Safe

Subcutaneous injection of vehicle or IGF2/NaB were administered to 21M old mice for 14 days, blood and serum were collected to assess complete blood count and a metabolic panel for liver, kidney and pancreas function. 4 representative graphs out of 37 readouts measured showing the white blood cell count (Unpaired t-test, p=0.8020), Albumin concentration (Unpaired t-test, p>0.9999), Creatinine concentration (Unpaired t-test, p=0.5490) and Calcium concentration (Unpaired t-test, p=0.811).

### Example 15 Systemic Administration of IGF2/but Protects Against Dexamethasone Induced Muscle Atrophy

Dexamethasone (25 mg/kg i.p.) was administered to 12 weeks old mice for 14 days simultaneously with a subcutaneous injection of IGF2/NaB (150 ug/kg; 1.2 g/kg) or vehicle (PBS). Muscle function was assessed at day 13-14. Grip strength force assessed at day 13, graphs showing bothlimb force and specific bothlimb force measured on Day 13. Specific bothlimb force calculated as the ratio of bothlimb force in mN over the weight in g, ***p=0.0003, ***p=0.0004 (Unpaired t-test). Grip strength force assessed at day 13, graphs showing forelimb force and forelimb specific force measured on Day 13. Specific forelimb force calculated as the ratio of forelimb force in mN over the weight in g, **p=0.0012, ***p=0.0005 (Unpaired t-test). At day 15, mice were euthanized and TAs were collected for histological analysis, graphs showing muscle fiber size distribution assessed using SMASH software. **p=0.054, *p=0.037, and ****p<0.0001 (2-way ANOVA, multiple comparisons).

### Example 16 BMP7 Induces Myoblast Proliferation

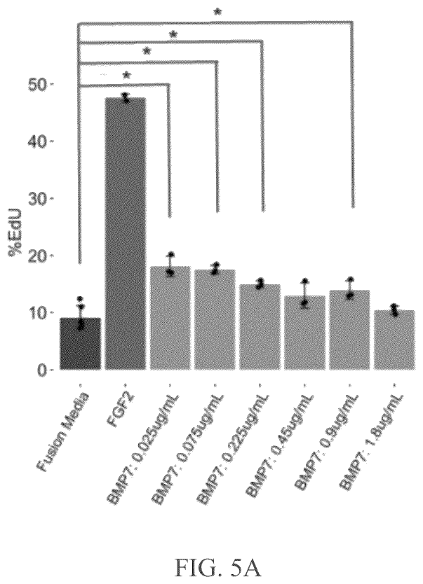

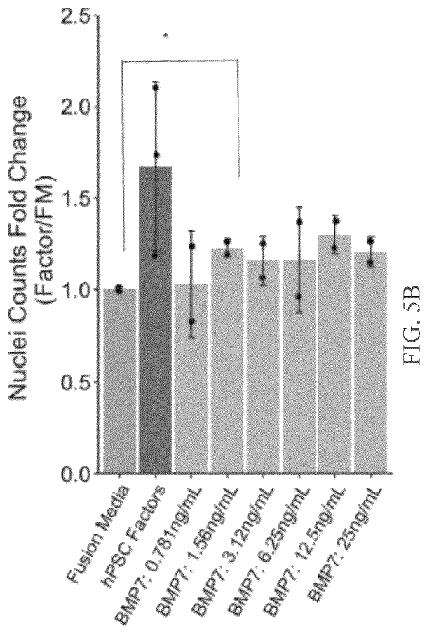

FIG. 5A) Bar graph quantitation of % EdU+ mouse myoblast in response to BMP7. Myoblast were cultured 48 h in the presence of BMP7 at indicated dose. Fresh media and BMP7 was added every 24 h, followed by 2 hour EdU pulse and fixation. Mean+̅S.D. Significant p-values (Vehicle BMP7 0.025 ug/mL: 1.21E-07, Vehicle˜BMP7 0.075 ug/mL: 8.05E-07, Vehicle˜BMP7 0.2255 ug/mL: 3.37E-03, Vehicle˜BMP7 0.9 ug/mL: 4.99E-02). FIG. 5B) Bar graph quantitation of % EdU+ human myoblast (68 year old caucasian male) in response to BMP7. Myoblast were cultured 72 h in the presence of BMP7 at indicated dose. Cells were pulsed with EdU for 4 hours before fixation. Mean+̅S.D. Significant p-values (Vehicle˜BMP7 1.56 ng/mL: 0.04). (*p<0.05 by Welch's One-Tailed T-test, n=2)

Table of data for FIG. 5A - BMP7 Mouse Myoblast Proliferation

| BMP7 | % EdU | p-value |
|---|---|---|
| Vehicle | 10.56 | — |

| | | | |
|---|---|---|---|
| 0.025 | ug/mL | 18.08 | 1.21E−07 |
| 0.075 | ug/mL | 17.53 | 8.05E−07 |
| 0.2255 | ug/mL | 14.93 | 3.37E−03 |
| 0.45 | ug/mL | 12.93 | n.s. |
| 0.9 | ug/mL | 13.91 | 4.99E−02 |
| 1.8 | ug/mL | 10.37 | n.s. |

Table of data for FIG. 5B -BMP7 Human Myoblast Proliferation

| Human Myoblast | Nuclei Counts | p-value |
|---|---|---|
| Vehicle | 2083.5 | — |

| | | | |
|---|---|---|---|
| 0.78 | ng/mL | 2144.5 | n.s. |
| 1.56 | ng/mL | 2552.5 | 0.04 |
| 3.12 | ng/mL | 2408 | n.s. |
| 6.25 | ng/mL | 2422.5 | n.s. |
| 12.5 | ng/mL | 2706 | n.s. |
| 25 | ng/mL | 2509 | n.s. |

### Example 17 Leucine Enhances BMP7 Mitogenic Activity

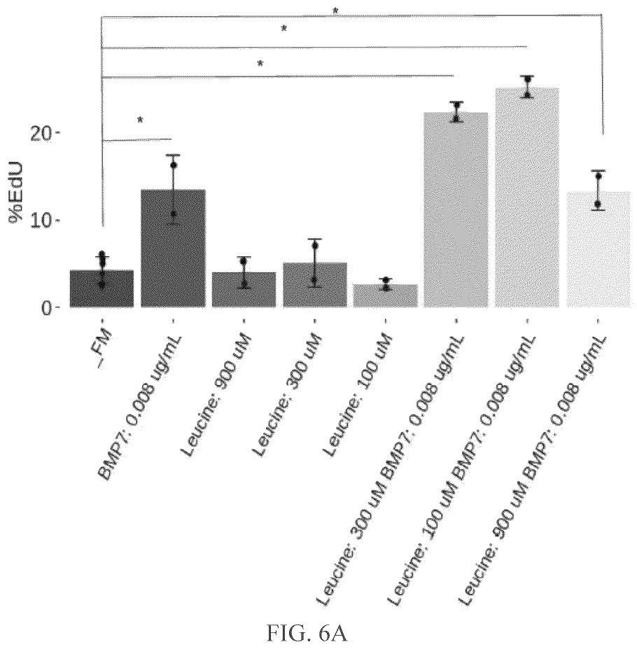

FIG. 6A) Bar graph of % EdU+ mouse myoblast cells compared to vehicle. Mouse myoblast were cultured 48 h in the presence of indicated factors, followed by a 2 hour EdU pulse prior to fixation. Mean+̅S.D. Significant p-values (FM˜BMP7: 0.008 ug/mL: 3.17E-02, FM˜Leucine: 300 uM BMP7: 0.008 ug/mL: 2.77E-03, FM˜Leucine: 100 uM BMP7: 0.008 ug/mL: 3.72E-05, FM˜Leucine: 900 uM BMP7: 0.008 ug/mL: 2.52E-03). (*p<0.05 by One-Way ANOVA Tukey Honest Significant Difference, n=2-6)

### Example 18 Hyaluronic Acid (HA) Enhances BMP7 Mitogenic Activity

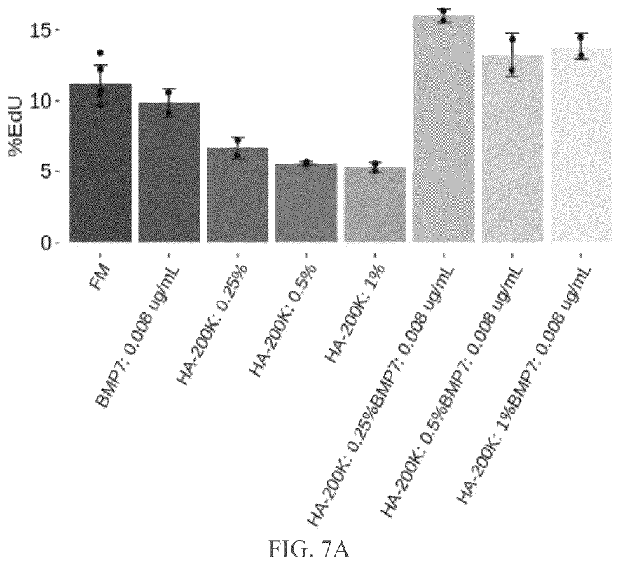

FIG. 7A: Bar graph of % EdU+ mouse myoblast cells compared to vehicle. Mouse myoblast were cultured 48 h in the presence of indicated factors. EdU pulse and fixation. Mean+̅S.D. (*p<0.05 by Student's One-Tailed T-Test of increased activity, n=2-6)

### Example 19 BMP7 Receptors are Expressed in Human Myoblast

FIG. 8A: Bar graph of BMP7 receptor RNASeq expression in young and aged human myoblast (68-69 year old caucasian males) cell lines. Myoblast were cultured 96 h in fusion media. Fresh media was added every 24 h, followed by RNA extraction and sequencing. Mean+̅SEM. n=3. Expression are expressed as FPKM.

### Example 20 Treatment for Chondrocyte Proliferation in Cartilage Injury and Osteoarthritis

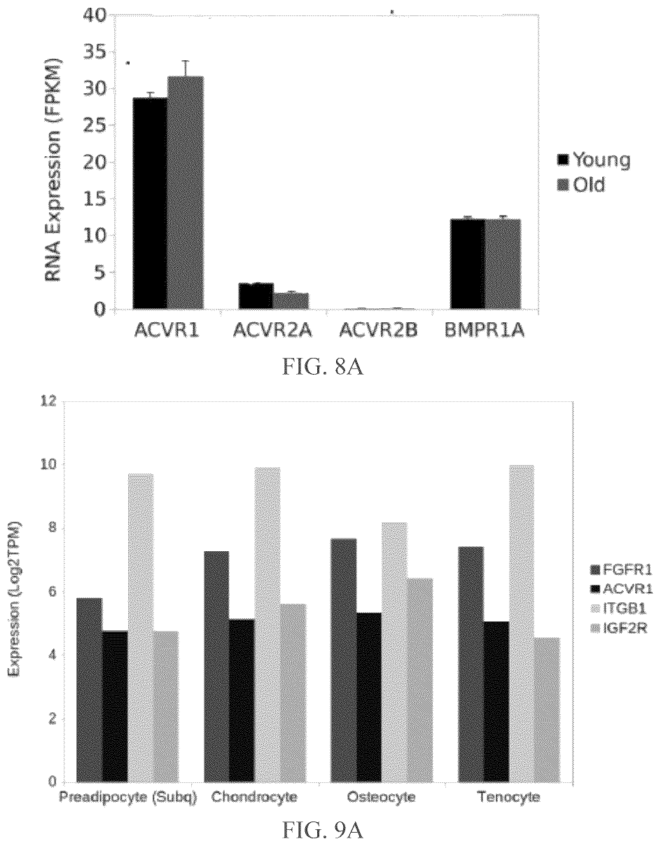

FIG. 9A: Bar graph showing BMP7 receptors are expressed on cartilage-associated cells. Data derived from Ramilowsky et al., Nature, 2015.

### Example 21—FGF17-hFcm Promotes Proliferation of Mouse Myoblasts

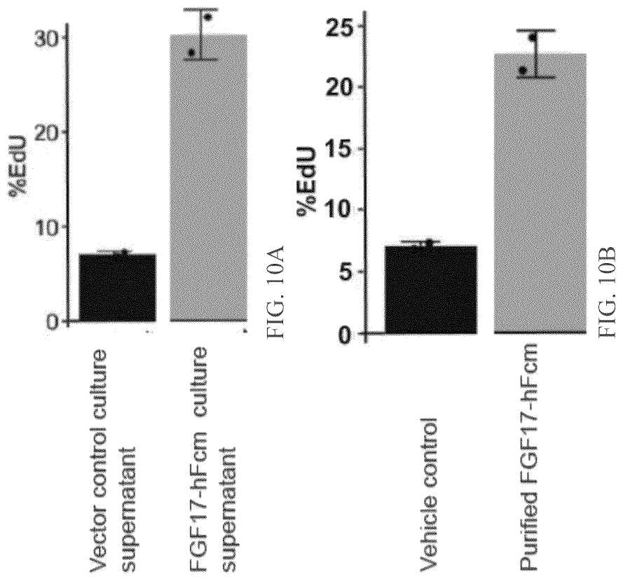

FIG. 10A) The suspension CHO cells were transiently transfected with either the empty control plasmid or the FGF17-hFcm encoding plasmid. After four days, the culture supernatants were collected and added into the culture of mouse myoblast cells for 48 hours, followed by a 2 hour EdU pulse prior to fixation. The percentage of EdU+ mouse myoblasts treated with the culture supernatant of CHO cells expressing FGF17-hFcm is significantly higher

than the percentage of EdU+ mouse myoblasts treated with either vehicle control or the culture supernatant of CHO cells expressing the empty control vector (One-Way ANOVA Tukey Honest Significant Difference, n=2-6). FIG. 10B) The suspension CHO cells were transiently transfected with the FGF17-hFcm encoding plasmid. After four days, the culture supernatants were collected and FGF17-hFcm was affinity-purified by Protein A membrane column. The purified FGF17-hFcm was added into the culture of mouse myoblast cells for 48 hours, followed by a 2 hour EdU pulse and fixation. The percentage of EdU+ mouse myoblasts treated with the purified FGF17-hFcm is significantly higher than the percentage of EdU+ mouse myoblasts treated with the culture supernatant of CHO cells expressing the empty control vector (One-Way ANOVA Tukey Honest Significant Difference, n=2-6).

Table of data for FIG. 10A

| Sample | % EdU | SD | p_value |
|---|---|---|---|
| Vector control culture supernatant | 7.069 | 0.354 | |
| FGF17-hFcm culture supernatant | 30.285 | 2.650 | 8.88E−6 |

Table of data for FIG. 10B

| Sample | % EdU | SD | p_value |
|---|---|---|---|
| Vehicle control | 4.884 | 1.080 | |
| Purified FGF17-hFcm | 22.761 | 1.880 | 2.04E−5 |

### Example 22: FGF17 Mutants Improved Protein Expression Levels in CHO Cells

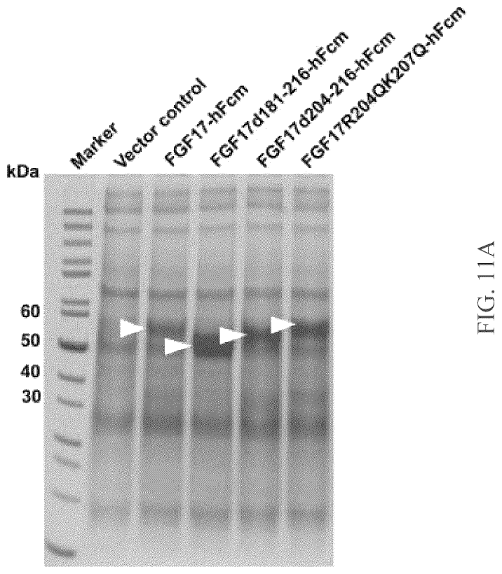

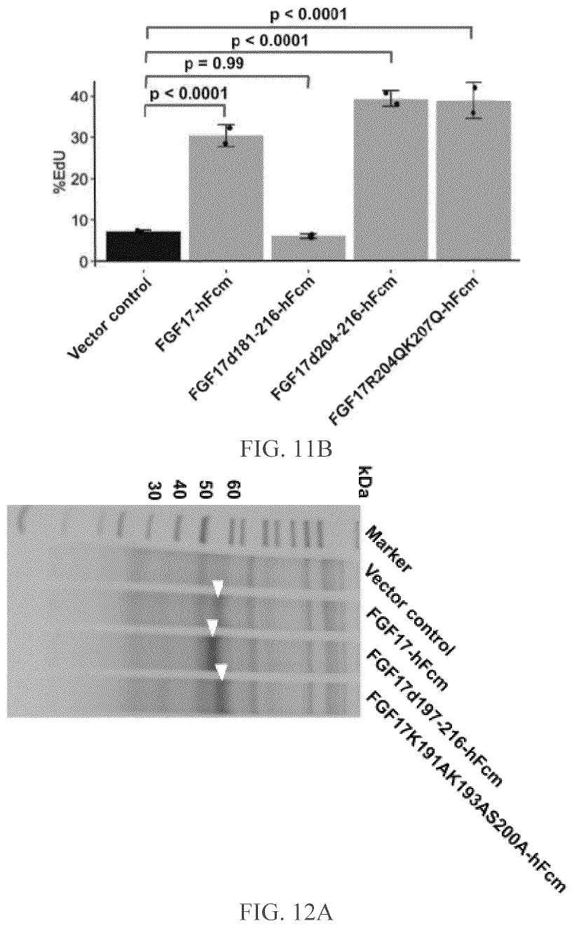

FIG. 11A: SDS-PAGE of culture supernatants from CHO cells transiently transfected with FGF17-hFcm or its mutants encoding plasmids (FGF17d181-216-hFcm, FGF17d204-216-hFcm, FGF17R204QK207Q-hFcm). The expressed FGF17-hFcm proteins and the mutant proteins were indicated by the white arrowheads. FIG. 11B) The culture supernatants from CHO cells transiently transfected with different FGF17 encoding plasmids were added into the culture of mouse myoblast cells for 48 hours followed by 2 hour EdU pulse and fixation. The percentage of EdU+ mouse myoblasts treated with the culture supernatants of CHO cells expressing wild type FGF17-hFcm or mutants FGF17-hFcm (AA204-216 deletion mutant and R204QK207Q point mutation mutant) is significantly higher than the percentage of EdU+ mouse myoblasts treated with the culture supernatant of CHO cells expressing the empty control vector (One-Way ANOVA Tukey Honest Significant Difference, n=2-6).

### FIG. 11B

| Sample | % EdU | SD | p_value |
|---|---|---|---|
| Vector control | 7.069 | 0.354 | |
| FGF17-hFcm | 30.285 | 2.650 | 1.08E−4 |
| FGF17d181-216-hFcm | 6.000 | 0.533 | 0.99 |
| FGF17d204-216-hFcm | 39.298 | 2.002 | 1.22E−5 |
| FGF17R204QK207Q-hFcm | 38.831 | 4.343 | 1.35E−5 |

### Example 23 FGF17 Mutants Improved Protein Expression Levels in CHO Cells

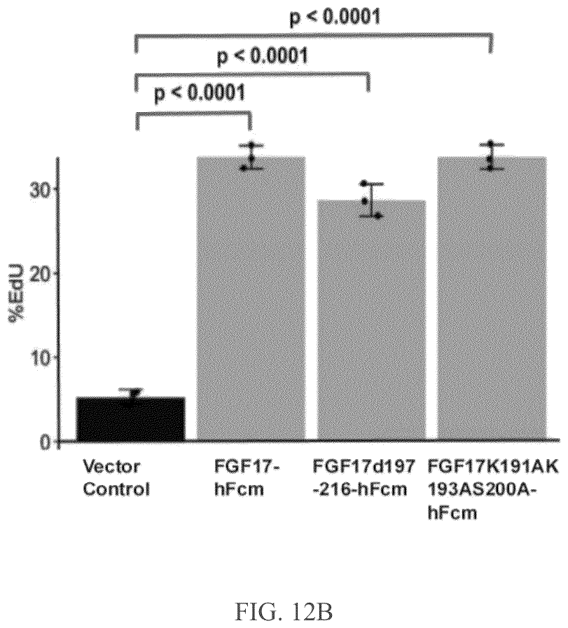

FIG. 12A) SDS-PAGE of culture supernatants from CHO cells transiently transfected with FGF17-hFcm or its mutants encoding plasmids (FGF17d197-216-hFcm, FGF17K191AK193AS200A-hFcm). The expressed FGF17-hFcm proteins and the mutant proteins were indicated by the white arrowheads. FIG. 12B) The culture supernatants from CHO cells transiently transfected with different FGF17 encoding plasmids were added into the culture of mouse myoblast cells for 48 hours, followed by 2 hour EdU pulse and fixation. The percentage of EdU+ mouse myoblasts treated with the culture supernatants of CHO cells expressing wild type FGF17-hFcm or mutants FGF17-hFcm (AA197-216 deletion mutant and K191AK193AS200A point mutation mutant) is significantly higher than mouse myoblasts treated with the culture supernatant of CHO cells expressing the empty control vector (One-Way ANOVA Tukey Honest Significant Difference, n=2-6).

### FIG. 12B

| Sample | % EdU | SD | p_value |
|---|---|---|---|
| Vector control | 5.288 | 0.883 | |
| FGF17-hFcm | 33.806 | 1.351 | 5.46E−10 |
| FGF17d197-216-hFcm | 28.699 | 1.926 | 5.30E−9 |
| FGF17K191AK193AS200A-hFcm | 33.828 | 1.434 | 5.42E−10 |

### Example 24 Human Serum Albumin (HSA) Fusion FGF17 is More Stable in Culture Medium Than FGF17 without HSA Fusion Tag

HSA-FGF17 and FGF17 were incubated in culture medium at 37 deg CO2 incubator. At different time point (day 0, 1, 3, 5, and 7), an aliquot was taken and stored at −80 deg. The activity of each sample was evaluated by mouse myoblast in vitro proliferation assay. The nuclei count at each time point from proliferation assay was normalized to day 0 nuclei count.

### Example 25 Differential Induction of Myogenic Gene Expression by FGF17 in Mouse Myoblasts

Myogenic gene RNA expression (fold change to FM) in response to vehicle (FM) in mouse myoblast cell lines monitored by real-time qPCR. Myoblast were cultured 48 h in fusion media. Mean+̅SD. n=3. (Y,Z) Differential induction of myogenic gene expression by FGF17 in human myoblast myoblasts. Quantitation table of myogenic gene RNA expression (fold change to FM-fusion media (DMEM+200 horse serum)) in response to FM or rh-FGF17 in aged human myoblast cell lines by real-time qPCR. Myoblast were cultured (B) 48 hours or (C) 72 hours in FM. Mean+̅SD. n=3.

| Condition | Mean | SD | p-value |
|---|---|---|---|
| Gene: Pax7 | | | |
| FM | 1.011 | 0.185 | — |
| FGF17 | 6.118 | 0.920 | 7.05E−04 |
| Gene: Myf5 | | | |
| FM | 1.002 | 0.071 | — |
| FGF17 | 1.376 | 0.201 | 1.69E−02 |
| Gene: Myod1 | | | |
| FM | 1.002 | 0.086 | — |
| FGF17 | 1.230 | 0.158 | n.s. |
| 48 hr | MYF5 | | |
| FM | 1.04 | 0.354 | |
| FGF17 | 1.922 | 0.234 | 6.06E−03 |
| 48 hr | MYOD1 | | |
| FM | 1.001 | 0.059 | |
| FGF17 | 0.604 | 0.095 | 2.76E−03 |
| 48 hr | MYOG | | |
| FM | 1.013 | 0.193 | |
| FGF17 | 2.353 | 0.016 | 5.77E−03 |
| 72 hr | MYF5 | | |
| FM | 1.023 | 0.273 | |
| FGF17 | 0.499 | 0.234 | 1.84E−03 |
| 72 hr | MYOD1 | | |
| FM | 1.055 | 0.423 | |
| FGF17 | 1.627 | 0.094 | n.s. |
| 72 hr | MYOG | | |
| FM | 1.092 | 0.542 | |
| FGF17 | 13.124 | 0.015 | 6.38E−04 |

### Example 26 FGF17 Receptor is Expressed in Human Myoblasts

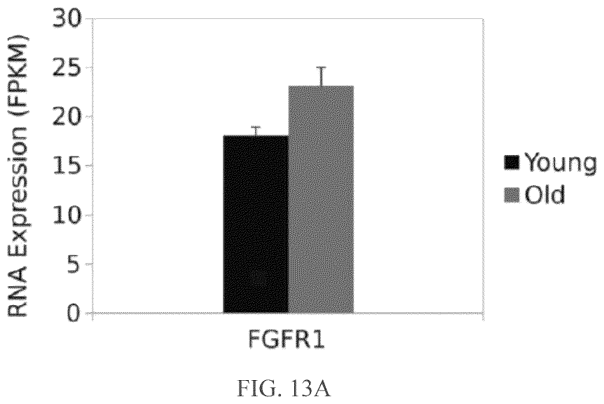

FIG. 13A: Bar graph of FGF17 receptor RNASeq expression in young and aged human myoblast cell lines. Myoblast were cultured 96 h in fusion media with fresh media added every 24 h, followed by RNA extraction and sequencing. Mean+̅SEM. n=3. Expression values are reported as FPKM.

| GeneName | Young (n = 6) | Old (n = 6) | Young_SEM | Old_SEM |
|---|---|---|---|---|
| FGFR1 | 18.069 | 23.122 | 0.843 | 1.836 |

### Example 27 Heparin Enhances FGF17 Mitogenic Activity

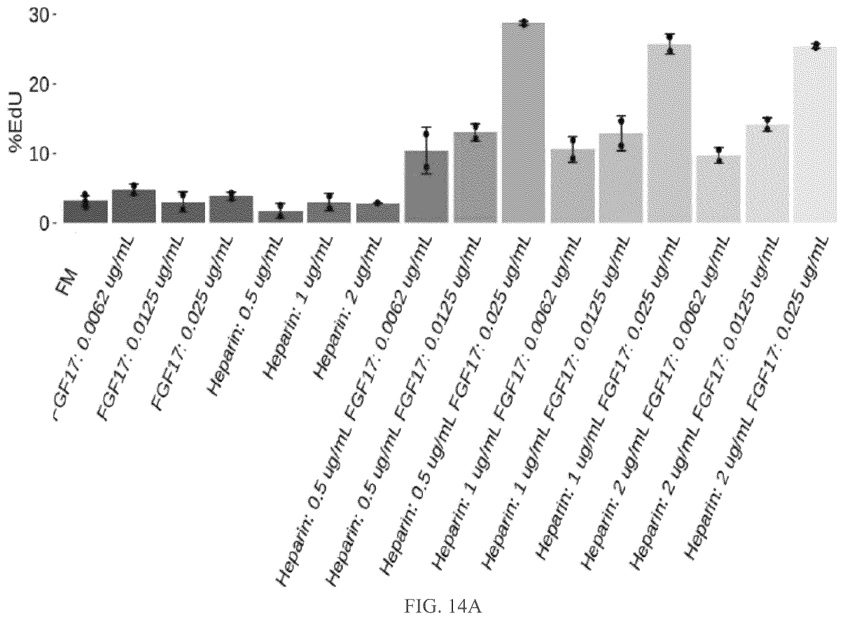

FIG. 14A: Bar graph of % EdU+ mouse myoblast cells compared to vehicle. Myoblast were cultured 48 h in the presence of indicated factors. Fresh media and factors were added every 24 h, followed by 2 hour EdU pulse and fixation. Mean+̅S.D. Table quantitation of % EdU and p-values (*p<0.05 by One-Way ANOVA Tukey Honest Significant Difference, n=2-6)

| | % | | |
|---|---|---|---|
| Condition | EdU | SD | p-value |
| Vehicle | 3.14 | 0.76 | — |
| FGF17: 0.0062 ug/mL | 4.81 | 0.79 | n.s. |
| FGF17: 0.0125 ug/mL | 3.02 | 1.46 | n.s. |
| FGF17: 0.025 ug/mL | 3.85 | 0.59 | n.s. |
| Heparin: 0.5 ug/mL | 1.77 | 1.06 | n.s. |
| Heparin: 1 ug/mL | 3.01 | 1.27 | n.s. |
| Heparin: 2 ug/mL | 2.85 | 0.02 | n.s. |
| Heparin: 0.5 ug/mL FGF17: 0.0062 ug/mL | 10.42 | 3.36 | 9.19E−04 |
| Heparin: 0.5 ug/mL FGF17: 0.0125 ug/mL | 13.01 | 1.24 | 1.09E−06 |
| Heparin: 0.5 ug/mL FGF17: 0.025 ug/mL | 28.73 | 0.29 | p < 0.4E−22 |
| Heparin: 1 ug/mL FGF17: 0.0062 ug/mL | 10.58 | 1.86 | 6.07E−04 |
| Heparin: 1 ug/mL FGF17: 0.0125 ug/mL | 12.88 | 2.50 | 1.54E−06 |
| Heparin: 1 ug/mL FGF17: 0.025 ug/mL | 25.75 | 1.45 | p < 0.4E−22 |
| Heparin: 2 ug/mL FGF17: 0.0062 ug/mL | 9.74 | 1.13 | 4.73E−03 |
| Heparin: 2 ug/mL FGF17: 0.0125 ug/mL | 14.16 | 0.96 | 5.38E−08 |
| Heparin: 2 ug/mL FGF17: 0.025 ug/mL | 25.44 | 0.34 | p < 0.4E−22 |

### Example 28 Hyaluronic Acid (HA) Enhances FGF17 Mitogenic Activity

FIG. 15A: Bar graph of % EdU+ mouse myoblast cells compared to vehicle. Myoblast were cultured 48 h in the presence of indicated factors. Fresh media and factors were added every 24 h, followed by 2 hour EdU pulse and fixation. Mean+̅S.D. Table quantitation of % EdU and p-values shown. (<p0.05 by Student's One-Tailed T-Test of increased activity, n=−2-6)

| Condition | N | % Edu | SD | p-value |
|---|---|---|---|---|
| FM | 6 | 11.162 | 1.386 | — |
| FGF17: 0.025 ug/mL | 2 | 17.870 | 2.827 | n.s. |
| HA-200K: 0.25% | 2 | 6.672 | 0.773 | n.s. |
| HA-200K: 0.5% | 2 | 5.561 | 0.116 | n.s. |
| HA-200K: 1% | 2 | 5.276 | 0.366 | n.s. |
| HA-200K: 0.25% FGF17: 0.025 ug/mL | 2 | 26.970 | 9.174 | 2.10E−02 |
| HA-200K: 0.5% FGF17: 0.025 ug/mL | 2 | 27.446 | 6.768 | 7.53E−03 |
| HA-200K: 1% FGF17: 0.025 ug/mL | 2 | 28.252 | 2.511 | 2.49E−04 |

### Example 29—Dextran Sulfate (DS) Enhances FGF17 Mitogenic Activity

Bar graph of total EdU+ mouse myoblast cells compared per condition. Myoblast were cultured 48 h in the presence of indicated factors. Fresh media and factors were added every 24 h, followed by 2 hour EdU pulse and fixation. Mean+̅Standard Deviation(SD) Table quantitation of EdU counts per field and p-values for mouse assay (*p<0.05 by Student's One-Tailed T-Test of increased activity, n=3-6, Synergy values <1 are evidence of effect). Bar graph of total EdU+ human myoblast cells per condition. Myoblast were cultured 72 h in the presence of indicated factors, followed by 4 hour EdU pulse and fixation. Fresh media and factors were added every 24 h. Mean+̅Standard Deviation (SD). Table quantitation of +EdU counts and p-values for human assay. (*p<0.05 by Student's One-Tailed T-Test of increased activity, n=3-6, Synergy values <1 are evidence of effect)

Table of data

| | | | | | Synergy, | |
|---|---|---|---|---|---|---|
| | | Ave. + | | | Highest | Synergy, |
| | | EdU | | | Single | Response |
| Condition-mouse | N | Count | SD | p-value | Agent | additivity |
| FM | 6 | 165 | 22 | — | — | — |
| DS10-D3 | 3 | 204 | 23 | — | — | — |
| FGF17 | 3 | 476 | 170 | — | — | — |
| 0.01 ug/mL | | | | | | |
| FGF17 | 3 | 1618 | 508 | — | — | — |
| 0.1 ug/mL | | | | | | |
| DS10-D3 + 0.01 | 3 | 3197 | 468 | 3.9E−3 | 0.145 | 0.213 |
| ug/mL FGF17 | | | | | | |
| DS10-D3 + 0.1 | 3 | 6558 | 307 | 3.8E4 | 0.2247 | 0.278 |
| ug/mL FGF17 | | | | | | |

Table of data

| | | | | | Synergy, | |
|---|---|---|---|---|---|---|
| | | Ave. + | | | Highest | Synergy, |
| | | EdU | | | Single | Response |
| Condition-human | N | Counts | SD | p-value | Agent | additivity |
| FM | 3 | 205 | 28 | — | — | — |
| DS10-D2 | 3 | 197 | 18 | — | — | — |
| FGF17 0.1 ug/mL | 3 | 349 | 62 | — | — | — |
| FGF17 0.1 ug/mL + | 3 | 782 | 85 | 1.9E-3 | 0.45 | 0.70 |
| DS10-D2 | | | | | | |

### Example 30—Intramuscular Administration of FGF17 Promoted the Regeneration of Muscle in BaCl2 Injured Old Mice Model

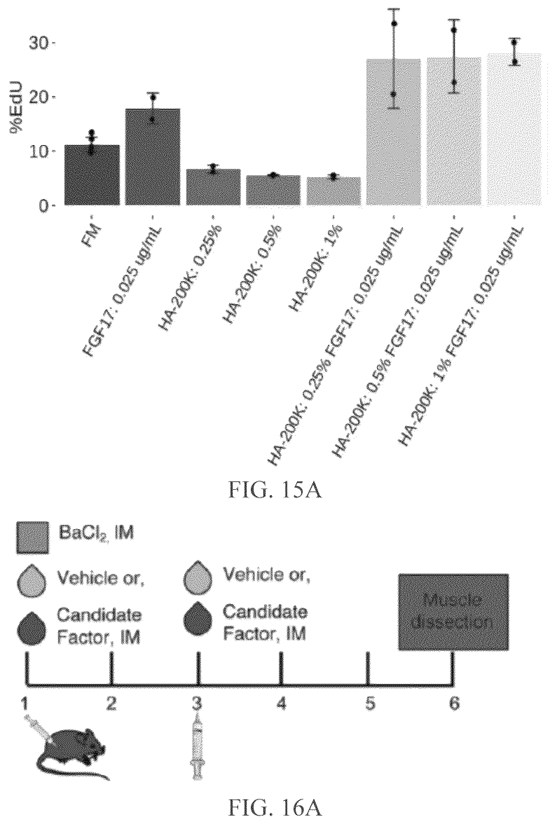

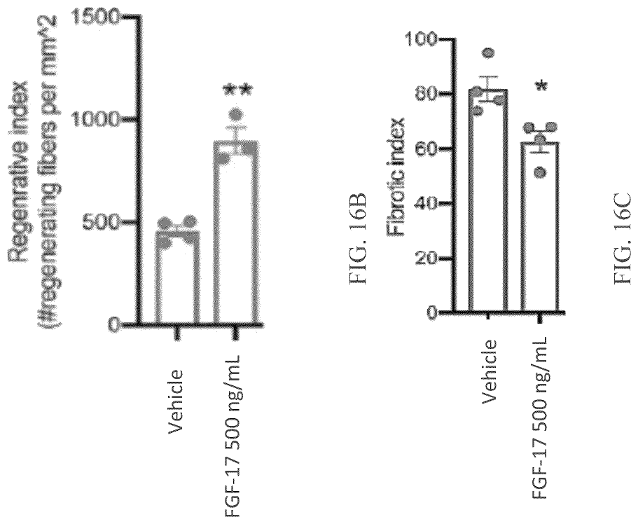

FIG. 16A) Experiment overview. Intramuscular injection of 1.2% of BaCl2(7 ul/TA) was used to generate chemical injury in the TAs of 78 weeks old mice. FGF17 (500 ng/mL) was administered via intramuscular injection after 2 h and 48 h of muscle injury. FIG. 16B) Quantification of the regenerative index calculated as the number of newly regenerated fibers per mm\hat{ }of injury area. Regenerated fibers were identified as fibers with central nuclei,****p<0.0001 (unpaired t-test). FIG. 16C) Histogram showing the fibrotic index calculated as the percentage of the fibrotic area. *p=0.0186 (unpaired t-test).

### Example 31—Systemic Administration of FGF17 Protects Against Dexamethasone Induced Muscle Atrophy

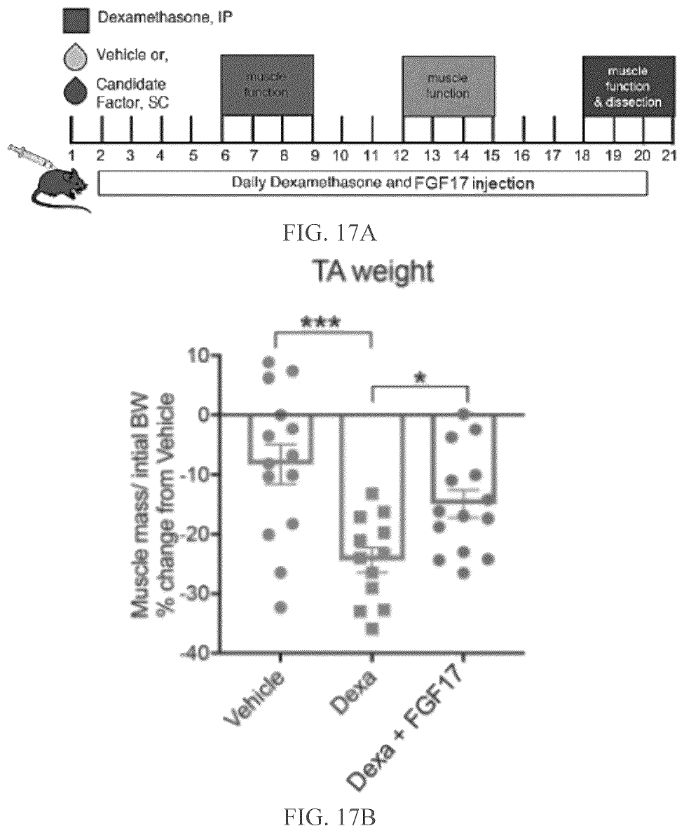

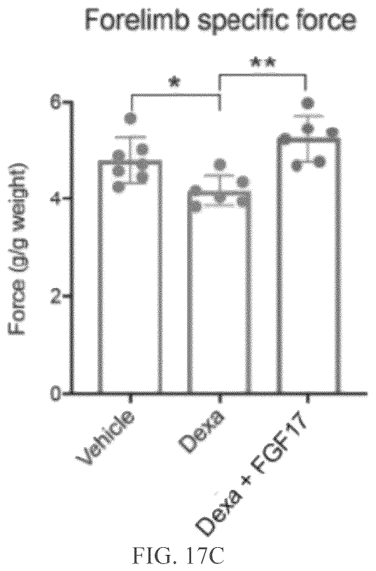

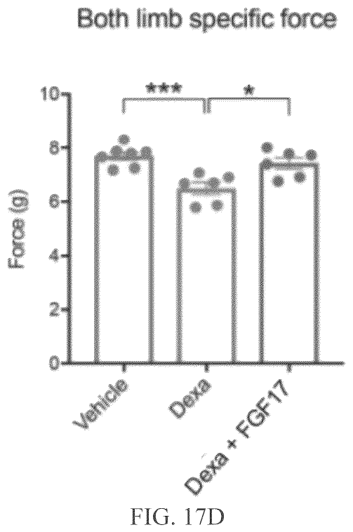

FIG. 17A) Experiment overview and groups. Dexamethasone (25 mg/kg i.p.) was administered to 12 weeks old mice for 20 days simultaneously with a subcutaneous injection of FGF17 (0.5 mg/kg). Muscle weight was assessed on Day 21. Forelimb grip strength and both limb grip strength were measured on Day 7, 13 and 21 FIG. 17B) TAs muscle weight over initial body weight shown as the percentage change from vehicle. ***p=0.0005, *p=0.0499. Forelimb force measured on Day 21, histogram shows the specific forelimb force calculated as the ratio of forelimb force in N over the weight in g, *p=0.0458, **p=0.0014 FIG. 26D17C) Both limb force measured on Day 21 calculated as the ratio of both limb force in N over the weight in g***p=0.001 *p=0.0102. One way Anova corrected for multiple comparisons using Tukey method was used to compare data.

### Example 32—Treatment for Chondrocyte Proliferation in Cartilage Injury and Osteoarthritis And FGF17 Induction of Chondrocyte Proliferation

RNA expression shows FGF17 receptor was expressed on cartilage-associated cells.) Bar graph quantitation of % EdU+ human chondrocyte in response to FGF17. Chondrocytes were cultured for 48 h in the presence of FGF17 at indicated dose. FGF18 was added at 0.1 ug/mL as a positive control. Fresh media and FGF17 was added every 24 h. Table of % EdU+ chondrocyte and p-values depicted. Mean+̅S.D. (*p<0.05 by Tukey Honest Significant Difference T-test, n=2-3)

TABLE
FGFR1 RNA Expression (TPM)

| Cell Type | FGFR1 |
|---|---|
| Preadipocyte (Subcutaneous) | 54.92 |
| Chondrocyte | 152.59 |
| Osteocyte | 202.57 |
| Tenocyte | 167.01 |

Table of data

| Condition | n | % EdU | sd | adj-p-val |
|---|---|---|---|---|
| Vehicle | 3 | 0.744 | 0.247 | |
| FGF18: 0.1 ug/mL | 3 | 6.039 | 1.637 | 1.29E−03 |
| FGF17: 0.1 ug/mL | 2 | 4.675 | 0.198 | 1.92E−04 |
| FGF17: 0.20 ug/mL | 2 | 9.085 | 0.094 | 7.85E−06 |
| FGF17: 0.40 ug/mL | 2 | 16.773 | 0.621 | 5.42E−07 |

### Example 33—Myogenic Activity Measurement Assay In Vitro

### Mouse Myoblast Proliferation Assay

Reduced regeneration from an individual's tissue progenitor cells is a hallmark of age or disease related dysfunction, therefore assays that measure mitogenic capacity in tissue progenitor cells serve as a read-out for potential success of a treatment. Measuring the increased proliferation rate, degree of differentiation, and cellular survival of treated mouse or human muscle progenitor cells will provide good basis for potentially therapeutic regenerative factors

for treating individuals who have suffered illness, injury, or who possess genetic or developmental defects leading to premature tissue loss, wasting, or weakening.

Mouse muscle progenitor cells (early passage myoblasts) were cultured and expanded in mouse growth medium: Ham's F-10 (Gibco), 20% Bovine Growth Serum (Hyclone), 5 ng/mL FGF2 and 1% penicillin-streptomycin on Matrigel coated plates (1:300 matrigel:PBS), at 37° C. and 5% CO2. For experimental conditions, cells were plated at 40,000 cells/well on Matrigel coated 8-well chamber slides in 250-500 μL medium per well (1:100 matrigel:PBS) in mouse fusion medium: DMEM (Gibco)+2% horse serum (Hyclone). One hour after plating, mouse myoblasts were treated with 50% respective medias: Mouse myoblasts were cultured for 24 hours in the above conditions, at 37° C. in 10% CO2 incubator. BrdU (300 μM) in DMSO was added for 2 hours prior to fixation with cold 70% ethanol and stored at 4° C. until staining.

### Quantifying Regenerative Index

Following permeabilization in PBS+0.25% Triton X-100, antigen retrieval was performed. Primary staining was performed with primary antibodies including: a species-specific monoclonal antibody for mouse anti-embryonic Myosin Heavy Chain (eMyHC, hybridoma clone 1.652, Developmental Studies Hybridoma Bank) and Rat-anti-BrdU (Abcam Inc. ab6326). Secondary staining with fluorophore-conjugated, species-specific antibodies (Donkey anti-Rat-488, #712-485-150; Donkey anti-Mouse-488, #715-485-150. Nuclei are visualized by Hoechst staining. Using the Hoechst stain to tally cell numbers, the percent of cells positive for BrdU and eMyHC were tabulated and reported.

### FGF17-hFcm Promotes Proliferation of Mouse Myoblasts.

Suspension CHO cells were transiently transfected with either the empty control plasmid or the FGF17-hFcm encoding plasmid. After four days, the culture supernatants were collected and added into the culture of mouse myoblast cells for 48 hours, followed by a 2 hour EdU pulse prior to fixation. The percentage of EdU+ mouse myoblasts treated with the culture supernatant of CHO cells expressing FGF17-hFcm is significantly higher than the percentage of EdU+ mouse myoblasts treated with either vehicle control or the culture supernatant of CHO cells expressing the empty control vector (One-Way ANOVA Tukey Honest Significant Difference, n=2-6).

Suspension CHO cells were transiently transfected with the FGF17-hFcm encoding plasmid. After four days, the culture supernatants were collected and FGF17-hFcm was affinity-purified by Protein A membrane column. The purified FGF17-hFcm was added into the culture of mouse myoblast cells for 48 hours, followed by a 2 hour EdU pulse and fixation. The percentage of EdU+ mouse myoblasts treated with the purified FGF17-hFcm is significantly higher than the percentage of EdU+ mouse myoblasts treated with the culture supernatant of CHO cells expressing the empty control vector (One-Way ANOVA Tukey Honest Significant Difference, n=2-6).

### Example 34—Myogenic Gene Profiling for Pro-Regenerative Factors

Expression of myogenic factors Pax7, Myf5, Myod1, and Myog are key indicators of the functional status of muscle progenitor cells. Factors upregulating of Pax7 and Myf5 indicate rejuvenation of proliferative progenitor cells whereas upregulation of Myod1 and Myog are indicative of muscle myofiber regeneration. A read-out of these gene expressions will provide potential success for any given polypeptide comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein. Measuring myogenic genes in mouse or human muscle progenitor cells treated with factors will provide a good characterization of the therapeutic effect for treating individuals who have suffered injury, or who possess genetic or developmental defects leading to premature tissue loss, wasting, or weakening. As a control, the assay will also be performed on proteins purified from differentiated cells, which result in no in myoblast proliferation, cultured in medium conditioned by differentiated cells, or purified heparin-associated fractions.

RNA was isolated from each well (RNeasy Mini Kit, Qiagen) and cDNA was obtained by reverse-transcription (High Capacity Reverse Transcription Kit, Thermo Fisher Scientific). Real-time quantitative PCR was performed using QuantStudio3 (Thermo Fisher).

Aged human myoblasts were cultured in well plates. Culturing the cells with the different medias resulted in differential induction of myogenic gene expression. All factors resulted in changes in at least one myogenic receptor gene at 48 hours and 72 hours when compared to cells cultured in fusion media, as depicted in Table 4. Cells that had been cultured with IGF2 had increases in levels of MYOG at 48 hours and levels of MYOD at 72 hours. Table 4: Myogenic transcription factor fold change increase in myoblasts cultured with IGF2

| Con- | MYF5 - | MYOD1 - | MYOG - | MYF5 - | MYOD1 - | MYOG - |
|---|---|---|---|---|---|---|
| dition | 48 h | 48 h | 48 h | 72 h | 72 h | 72 h |
| FM | 1.04 | 1.001 | 1.013 | 1.023 | 1.055 | 1.092 |
| IGF2 | 0.409 | 0.519 | 5.756 | 0.708 | 5.723 | 0.018 |

### Myogenic Gene Profiling in Human or Mouse Progenitor Cells

Human or mouse muscle progenitor cells will be plated and cultured as described above for myogenic activity testing. One hour after plating, myoblasts will be treated with respective factors. Myoblasts are analyzed for expression of Pax7, Myf5, Myod1, and Myog to characterize the regenerative effect of treatment with polypeptides comprising an FGF17, IGF2, or BMP7 amino acid sequence and will be tested to characterize the effects an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans.

### Example 35—In Vivo Testing of Stem Cell Secreted Factors

Multiple in vivo models of muscle degeneration will be tested. Given that polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans described herein have regenerative properties in in vitro models, these in vivo models will show that similar regenerative and proliferative effects in the context of intact organ systems.

### Acute Injury Model

The experimental groups will be: C57BL/6J male mice, N=18; Young: 12-13 week old (3-month-old) mice, n=6; Aged: 77-78 week old (18-month-old) mice, n=12. This design will be used to test any single factor identified and validated in in vitro assays or polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans.

On Day 0, mice will be weighed and undergo muscle injury with focal injection of barium chloride (BaCl2,10 μL, 1.2% w/v in saline, Sigma-Aldrich) in the Tibialis anterior (TA; Day 0) of both the right and left hindlegs. Injections of vehicle or factor A (0.1 mg/kg) will be co-administered intramuscularly (i.m) following the BaCl2 into the TA injured hindleg sites, and again 48 hours later on day 2 (i.m.) into the TA injured hindleg sites. Also on day 2, BaCl2 (Ctx; 10 μL, 1.2% w/v in saline, Sigma-Aldrich) was injected into the Gastrocnemius (GA, Day 2, i.m.) muscles of both right and left hind legs. Injections of vehicle or a factor will be sequentially administered (i.m.) following the BaCl2 into the TA hindleg sites post-injury, and again 48 hours later on day 4 (i.m.) into the GA injured hind leg sites. Bromodeoxyuridine (BrdU) was be administered (100 mg/kg, i.p.) once daily for 3 days, day 2-4, before sacrifice to label proliferating cells.

On day 5, animals will be sacrificed, and animal weight recorded followed by collecting 0.5 ml of terminal blood via cardiac puncture which was processed to plasma and stored at 80° C. We then perfuse the animal with 1×PBS, carefully dissect the skin from the GA/TA muscles of each hind leg and took photos (prior to excision). After excision of exclusively the GA or TA muscle, excised tissue is photographed, weighed, then placed into 25% sucrose in PBS at 4° C. for 4 hr rinsed in 1×PBS, immersed in Tissue-TEK OCT and rapidly frozen before storing the muscles tissues frozen at 80° C. Cryosectioning and H&E will be performed to ensure muscle injury site was appropriately visualized. Muscle tissue composition from new skeletal muscle fibers, fibrotic tissue, and adipose (fat), will be measured. Muscle regeneration, as defined as the number of number of new myofibers with centrally located nuclei per millimeter, fibrosis as defined as the area of fibrotic scarring, size of the fibers, as defined as the width and area, adipose tissue, as defined by the amount of fat surrounding the muscle, will be measured to assess level of regeneration.

### Sarcopenia/Chronic Administration Model

The experimental design is C57BL/6J male mice, N=18; Young: 12-13 week old (3-month-old) mice, n=6; Aged: 77-78 week old (18-month-old) mice, n=12, as depicted in Table 4. This design can be used to test any single factor identified and validated in in vitro assays or complex mixtures of 2 or more factors or synergistic small molecules.

On Day 0, mice will have the following in vivo healthspan measurements will be performed over 1 day as a baseline for age-based parameters: Weight, running wheel performance, grip strength, and horizontal bar. Each assay should be run for 4 trials per assay per animal. These healthspan assays will be repeated on day −1. After one day of rest on day −9, mice will begin 1× daily injections (0.1 mg/kg) of vehicle or factor A for the remainder of the experiment until sacrifice (days −8 to +5, 13 days of dosing). On day −4, 6 days after dosing begins, mice will undergo a repeat of the healthspan assays. On day 0, 5 days prior to sacrifice, mice will undergo muscle injury with focal injection of cardiotoxin (Ctx; 10 μg, Sigma-Aldrich) in the Tibialis anterior (TA; day 0) of the right hindleg only. On day 2, the Gastrocnemius (GA; day 2) muscle of the right hind leg will then receive cardiotoxin (Ctx; 10 μg, Sigma-Aldrich). BrdU will be administered (100 mg/kg, i.p.) once daily for 3 days, day 2-4, before sacrifice. On day +5, prior to take-down, the animals will have an in vivo incapacitance assay run. On day +5, animals will be sacrificed, and animal weight recorded. We will Collect 0.5 ml of blood via cardiac puncture, process to plasma and store plasma samples at 80° C. The animals will then be perfused with 1×PBS. Carefully dissect the skin from the GA/TA muscles of each hind leg and take photos (prior to excision). After excision of exclusively the GA or TA muscle, we will weigh the muscles, then place muscles into 25% sucrose in PBS at 4° C. for 4 hours, then rinse the muscles in 1×PBS, adding Tissue-TEK OCT and storing the muscles tissues frozen at 80° C. Perform cryosectioning and H&E, ensuring muscle injury site is appropriately visualized. Carefully excising the inguinal white adipose tissue (WAT) will be weighed.

Muscle tissue composition, from new skeletal muscle fibers, fibrotic tissue, and adipose (fat), will be measured. Muscle regeneration, as defined as the number of number of new myofibers with centrally located nuclei per millimeter, fibrosis, as defined as the area of fibrotic scarring, size of the fibers, as defined as the width and area, adipose tissue, as defined by the amount of fat surrounding the muscle, will be measured to assess level of regeneration. Weights of the animals during the duration of treatment, as well as healthspan assays including performance on a running wheel (speed, distance, duration), grip strength, and performance on a horizontal bar will take into account the phenotypic outcomes of treatment of the aged animals systemically with the polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans.

The horizontal bar test will be performed as described previously (Malinowska et al. 2010) at 8 months (n=6 WT, n=7 MPS IIIB) and 10 months (n=3 WT, n=4 MPS IIIB) of age. In brief, a 300-mm metal wire, 2 mm in diameter, was secured between two posts 320 mm above a padded surface. The mouse will be allowed to grip the center of the wire and the time to fall or reach the side was recorded, and after 2 minutes the test was stopped. Crossing the bar in x seconds will be scored as 240-x, remaining on the bar will be scored as 120, and falling off the bar after y seconds will be recorded as the value of y. The test will be repeated three times as a practice run followed by a 10-min rest prior to three tests where the score was recorded.

Animals will also have better healthspan outcomes: reduced weight, fat composition, scar tissue around muscles, increased running speed, duration, and distance, increased grip strength, and enhanced performance on the horizontal bar test.

### Genetically Obese Muscle Dystrophy Model

Genetically obese (ob/ob) mice will be injected with BaCl2 on day 0 in the TA muscle. 3 mice will be treated with vehicle only, 3 mice will be injected with the hPSC factors and 3 mice will be treated with FGF19 (positive control) on day 0 and day 2. On day 5, the mice will be euthanized, the TA muscles perfused with PBS, and dissected. Muscles will be then analyzed for regenerative index and fibrotic index.

### Atrophy Model

The experimental design is C57BL/6J male mice with daily administration of Vehicle, N=7; dexamethasone (25 mg/kg i.p.), N=6, or dexamethasone+treatment article, N=6, for 20 days. This design can be used to test any single factor identified and validated in in vitro assays or complex mixtures of 2 or more factors.

On day 21, animals are sacrificed, and animal weight recorded. 0.5 ml of blood is collected via cardiac puncture and processed to plasma for storage of plasma samples at −80° C. The animals are perfused with 1×PBS. After carefully dissecting the skin from the GA/TA muscles of each hind leg, photos are taken, followed by excision of exclusively the GA or TA muscle, weighing the muscles, then flash freezing in isopentane at −80 C.

### Methods of Testing Muscle Strength, Endurance and Function

Forelimb and Both limb grip strength test: After 30 min acclimation, the mice are introduced to the grip strength meter. For forelimb grip strength, the mice held by the tail are allowed to grasp the grip bar with only its forelimbs. For both limb measurements the mice are placed on the grid and allowed to grasp the grid with both limbs. The force generated by each mouse is calculated as the average of 5-6 measurements.

Limb endurance test: Mice are allowed to discover and acclimate the rodent treadmill environment through 2 training sessions of 10 minutes each at 10 m/min on separate days prior to the endurance test. For the endurance test, mice are placed in the individual lanes of the rodent treadmill. The speed is gradually increased at 2 m/min until exhaustion is reached. Exhaustion is defined as a mouse staying on a grill electrified to deliver a shock of 2 Hz, intensity 5 for 3-5 seconds.

In vivo tetanic force measurement: Mice are under anesthesia using regulated delivery of isoflurane during the whole process. Following anesthetization, the animal is placed onto a heated chamber with the foot secured on the foot pedal of an Aurora force transducer. The 2 electrodes are placed specifically to stimulate the sciatic nerve. The force generated by the ankle torsion of the animal's hind limb, as opposed to direct force is measured in response to a series of stimulation that includes 50, 100, 150 and 200 Hz.

In situ tetanic force measurement: This experiment is performed using Aurora force measurement. Mice are under anesthesia during the whole process. A small incision in the skin around the Anterior Tibialis exposes the Achilles tendon which is connected via surgical suture to the Aurora force transducer through a hook. The force generated by the muscle in response to a series of stimulation that includes 50, 100, 150 and 200 Hz by 2 electrodes placed on the anterior tibialis is recorded.

### Example 36—Mitogenic Polypeptide Stability In Vivo Assayed by Bioavailability and Pharmacokinetics

### Bioavailability in Tissues

The bioavailability of the therapeutic polypeptides will be assessed in the target tissues in young mice (10-12 weeks old) and old mice (78 weeks old). For this experiment, 1 cohort of young mice (10-12 weeks old; N=24) and 1 cohort of old mice (78 weeks old; N=24) will receive 1 subcutaneous (SC) injection of a therapeutic composition. 4 young mice (10-12 weeks old; N=6) and 4 old mice (78 weeks old; N=6) will receive 1 SC injection of Vehicle and used as control. 4 mice from each cohort will be euthanized after 30 minutes, 1 hour, 1.5 hours, 2 hours, or 4 hours. At each time point blood will be collected by heart puncture followed by harvesting select tissues, such as the tibialis anterior, gastrocnemius, quadriceps, heart and diaphragm. The detection and quantitation of the administered therapeutic polypeptides will be detected by enzyme-linked immunosorbent assay (ELISA). The level of therapeutic polypeptides will be compared to the samples collected from mice injected with vehicle to determine tissue level bioavailability.

### Pharmacokinetics of Engineered Mitogenic Polypeptides

Murine pharmacokinetics (PK) represents the absorption, distribution, metabolism, and elimination of drugs from the body. The pharmacokinetic profile of the therapeutic polypeptides will be determined in young mice (10-12 weeks old) and old mice (78 weeks old). For this experiment, 2 routes of administration will be investigated, including SC and intravenous (IV) injection in both young (10-12 weeks old) and old mice (78 weeks old). Six time points for each group 5, 15, 30, 60, 90 and 120 minutes will be assessed using end-point or serial sampling. At least 4 animals will be used for each time point/group/route. Engineered mitogenic polypeptides concentrations in the samples will be measured by LC-MS/MS or ELISA. Various pharmacokinetics will be calculated as well as the absorption/elimination dynamics following different routes of administration.

### Example 37—Additional Tests for Regenerative Factors

Mechanistic insight into polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans combinations pathway of action will be gained by establishing and screening against a panel of assays for cellular age. Assays include measurements of reactive oxygen species (ROS) production or tolerance cytoplasmically and in the mitochondria, telomerase activity, measurements of proteostasis capacity via lysosomal, autophagy, and proteasomal routes, epigenetic re-patterning, and cellular energy balance (e.g., ATP/ADP and NAD/NADH ratios). Many of these assays leverage high-throughput automated microscopy to make these measurements in a variety of cell types, including fibroblast, endothelial cells, mesenchymal stem cells, and chondrocytes. Collectively these metrics can inform both the pathway and the mechanisms by which the heparin-associated hPSC secretome or its individual components enact their regenerative effects. These deep profile vectors can be crucial for approaching combinations of factors rationally, and for machine learning predictions.

To test the cellular effects of secretomes toward reversing the hallmarks of aging, high-throughput automated imaging and quantification of single cells to achieve deep population level statistical power can be employed. Cellular component state profiles of Young, Aged, and Aged+Treatment in human fibroblasts and epithelial cells, myoblasts, mesenchymal stem cells, chondrocytes, and neural progenitor cells will be compared. Some examples of tests and methods include:

Epigenetic reprogramming: repressive mark H3K9me3, the heterochromatin-associated protein HP1γ, nuclear lamina support protein LAP2α.

Nuclear membrane Folding/Blebbing: immunofluorescence of the nuclear membrane protein Lamin A/C.

Proteolytic Activity: Cleavage of fluorescent-tagged chymotrypsin like substrate corresponds to proteasome 20S core particle activity. Wells will be first stained with PrestoBlue Cell Viability dye (Life Technologies) for 10 minutes. Well signals will be read using a TECAN fluorescence plate reader as a measure of cell count. Then cells will be washed with HBSS/Ca/Mg before switching to original media containing the chymotrypsin like fluorogenic substrate LLVY-R110 (Sigma) which is cleaved by the proteasome 20S core particle. Cells will be then incubated at 37° C. in 5% C02 for 2 hours before signals will be again read on the TECAN fluorescence plate reader. Readings will be then normalized by PrestoBlue cell count.

Formation of autophagosomes: Autophagosome number and volume will be measured by staining with CellTracker Deep Red (Sigma). The cells will be then incubated at 37° C. in 5% C02 for 20 minutes, washed 2 times using HBSS/Ca/Mg, and stained for 15 minutes using CellTracker Deep Red cell labeling dye. Cells will be then switched to HBSS/Ca/Mg for single cell imaging using the Operetta High Content Imaging System (Perkin Elmer).

Energy Metabolism: ATP in the cells is measured using colorimetric assay using an ATP assay kit (ab83355; Abcam, Cambridge, MA) following manufacturer's instructions. Cells will be washed in cold phosphate buffered saline and homogenized and centrifuged to collect the supernatant. The samples will be loaded with assay buffer in triplicate. ATP reaction mix and background control (50 μL) is added to the wells and incubated for 30 min in dark. The plate is read at OD 570 nm using SpectraMax M2e (Molecular Devices, Sunnyvale, CA). The mean optical density is used to estimate of the intracellular ATP concentration relative to the standard curve.

Mitochondrial Activity: To measure Mitochondria Membrane Potential, cells will be washed twice with Ham's F10 (no serum or pen/strep). Subsequently, MuSCs will be stained with MitoTracker Green FM (ThermoFisher, M7514) and DAPI for 30 minutes at 37° C., washed three times with Ham's F10, and analyzed using a BD FACSAria III flow cytometer.

Mitochondrial ROS Measurement. Cells will be washed with HBSS/Ca/Mg and then switched to HBSS/Ca/Mg containing MitoSOX (Thermo), a live cell permeant fluorogenic dye that is selectively targeted to mitochondria and fluoresces when oxidized by superoxide. Cells will be incubated for 10 minutes at 37° C. in 5% C02. Cells will be then washed twice with HBSS/Ca/Mg, and stained for 15 minutes using CellTracker Deep Red. Finally, cells will be imaged in fresh HBSS/Ca/Mg using the Operetta High Content Imaging System (Perkin Elmer).

Deregulated Nutrient Sensing: levels of SIRT1 will be measured.

Senescence: Senescence-associated beta-galactosidase staining is measured in cells washed twice with PBS then fixed with 15% Paraformaldehyde in PBS for 6 minutes. Cells will be rinsed 3 times with PBS before staining with X-gal chromogenic substrate, which is cleaved by endogenous Beta galactosidase. Plates will be kept in the staining solution, Parafilmed, to prevent from drying out, and incubated overnight at 37° C. with ambient CO2. The next day, cells will be washed again with PBS before switching to a 70% glycerol solution for imaging under a Leica brightfield microscope.

Secretome of the cells: Mass-Spec or O-Link for inflammatory cytokines profiles.

Soft Tissue Deposition: Immunofluorescence for SOX9, MMP3, MMP13, and COL2A1 expression, the decrease of which is characterized by cartilage loss, pain, cleft-lip, and joint destruction.

### Example 38—the Purified IGF2-hFcm Promoted Differentiation of Myoblast Cells

Suspension CHO cells were transiently transfected with the IGF2-hFcm encoding plasmid. After four days, the culture supernatants were collected and IGF2-hFcm was affinity-purified by Protein A membrane column. The purified IGF2-hFcm was added into the culture of human myoblast cells. Myosin heavy chain (MyHC) was immunostained and imaged by a fluorescent microscope. After quantification of the stained MyHC, the percentage area of MyHC was calculated as the percent of pixels within the field that are illuminated above background in the stained channel. The percentage of EdU of mouse myoblasts treated with the purified IGF2-hFcm is significantly higher than the percentage of EdU of mouse myoblasts treated with the culture supernatant of CHO cells expressing the empty control vector. Significance was determined by a p-value less than 0.05 by the one-way ANOVA Tukey Honest Significant Difference test.

TABLE 5
IGF2 promoted differentiation of myoblast cells

| Condition | % MyHC | SD | p_value |
|---|---|---|---|
| Vehicle control | 1.787 | 0.186 | |
| 33 nM IGF2-hFcm | 3.734 | 0.790 | 0.012 |
| 66 nM IGF2-hFcm | 5.922 | 0.795 | 3.20E−05 |
| 133 nM IGF2-hFcm | 7.568 | 0.538 | 1.46E−06 |

This example found that the IGF2-fusion protein was able to induce cell proliferation. The IGF2-fusion protein shares in vitro properties with the HAPs, which is suggestive of shared in vivo properties.

### Example 39—Modelling Treatment of a Muscular Dystrophy with an IGF2 Composition In Vitro

Muscular dystrophies (MD) encompass a variety of muscular degeneration diseases typically due to genetic mutations in genes encoding proteins responsible for forming and stabilizing skeletal muscle. The phenotypic consequence of these genetic mutations is the progressive loss of muscle mass and strength over time, similar to sarcopenia but with different underlying causes. As HAPs provided phenotypic improvements on sarcopenic muscle, we tested for similar improvements in a model for MD.

IGF2 was tested individually for its ability to promote proliferation and/or fusion of human muscle progenitor cells from an individual with myotonic dystrophy type 1 (hMD)—a muscular dystrophy caused by mutations in the DMPK1 gene. The effect of IGF2 on myogenic activity was assayed in biological triplicate across a range of concentrations centered around expected physiological levels by adding each factor to hMD myoblasts for 72 hours with daily media changes (DMEM+2% horse serum) and a second pulse of factors at the first media change. After 72 or 96 hours, cells were pulsed for 2-5 hours with EdU (30 uM), ethanol fixed, stained with Hoescht 3342, immunostained for proliferation—as measured by the percent of cells staining positive for EdU (% EdU)-, and immunostained for differentiation—as measured by the increase in cellular area staining positive for embryonic myosin heavy chain (% eMyHC) relative to the negative controls, which received media and vehicle only. Wells were imaged on a Keyence BZ-100 at 4×, the images quantified in Cell Profiler, and the statistics were computed in R. Additionally, RNA was extracted from myoblast and select transcript abundances quantified by qPCR. depicts IGF2 treatment promoted proliferation and differentiation respectively in DM1 human myoblast (32 year old caucasian female) cells. depict IGF2 enhanced MYH3, CKM, and ATP1B1 expression in DM1 human myoblast (32 year old caucasian female) cells.

### Example 40—Systemic Administration of Therapeutic Polypeptides Reverses Sarcopenia and Protects From Muscle Injury

A daily subcutaneous injection of therapeutic polypeptides or vehicle only is administered to 78 week old mice for 14 days. IGF2 is injected at a concentration of 100-1000 μg/kg. In some experiments, treatment groups receive a single therapeutic factor while in other experiments, treatment groups receive a combination of factors. At 7 days, muscle function is assessed using forelimb grip strength and both grip strength. On day 12, 13 and 14, groups 1 and 2 are injected with BrdU intraperitoneally. On days 13-15, all mice are assessed for grip strength and an endurance test to determine max distance and max speed and tetanic force.

At 15 days, mice in groups 1 and 2 are euthanized and the muscles are analyzed for markers of proliferation and fibrosis. At 15 days, an intramuscular injection of 1.2% of BaCl2 (7 ul/TA) is used to generate chemical injury in the TAs of group 3 and group 4. Mice from groups 3 and 4 continue to receive a therapeutic polypeptide injected subcutaneously on days 15-21. They also receive BrdU injections intraperitoneally on days 19, 20, and 21. On day 21, the TA muscles are tested for in situ tetanic force. The TA muscles are dissected and assessed for signs of proliferation and fibrosis.

### Example 41—Systemic Administration of Fusion Polypeptides Reversed Induced Muscle Atrophy

12-week-old mice are divided into 3 treatment groups: group 1 which receives injections only of the vehicle, group 2 which receives injections of dexamethasone, and group 3 which receives injections of dexamethasone and IGF2 fusion polypeptide. Dexamethasone (25 mg/kg i.p.) is administered for 14 days simultaneously with a subcutaneous injection of IGF2 fusion polypeptide.

At 7 days, mice are assessed for forelimb and both limb grip strength. At days 13-15, mice are assessed for grip strength, in vivo tetanic force, and undergo a treadmill endurance test to determine max speed and max distance.

### Example 42-Systemic Administration of IGF2 Fusion Polypeptide Predicted to Improve Muscle Atrophy in Genetically Obese Mice

Thirteen-week old genetically obese mice (ob ob) will be injected subcutaneously with an IGF2 fusion polypeptide for 14 days. At day 7, forelimb and both grip strength will be measured. BrdU is injected on days 12, 13 and 14. On days 13, 14 and 15, forelimb and both limb grip strength and in vivo tetanic force will be tested, and an endurance test to determine max distance and max speed is performed. At 14 days, the mice will be euthanized, and the TA muscles dissected. Muscle weight and proliferation will be analyzed.

### Example 43—Systemic Administration of IGF2 Fusion Polypeptide Predicted to Reverse of Slow Down Dystrophic Features in 70 Weeks Old Mdx Mice

Another class of human myopathies in need of treatment are the genetic abnormality induced muscular dystrophies, among which Duchenne muscular dystrophy is a rare but fatal case. Old genetically dystrophic (mdx) mice (>15 month old) show similar features to the human Duchenne muscular dystrophy (DMD), notably, a decrease in muscle regeneration leading to muscle wasting. Treatment with IGF2 fusion polypeptide can reverse the dystrophic features of old mdx mice. During the acclimation period, the weight, Forelimb and both limb grip strength as well as in vivo tetanic force will be assessed to determine the baseline strength of each mouse. 70 week dystrophic mice (mdx) are injected with the IGF2 fusion polypeptide subcutaneously for 14 days. At day 7, forelimb and both grip strength are measured. BrdU is injected on days 12, 13 and 14. On days 13, 14, and 15, forelimb and both limb grip strength and in vivo tetanic force are tested, and an endurance test to determine max distance and max speed is performed. The right tibialis anterior and gastrocnemius will be collected, immersed in Tissue-TEK OCT and then flash frozen in chilled isopentane bath precooled in liquid nitrogen and stored at −80° C. Tissue will be sectioned and stained for Laminin to determine the cross sectional area (CSA) of muscle fibers, for eMyHC to measure new fiber formation and for BrdU to assess the proliferation rate. The left anterior tibialis and gastrocnemius will be collected and flash frozen in liquid nitrogen for molecular analysis that include qPCR and western blot.

IGF2 is predicted to be effective at a concentration of 10-200 ug/kg.

### Example 44—Systemic Administration of IGF2 Fusion Polypeptide Predicted to Improve the Dystrophic Features in 6 Week Old Mice

Between 3-6 weeks old, the skeletal muscle of mdx mice undergoes severe necrosis followed by an increase in the activation of satellite cells to promote muscle regeneration. Treatment with IGF2 fusion polypeptide described herein can improve the regeneration process and therefore muscle health. During the acclimation period, the weight, Forelimb and both limb grip strength as well as in vivo tetanic force will be assessed to determine the baseline strength of each mouse. 6 week old dystrophic mice (mdx) are injected with the IGF2 fusion polypeptide subcutaneously for 14 days. At day 7, forelimb and both grip strength are measured. BrdU is injected on days 12, 13 and 14. On days 13, 14 and 15, forelimb and both limb grip strength and in vivo tetanic force are tested, and an endurance test to determine max distance and max speed is performed.

Mice will be euthanized. The right tibialis anterior and gastrocnemius will be collected, immersed in Tissue-TEK OCT and then flash frozen in chilled isopentane bath precooled in liquid nitrogen and stored at −80° C. Tissue will be sectioned and stained for Laminin to determine the cross sectional area (CSA) of muscle fibers, for eMyHC to measure new fiber formation and for BrdU to assess the proliferation rate. The left anterior tibialis and gastrocnemius will be collected and flash frozen in liquid nitrogen for molecular analysis that include qPCR and western blot.

IGF2 is predicted to be effective at a concentration of 10-200 μg/kg.

### Example 45—Treatment for Chondrocyte Proliferation in Cartilage Injury and Osteoarthritis

Cartilage can become damaged as a result of a sudden injury or due to gradual wear and tear or inflammation leading to disease states (e.g. osteoarthritis). Chondrocytes secrete the cartilage matrix and preadipocytes, osteocytes and tenocytes are all cell types associated with cartilage.

Preadipocytes, chondrocytes, osteocytes and tenocytes were cultured in well plates. RNA was isolated from each well (RNeasy Mini Kit, Qiagen) and cDNA was obtained by reverse-transcription (High Capacity Reverse Transcription Kit, Thermo Fisher Scientific). Real-time quantitative PCR was performed using QuantStudio3 (Thermo Fisher).

These cartilage-associated cells expressed receptors for polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans. Subcutaneous preadipocytes, chondrocytes, osteocytes and tenocytes all expressed one or more receptors, indicating that these polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans may be able to affect cartilage loss and the progression of joint related injury or disease recovery.

### Example 46—Clinical Testing of Therapeutic Compositions

The purpose of this study is to determine the safety, tolerability, and pharmacokinetics of repeat dosing with multiple dose levels of polypeptides comprising an FGF17, IGF2, or BMP7 amino acid sequence and an amino acid sequence from a heterologous polypeptide or combinations of an Fibroblast Growth Factor Receptor agonist and a glycosaminoglycan, an Insulin-like Growth Factor 1 Receptor (IGF1R) agonist and a short chain fatty acid, and BMP receptor agonists and mTOR activators and/or glycosaminoglycans in healthy individuals or individuals diagnosed with sarcopenia, a muscular dystrophy, or recovery from surgery. In certain embodiments, the muscular dystrophy is myotonic dystrophy. In addition, this study will generate data on the physical function, skeletal muscle mass and strength resulting from treatment with IGF2 fusion polypeptides in such individuals. Individuals will be administered placebo or IGF2 fusion polypeptide compositions and monitored for 25 weeks of study. The following primary and secondary outcome measures will be assessed:

### Primary Outcome Measures:

Safety and tolerability as assessed by various measures such as percent of adverse events per study arm.

### Secondary Outcome Measures:

Plasma Pharmacokinetics (Cmax, Tmax, AUC) [Plasma at 0.5, 1, 1.5, 2, 4, 6, 8, 12 and 24 hrs after dosing.]

Short Physical Performance Battery (SPPB). Change from baseline to week 25.

10-meter walk test. Change from baseline to week 25.

Change in total lean body mass and appendicular skeletal muscle index measured by Dual-energy X-ray Absorptiometry (DEXA) from baseline to week 25.

### Inclusion Criteria:

Diagnosis of sarcopenia, a muscular dystrophy, or recovery from surgery; Low muscle mass as confirmed by DXA; Low gait speed; SPPB score less than or equal to 9; Weigh at least 35 kg; with adequate dietary intake as determined by patient interview. Independently ambulatory to 10 meters.

### Protocol

Patients will be i.v.-administered placebo (5% dextrose solution) or treatment article (in 5% dextrose). Starting on day 1, week 1 and repeated every week (day one of weeks 1 through 25). At the end of week 13 and 25 patients will be assessed by the above methods for improvement. Doses will be selected from a traditional 3+3 design, and selected as the top two-doses that lack dose-limiting toxicity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

TABLE 1
Secretory Signal Sequences

| Seq ID | Sequence | | |
|---|---|---|---|
| No | Name | Description | Sequence |
| 1 | spFGF-17 | Human FGF-17 | ATGGGAGCCGCCCGCCTGCTGCCCAACCTCA |
| | | secretory signal peptide | CTCTGTGCTTACAGCTGCTGATTCTCTGCTGT |
| | | nucleotide sequence | CAA |
| 2 | spTHBS1 | Human THBS1 | ATGGGGCTGGCCTGGGGACTAGGCGTCCTGT |
| | | secretory signal peptide | TCCTGATGCATGTGTGTGGCACC |
| | | nucleotide sequence | |
| 3 | spIGF-2 | Human IGF-2 secretory | ATGGGAATCCCAATGGGGAAGTCGATGCTGG |
| | | signal peptide | TGCTTCTCACCTTCTTGGCCTTCGCCTCGTGC |
| | | nucleotide sequence | TGCATTGCT |
| 4 | spBMP-7 | Human BMP-7 | ATGCACGTGCGCTCACTGCGAGCTGCGGCGC |
| | | secretory signal peptide | CGCACAGCTTCGTGGCGCTCTGGGCACCCCT |
| | | nucleotide sequence | GTTCCTGCTGCGCTCCGCCCTGGCC |
| 5 | spALB | Human Albumin | ATGAAGTGGGTAACCTTTATTTCCCTTCTTTT |
| | | secretory signal peptide | TCTCTTTAGCTCGGCTTATTCC |
| | | nucleotide sequence | |
| 6 | spAZU1 | Human Azurocidin | ATGACCCGGCTGACAGTCCTGGCCCTGCTGG |
| | | secretory signal peptide | CTGGTCTGCTGGCGTCCTCGAGGGCC |
| | | nucleotide sequence | |
| 7 | spBM40 | Human osteonectin | ATGAGGGCCTGGATCTTCTTTCTCCTTTGCCT |
| | | secretory signal peptide | GGCCGGGAGGGCTCTGGCAGCA |
| | | nucleotide sequence | |
| 8 | spGAU | Gaussia luciferase | ATGGGAGTCAAAGTTCTGTTTGCCCTGATCTG |
| | | secretory signal peptide | CATCGCTGTGGCCGAGGCC |
| | | nucleotide sequence | |
| 9 | spFGF-17 | Human FGF-17 | MGAARLLPNLTLCLQLLILCCQ |
| | | secretory signal peptide | |
| | | amino acid sequence | |
| 10 | spTHBS1 | Human THBS1 | MGLAWGLGVLFLMHVCGT |
| | | secretory signal peptide | |
| | | amino acid sequence | |
| 11 | spIGF-2 | Human IGF-2 secretory | MGIPMGKSMLVLLTFLAFASCCIA |
| | | signal peptide amino | |
| | | acid sequence | |
| 12 | spBMP-7 | Human BMP-7 | MHVRSLRAAAPHSFVALWAPLFLLRSALA |
| | | secretory signal peptide | |
| | | amino acid sequence | |
| 13 | spALB | Human Albumin | MKWVTFISLLFLFSSAYS |
| | | secretory signal peptide | |
| | | amino acid sequence | |
| 14 | spAZU1 | Human Azurocidin | MTRLTVLALLAGLLASSRA |
| | | secretory signal peptide | |
| | | amino acid sequence | |
| 15 | spBM40 | Human osteonectin | MRAWIFFLLCLAGRALAA |
| | | secretory signal peptide | |
| | | amino acid sequence | |
| 16 | spGAU | Gaussia luciferase | MGVKVLFALICIAVAEA |
| | | secretory signal peptide | |
| | | amino acid sequence | |

TABLE 2
Therapeutic Sequences

| Seq | Sequence | | |
|---|---|---|---|
| ID No | Name | Description | Sequence |
| 17 | FGF-17 | Full length | ACTCAGGGGGAGAATCACCCGTCTCCTAATTTTAACCAG |
| | | of human | TACGTGAGGGACCAGGGCGCCATGACCGACCAGCTGAG |
| | | FGF-17 | CAGGCGGCAGATCCGCGAGTACCAACTCTACAGCAGGA |
| | | nucleotide | CCAGTGGCAAGCACGTGCAGGTCACCGGGCGTCGCATCT |
| | | sequence | CCGCCACCGCCGAGGACGGCAACAAGTTTGCCAAGCTC |
| | | | ATAGTGGAGACGGACACGTTTGGCAGCCGGGTTCGCATC |
| | | | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCGCCGGACCAAGCGCACACGGCGGCCCCAGCCCC |
| | | | TCACG |
| 18 | FGF- | Human | ACTCAGGGGGAGAATCACCCGTCTCCTAATTTTAACCAG |
| | 17d204- | FGF-17 | TACGTGAGGGACCAGGGCGCCATGACCGACCAGCTGAG |
| | 216 | AA204-216 | CAGGCGGCAGATCCGCGAGTACCAACTCTACAGCAGGA |
| | | deletion | CCAGTGGCAAGCACGTGCAGGTCACCGGGCGTCGCATCT |
| | | mutant | CCGCCACCGCCGAGGACGGCAACAAGTTTGCCAAGCTC |
| | | nucleotide | ATAGTGGAGACGGACACGTTTGGCAGCCGGGTTCGCATC |
| | | sequence | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACC |
| 19 | FGF- | Human | ACTCAGGGGGAGAATCACCCGTCTCCTAATTTTAACCAG |
| | 17d181- | FGF-17 | TACGTGAGGGACCAGGGCGCCATGACCGACCAGCTGAG |
| | 216 | AA181-216 | CAGGCGGCAGATCCGCGAGTACCAACTCTACAGCAGGA |
| | | deletion | CCAGTGGCAAGCACGTGCAGGTCACCGGGCGTCGCATCT |
| | | mutant | CCGCCACCGCCGAGGACGGCAACAAGTTTGCCAAGCTC |
| | | nucleotide | ATAGTGGAGACGGACACGTTTGGCAGCCGGGTTCGCATC |
| | | sequence | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAA |
| 20 | FGF- | Human | ACTCAGGGGGAGAATCACCCGTCTCCTAATTTTAACCAG |
| | 17R204Q | FGF-17 | TACGTGAGGGACCAGGGCGCCATGACCGACCAGCTGAG |
| | K207Q | R204Q | CAGGCGGCAGATCCGCGAGTACCAACTCTACAGCAGGA |
| | | K207Q | CCAGTGGCAAGCACGTGCAGGTCACCGGGCGTCGCATCT |
| | | mutant | CCGCCACCGCCGAGGACGGCAACAAGTTTGCCAAGCTC |
| | | nucleotide | ATAGTGGAGACGGACACGTTTGGCAGCCGGGTTCGCATC |
| | | sequence | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCaGCGGACCcAGCGCACACGGCGGCCCCAGCCCCT |
| | | | CACG |
| 21 | FGF- | Human | ACTCAGGGGGAGAATCACCCGTCTCCTAATTTTAACCAG |
| | 17d197- | FGF17 | TACGTGAGGGACCAGGGCGCCATGACCGACCAGCTGAG |
| | 216 | AA197-216 | CAGGCGGCAGATCCGCGAGTACCAACTCTACAGCAGGA |
| | | deletion | CCAGTGGCAAGCACGTGCAGGTCACCGGGCGTCGCATCT |
| | | mutant | CCGCCACCGCCGAGGACGGCAACAAGTTTGCCAAGCTC |
| | | nucleotide | ATAGTGGAGACGGACACGTTTGGCAGCCGGGTTCGCATC |
| | | sequence | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAG |
| 22 | FGF- | Human | ACTCAGGGGGAGAATCACCCGTCTCCTAATTTTAACCAG |
| | 17K191A | FGF-17 | TACGTGAGGGACCAGGGCGCCATGACCGACCAGCTGAG |
| | K193AS200A | K191A | CAGGCGGCAGATCCGCGAGTACCAACTCTACAGCAGGA |
| | | K193A | CCAGTGGCAAGCACGTGCAGGTCACCGGGCGTCGCATCT |
| | | S200A | CCGCCACCGCCGAGGACGGCAACAAGTTTGCCAAGCTC |
| | | mutant | ATAGTGGAGACGGACACGTTTGGCAGCCGGGTTCGCATC |
| | | nucleotide | AAAGGGGCTGAGAGTGAGAAGTACATCTGTATGAACAA |
| | | sequence | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGGctCAGGcaCAGTTCGAGTTTGTGGGCgCtGCCCCCA |
| | | | CCCGCCGGACCAAGCGCACACGGCGGCCCCAGCCCCTC |
| | | | ACG |
| 23 | FGF-17- | Full length | ACTCAGGGGGAGAATCACCCGTCTCCTAATTTTAACCAG |
| | linker1- | of human | TACGTGAGGGACCAGGGCGCCATGACCGACCAGCTGAG |
| | hFcm | FGF-17- | CAGGCGGCAGATCCGCGAGTACCAACTCTACAGCAGGA |
| | | linker1- | CCAGTGGCAAGCACGTGCAGGTCACCGGGCGTCGCATCT |
| | | hFcm | CCGCCACCGCCGAGGACGGCAACAAGTTTGCCAAGCTC |
| | | nucleotide | ATAGTGGAGACGGACACGTTTGGCAGCCGGGTTCGCATC |
| | | sequence | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCGCCGGACCAAGCGCACACGGCGGCCCCAGCCCC |
| | | | TCACGGGATCGGGATCGGACAAAACTCACACATGCCCA |
| | | | CCGTGCCCAGCACCTGAAGCTGCCGGGGGACCGTCAGTC |
| | | | TTCCTCTTCCCCCCAAAACCCAAGGACACCCTCATGATC |
| | | | TCCCGGACCCCTGAGGTCACATGCGTGGTGGTGGACGTG |
| | | | AGCCACGAAGACCCTGAGGTCAAGTTCAACTGGTACGT |
| | | | GGACGGCGTGGAGGTGCATAATGCCAAGACAAAGCCGC |
| | | | GGGAGGAGCAGTACAACAGCACGTACCGTGTGGTCAGC |
| | | | GTCCTCACCGTCCTGCACCAGGACTGGCTGAATGGCAAG |
| | | | GAGTACAAGTGCAAGGTCTCCAACAAAGCCCTCGGCGC |
| | | | CCCCATCGAGAAAACCATCTCCAAAGCCAAAGGGCAGC |
| | | | CCCGAGAACCACAGGTGTACACCCTGCCCCCATCCCGGG |
| | | | ATGAGCTGACCAAGAACCAGGTCAGCCTGACCTGCCTG |
| | | | GTCAAAGGCTTCTATCCCAGCGACATCGCCGTGGAGTGG |
| | | | GAGAGCAATGGGCAGCCGGAGAACAACTACAAGACCAC |
| | | | GCCTCCCGTGCTGGACTCCGACGGCTCCTTCTTCCTCTAC |
| | | | AGCAAGCTCACCGTGGACAAGAGCAGGTGGCAGCAGGG |
| | | | GAACGTCTTCTCATGCTCCGTGATGCATGAGGCTCTGCA |
| | | | CAACCACTACACGCAGAAaAGCCTCTCCCTGTCTCCGGG |
| | | | TAAA |
| 24 | FGF- | Human | ACTCAGGGGGAGAATCACCCGTCTCCTAATTTTAACCAG |
| | 17d204- | FGF-17 | TACGTGAGGGACCAGGGCGCCATGACCGACCAGCTGAG |
| | 216- | AA204-216 | CAGGCGGCAGATCCGCGAGTACCAACTCTACAGCAGGA |
| | linker1- | deletion | CCAGTGGCAAGCACGTGCAGGTCACCGGGCGTCGCATCT |
| | hFcm | mutant- | CCGCCACCGCCGAGGACGGCAACAAGTTTGCCAAGCTC |
| | | linker1- | ATAGTGGAGACGGACACGTTTGGCAGCCGGGTTCGCATC |
| | | hFcm | AAAGGGGCTGAGAGTGAGAAGTACATCTGTATGAACAA |
| | | nucleotide | GAGGGGCAAGCTCATCGGGAAGCCCAGCGGGAAGAGCA |
| | | sequence | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCGGATCGGGATCGGACAAAACTCACACATGCCCAC |
| | | | CGTGCCCAGCACCTGAAGCTGCCGGGGGACCGTCAGTCT |
| | | | TCCTCTTCCCCCCAAAACCCAAGGACACCCTCATGATCT |
| | | | CCCGGACCCCTGAGGTCACATGCGTGGTGGTGGACGTGA |
| | | | GCCACGAAGACCCTGAGGTCAAGTTCAACTGGTACGTG |
| | | | GACGGCGTGGAGGTGCATAATGCCAAGACAAAGCCGCG |
| | | | GGAGGAGCAGTACAACAGCACGTACCGTGTGGTCAGCG |
| | | | TCCTCACCGTCCTGCACCAGGACTGGCTGAATGGCAAGG |
| | | | AGTACAAGTGCAAGGTCTCCAACAAAGCCCTCGGCGCC |
| | | | CCCATCGAGAAAACCATCTCCAAAGCCAAAGGGCAGCC |
| | | | CCGAGAACCACAGGTGTACACCCTGCCCCCATCCCGGGA |
| | | | TGAGCTGACCAAGAACCAGGTCAGCCTGACCTGCCTGGT |
| | | | CAAAGGCTTCTATCCCAGCGACATCGCCGTGGAGTGGGA |
| | | | GAGCAATGGGCAGCCGGAGAACAACTACAAGACCACGC |
| | | | CTCCCGTGCTGGACTCCGACGGCTCCTTCTTCCTCTACAG |
| | | | CAAGCTCACCGTGGACAAGAGCAGGTGGCAGCAGGGGA |
| | | | ACGTCTTCTCATGCTCCGTGATGCATGAGGCTCTGCACA |
| | | | ACCACTACACGCAGAAaAGCCTCTCCCTGTCTCCGGGTA |
| | | | AA |
| 25 | FGF- | Human | ACTCAGGGGGAGAATCACCCGTCTCCTAATTTTAACCAG |
| | 17d181- | FGF-17 | TACGTGAGGGACCAGGGCGCCATGACCGACCAGCTGAG |
| | 216- | AA181-216 | CAGGCGGCAGATCCGCGAGTACCAACTCTACAGCAGGA |
| | linker1- | deletion | CCAGTGGCAAGCACGTGCAGGTCACCGGGCGTCGCATCT |
| | hFcm | mutant- | CCGCCACCGCCGAGGACGGCAACAAGTTTGCCAAGCTC |
| | | linker1- | ATAGTGGAGACGGACACGTTTGGCAGCCGGGTTCGCATC |
| | | hFcm | AAAGGGGCTGAGAGTGAGAAGTACATCTGTATGAACAA |
| | | nucleotide | GAGGGGCAAGCTCATCGGGAAGCCCAGCGGGAAGAGCA |
| | | sequence | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGATCGGGATCGGACAAAACTCACA |
| | | | CATGCCCACCGTGCCCAGCACCTGAAGCTGCCGGGGGA |
| | | | CCGTCAGTCTTCCTCTTCCCCCCAAAACCCAAGGACACC |
| | | | CTCATGATCTCCCGGACCCCTGAGGTCACATGCGTGGTG |
| | | | GTGGACGTGAGCCACGAAGACCCTGAGGTCAAGTTCAA |
| | | | CTGGTACGTGGACGGCGTGGAGGTGCATAATGCCAAGA |
| | | | CAAAGCCGCGGGAGGAGCAGTACAACAGCACGTACCGT |
| | | | GTGGTCAGCGTCCTCACCGTCCTGCACCAGGACTGGCTG |
| | | | AATGGCAAGGAGTACAAGTGCAAGGTCTCCAACAAAGC |
| | | | CCTCGGCGCCCCCATCGAGAAAACCATCTCCAAAGCCAA |
| | | | AGGGCAGCCCCGAGAACCACAGGTGTACACCCTGCCCC |
| | | | CATCCCGGGATGAGCTGACCAAGAACCAGGTCAGCCTG |
| | | | ACCTGCCTGGTCAAAGGCTTCTATCCCAGCGACATCGCC |
| | | | GTGGAGTGGGAGAGCAATGGGCAGCCGGAGAACAACTA |
| | | | CAAGACCACGCCTCCCGTGCTGGACTCCGACGGCTCCTT |
| | | | CTTCCTCTACAGCAAGCTCACCGTGGACAAGAGCAGGTG |
| | | | GCAGCAGGGGAACGTCTTCTCATGCTCCGTGATGCATGA |
| | | | GGCTCTGCACAACCACTACACGCAGAAaAGCCTCTCCCT |
| | | | GTCTCCGGGTAAA |
| 26 | FGF- | Human | ACTCAGGGGGAGAATCACCCGTCTCCTAATTTTAACCAG |
| | 17R204Q | FGF-17 | TACGTGAGGGACCAGGGCGCCATGACCGACCAGCTGAG |
| | K207Q- | R204Q | CAGGCGGCAGATCCGCGAGTACCAACTCTACAGCAGGA |
| | linker1- | K207Q | CCAGTGGCAAGCACGTGCAGGTCACCGGGCGTCGCATCT |
| | hFcm | mutant- | CCGCCACCGCCGAGGACGGCAACAAGTTTGCCAAGCTC |
| | | linker1- | ATAGTGGAGACGGACACGTTTGGCAGCCGGGTTCGCATC |
| | | hFcm | AAAGGGGCTGAGAGTGAGAAGTACATCTGTATGAACAA |
| | | nucleotide | GAGGGGCAAGCTCATCGGGAAGCCCAGCGGGAAGAGCA |
| | | sequence | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCaGCGGACCcAGCGCACACGGCGGCCCCAGCCCCT |
| | | | CACGGGATCGGGATCGGACAAAACTCACACATGCCCAC |
| | | | CGTGCCCAGCACCTGAAGCTGCCGGGGGACCGTCAGTCT |
| | | | TCCTCTTCCCCCCAAAACCCAAGGACACCCTCATGATCT |
| | | | CCCGGACCCCTGAGGTCACATGCGTGGTGGTGGACGTGA |
| | | | GCCACGAAGACCCTGAGGTCAAGTTCAACTGGTACGTG |
| | | | GACGGCGTGGAGGTGCATAATGCCAAGACAAAGCCGCG |
| | | | GGAGGAGCAGTACAACAGCACGTACCGTGTGGTCAGCG |
| | | | TCCTCACCGTCCTGCACCAGGACTGGCTGAATGGCAAGG |
| | | | AGTACAAGTGCAAGGTCTCCAACAAAGCCCTCGGCGCC |
| | | | CCCATCGAGAAAACCATCTCCAAAGCCAAAGGGCAGCC |
| | | | CCGAGAACCACAGGTGTACACCCTGCCCCCATCCCGGGA |
| | | | TGAGCTGACCAAGAACCAGGTCAGCCTGACCTGCCTGGT |
| | | | CAAAGGCTTCTATCCCAGCGACATCGCCGTGGAGTGGGA |
| | | | GAGCAATGGGCAGCCGGAGAACAACTACAAGACCACGC |
| | | | CTCCCGTGCTGGACTCCGACGGCTCCTTCTTCCTCTACAG |
| | | | CAAGCTCACCGTGGACAAGAGCAGGTGGCAGCAGGGGA |
| | | | ACGTCTTCTCATGCTCCGTGATGCATGAGGCTCTGCACA |
| | | | ACCACTACACGCAGAAaAGCCTCTCCCTGTCTCCGGGTA |
| | | | AA |
| 27 | FGF- | Human | ACTCAGGGGGAGAATCACCCGTCTCCTAATTTTAACCAG |
| | 17d197- | FGF17 | TACGTGAGGGACCAGGGCGCCATGACCGACCAGCTGAG |
| | 216- | AA197-216 | CAGGCGGCAGATCCGCGAGTACCAACTCTACAGCAGGA |
| | linker1- | deletion | CCAGTGGCAAGCACGTGCAGGTCACCGGGCGTCGCATCT |
| | hFcm | mutant- | CCGCCACCGCCGAGGACGGCAACAAGTTTGCCAAGCTC |
| | | linker1- | ATAGTGGAGACGGACACGTTTGGCAGCCGGGTTCGCATC |
| | | hFcm | AAAGGGGCTGAGAGTGAGAAGTACATCTGTATGAACAA |
| | | nucleotide | GAGGGGCAAGCTCATCGGGAAGCCCAGCGGGAAGAGCA |
| | | sequence | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGGGATCGGGATCGGACA |
| | | | AAACTCACACATGCCCACCGTGCCCAGCACCTGAAGCTG |
| | | | CCGGGGGACCGTCAGTCTTCCTCTTCCCCCCAAAACCCA |
| | | | AGGACACCCTCATGATCTCCCGGACCCCTGAGGTCACAT |
| | | | GCGTGGTGGTGGACGTGAGCCACGAAGACCCTGAGGTC |
| | | | AAGTTCAACTGGTACGTGGACGGCGTGGAGGTGCATAA |
| | | | TGCCAAGACAAAGCCGCGGGAGGAGCAGTACAACAGCA |
| | | | CGTACCGTGTGGTCAGCGTCCTCACCGTCCTGCACCAGG |
| | | | ACTGGCTGAATGGCAAGGAGTACAAGTGCAAGGTCTCC |
| | | | AACAAAGCCCTCGGCGCCCCCATCGAGAAAACCATCTCC |
| | | | AAAGCCAAAGGGCAGCCCCGAGAACCACAGGTGTACAC |
| | | | CCTGCCCCCATCCCGGGATGAGCTGACCAAGAACCAGGT |
| | | | CAGCCTGACCTGCCTGGTCAAAGGCTTCTATCCCAGCGA |
| | | | CATCGCCGTGGAGTGGGAGAGCAATGGGCAGCCGGAGA |
| | | | ACAACTACAAGACCACGCCTCCCGTGCTGGACTCCGACG |
| | | | GCTCCTTCTTCCTCTACAGCAAGCTCACCGTGGACAAGA |
| | | | GCAGGTGGCAGCAGGGGAACGTCTTCTCATGCTCCGTGA |
| | | | TGCATGAGGCTCTGCACAACCACTACACGCAGAAaAGCC |
| | | | TCTCCCTGTCTCCGGGTAAA |
| 28 | FGF- | Human | ACTCAGGGGGAGAATCACCCGTCTCCTAATTTTAACCAG |
| | 17K191A | FGF-17 | TACGTGAGGGACCAGGGCGCCATGACCGACCAGCTGAG |
| | K193AS200A- | K191A | CAGGCGGCAGATCCGCGAGTACCAACTCTACAGCAGGA |
| | linker1- | K193A | CCAGTGGCAAGCACGTGCAGGTCACCGGGCGTCGCATCT |
| | hFcm | S200A | CCGCCACCGCCGAGGACGGCAACAAGTTTGCCAAGCTC |
| | | mutant- | ATAGTGGAGACGGACACGTTTGGCAGCCGGGTTCGCATC |
| | | linker1- | AAAGGGGCTGAGAGTGAGAAGTACATCTGTATGAACAA |
| | | hFcm | GAGGGGCAAGCTCATCGGGAAGCCCAGCGGGAAGAGCA |
| | | nucleotide | AAGACTGCGTGTTCACGGAGATCGTGCTGGAGAACAAC |
| | | sequence | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGGctCAGGcaCAGTTCGAGTTTGTGGGCgCtGCCCCCA |
| | | | CCCGCCGGACCAAGCGCACACGGCGGCCCCAGCCCCTC |
| | | | ACGGGATCGGGATCGGACAAAACTCACACATGCCCACC |
| | | | GTGCCCAGCACCTGAAGCTGCCGGGGGACCGTCAGTCTT |
| | | | CCTCTTCCCCCCAAAACCCAAGGACACCCTCATGATCTC |
| | | | CCGGACCCCTGAGGTCACATGCGTGGTGGTGGACGTGA |
| | | | GCCACGAAGACCCTGAGGTCAAGTTCAACTGGTACGTG |
| | | | GACGGCGTGGAGGTGCATAATGCCAAGACAAAGCCGCG |
| | | | GGAGGAGCAGTACAACAGCACGTACCGTGTGGTCAGCG |
| | | | TCCTCACCGTCCTGCACCAGGACTGGCTGAATGGCAAGG |
| | | | AGTACAAGTGCAAGGTCTCCAACAAAGCCCTCGGCGCC |
| | | | CCCATCGAGAAAACCATCTCCAAAGCCAAAGGGCAGCC |
| | | | CCGAGAACCACAGGTGTACACCCTGCCCCCATCCCGGGA |
| | | | TGAGCTGACCAAGAACCAGGTCAGCCTGACCTGCCTGGT |
| | | | CAAAGGCTTCTATCCCAGCGACATCGCCGTGGAGTGGGA |
| | | | GAGCAATGGGCAGCCGGAGAACAACTACAAGACCACGC |
| | | | CTCCCGTGCTGGACTCCGACGGCTCCTTCTTCCTCTACAG |
| | | | CAAGCTCACCGTGGACAAGAGCAGGTGGCAGCAGGGGA |
| | | | ACGTCTTCTCATGCTCCGTGATGCATGAGGCTCTGCACA |
| | | | ACCACTACACGCAGAAaAGCCTCTCCCTGTCTCCGGGTA |
| | | | AA |
| 29 | FGF-17- | Full length | ACTCAGGGGGAGAATCACCCGTCTCCTAATTTTAACCAG |
| | linker2- | of human | TACGTGAGGGACCAGGGCGCCATGACCGACCAGCTGAG |
| | hFcm | FGF17 | CAGGCGGCAGATCCGCGAGTACCAACTCTACAGCAGGA |
| | | linker2- | CCAGTGGCAAGCACGTGCAGGTCACCGGGCGTCGCATCT |
| | | hFcm | CCGCCACCGCCGAGGACGGCAACAAGTTTGCCAAGCTC |
| | | nucleotide | ATAGTGGAGACGGACACGTTTGGCAGCCGGGTTCGCATC |
| | | sequence | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCGCCGGACCAAGCGCACACGGCGGCCCCAGCCCC |
| | | | TCACGGGATCTGGGAGCGCTGACAAAACTCACACATGC |
| | | | CCACCGTGCCCAGCACCTGAAGCTGCCGGGGGACCGTC |
| | | | AGTCTTCCTCTTCCCCCCAAAACCCAAGGACACCCTCAT |
| | | | GATCTCCCGGACCCCTGAGGTCACATGCGTGGTGGTGGA |
| | | | CGTGAGCCACGAAGACCCTGAGGTCAAGTTCAACTGGT |
| | | | ACGTGGACGGCGTGGAGGTGCATAATGCCAAGACAAAG |
| | | | CCGCGGGAGGAGCAGTACAACAGCACGTACCGTGTGGT |
| | | | CAGCGTCCTCACCGTCCTGCACCAGGACTGGCTGAATGG |
| | | | CAAGGAGTACAAGTGCAAGGTCTCCAACAAAGCCCTCG |
| | | | GCGCCCCCATCGAGAAAACCATCTCCAAAGCCAAAGGG |
| | | | CAGCCCCGAGAACCACAGGTGTACACCCTGCCCCCATCC |
| | | | CGGGATGAGCTGACCAAGAACCAGGTCAGCCTGACCTG |
| | | | CCTGGTCAAAGGCTTCTATCCCAGCGACATCGCCGTGGA |
| | | | GTGGGAGAGCAATGGGCAGCCGGAGAACAACTACAAGA |
| | | | CCACGCCTCCCGTGCTGGACTCCGACGGCTCCTTCTTCCT |
| | | | CTACAGCAAGCTCACCGTGGACAAGAGCAGGTGGCAGC |
| | | | AGGGGAACGTCTTCTCATGCTCCGTGATGCATGAGGCTC |
| | | | TGCACAACCACTACACGCAGAAaAGCCTCTCCCTGTCTC |
| | | | CGGGTAAA |
| 30 | FGF- | Human | ACTCAGGGGGAGAATCACCCGTCTCCTAATTTTAACCAG |
| | 17d204- | FGF17 | TACGTGAGGGACCAGGGCGCCATGACCGACCAGCTGAG |
| | 216 | AA204-216 | CAGGCGGCAGATCCGCGAGTACCAACTCTACAGCAGGA |
| | linker2- | deletion | CCAGTGGCAAGCACGTGCAGGTCACCGGGCGTCGCATCT |
| | hFcm | mutant | CCGCCACCGCCGAGGACGGCAACAAGTTTGCCAAGCTC |
| | | linker2- | ATAGTGGAGACGGACACGTTTGGCAGCCGGGTTCGCATC |
| | | hFcm | AAAGGGGCTGAGAGTGAGAAGTACATCTGTATGAACAA |
| | | nucleotide | GAGGGGCAAGCTCATCGGGAAGCCCAGCGGGAAGAGCA |
| | | sequence | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCGGATCTGGGAGCGCTGACAAAACTCACACATGCC |
| | | | CACCGTGCCCAGCACCTGAAGCTGCCGGGGGACCGTCA |
| | | | GTCTTCCTCTTCCCCCCAAAACCCAAGGACACCCTCATG |
| | | | ATCTCCCGGACCCCTGAGGTCACATGCGTGGTGGTGGAC |
| | | | GTGAGCCACGAAGACCCTGAGGTCAAGTTCAACTGGTA |
| | | | CGTGGACGGCGTGGAGGTGCATAATGCCAAGACAAAGC |
| | | | CGCGGGAGGAGCAGTACAACAGCACGTACCGTGTGGTC |
| | | | AGCGTCCTCACCGTCCTGCACCAGGACTGGCTGAATGGC |
| | | | AAGGAGTACAAGTGCAAGGTCTCCAACAAAGCCCTCGG |
| | | | CGCCCCCATCGAGAAAACCATCTCCAAAGCCAAAGGGC |
| | | | AGCCCCGAGAACCACAGGTGTACACCCTGCCCCCATCCC |
| | | | GGGATGAGCTGACCAAGAACCAGGTCAGCCTGACCTGC |
| | | | CTGGTCAAAGGCTTCTATCCCAGCGACATCGCCGTGGAG |
| | | | TGGGAGAGCAATGGGCAGCCGGAGAACAACTACAAGAC |
| | | | CACGCCTCCCGTGCTGGACTCCGACGGCTCCTTCTTCCTC |
| | | | TACAGCAAGCTCACCGTGGACAAGAGCAGGTGGCAGCA |
| | | | GGGGAACGTCTTCTCATGCTCCGTGATGCATGAGGCTCT |
| | | | GCACAACCACTACACGCAGAAaAGCCTCTCCCTGTCTCC |
| | | | GGGTAAA |
| 31 | FGF- | Human | ACTCAGGGGGAGAATCACCCGTCTCCTAATTTTAACCAG |
| | 17d181- | FGF17 | TACGTGAGGGACCAGGGCGCCATGACCGACCAGCTGAG |
| | 216 | AA181-216 | CAGGCGGCAGATCCGCGAGTACCAACTCTACAGCAGGA |
| | linker2- | deletion | CCAGTGGCAAGCACGTGCAGGTCACCGGGCGTCGCATCT |
| | hFcm | mutant | CCGCCACCGCCGAGGACGGCAACAAGTTTGCCAAGCTC |
| | | linker2- | ATAGTGGAGACGGACACGTTTGGCAGCCGGGTTCGCATC |
| | | hFcm | AAAGGGGCTGAGAGTGAGAAGTACATCTGTATGAACAA |
| | | nucleotide | GAGGGGCAAGCTCATCGGGAAGCCCAGCGGGAAGAGCA |
| | | sequence | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGATCTGGGAGCGCTGACAAAACTC |
| | | | ACACATGCCCACCGTGCCCAGCACCTGAAGCTGCCGGG |
| | | | GGACCGTCAGTCTTCCTCTTCCCCCCAAAACCCAAGGAC |
| | | | ACCCTCATGATCTCCCGGACCCCTGAGGTCACATGCGTG |
| | | | GTGGTGGACGTGAGCCACGAAGACCCTGAGGTCAAGTT |
| | | | CAACTGGTACGTGGACGGCGTGGAGGTGCATAATGCCA |
| | | | AGACAAAGCCGCGGGAGGAGCAGTACAACAGCACGTAC |
| | | | CGTGTGGTCAGCGTCCTCACCGTCCTGCACCAGGACTGG |
| | | | CTGAATGGCAAGGAGTACAAGTGCAAGGTCTCCAACAA |
| | | | AGCCCTCGGCGCCCCCATCGAGAAAACCATCTCCAAAGC |
| | | | CAAAGGGCAGCCCCGAGAACCACAGGTGTACACCCTGC |
| | | | CCCCATCCCGGGATGAGCTGACCAAGAACCAGGTCAGC |
| | | | CTGACCTGCCTGGTCAAAGGCTTCTATCCCAGCGACATC |
| | | | GCCGTGGAGTGGGAGAGCAATGGGCAGCCGGAGAACAA |
| | | | CTACAAGACCACGCCTCCCGTGCTGGACTCCGACGGCTC |
| | | | CTTCTTCCTCTACAGCAAGCTCACCGTGGACAAGAGCAG |
| | | | GTGGCAGCAGGGGAACGTCTTCTCATGCTCCGTGATGCA |
| | | | TGAGGCTCTGCACAACCACTACACGCAGAAaAGCCTCTC |
| | | | CCTGTCTCCGGGTAAA |
| 32 | FGF- | Human | ACTCAGGGGGAGAATCACCCGTCTCCTAATTTTAACCAG |
| | 17R204Q | FGF-17 | TACGTGAGGGACCAGGGCGCCATGACCGACCAGCTGAG |
| | K207Q | R204Q | CAGGCGGCAGATCCGCGAGTACCAACTCTACAGCAGGA |
| | linker2- | K207Q | CCAGTGGCAAGCACGTGCAGGTCACCGGGCGTCGCATCT |
| | hFcm | mutant | CCGCCACCGCCGAGGACGGCAACAAGTTTGCCAAGCTC |
| | | linker2- | ATAGTGGAGACGGACACGTTTGGCAGCCGGGTTCGCATC |
| | | hFcm | AAAGGGGCTGAGAGTGAGAAGTACATCTGTATGAACAA |
| | | nucloetide | GAGGGGCAAGCTCATCGGGAAGCCCAGCGGGAAGAGCA |
| | | sequence | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCaGCGGACCcAGCGCACACGGCGGCCCCAGCCCCT |
| | | | CACGGGATCTGGGAGCGCTGACAAAACTCACACATGCC |
| | | | CACCGTGCCCAGCACCTGAAGCTGCCGGGGGACCGTCA |
| | | | GTCTTCCTCTTCCCCCCAAAACCCAAGGACACCCTCATG |
| | | | ATCTCCCGGACCCCTGAGGTCACATGCGTGGTGGTGGAC |
| | | | GTGAGCCACGAAGACCCTGAGGTCAAGTTCAACTGGTA |
| | | | CGTGGACGGCGTGGAGGTGCATAATGCCAAGACAAAGC |
| | | | CGCGGGAGGAGCAGTACAACAGCACGTACCGTGTGGTC |
| | | | AGCGTCCTCACCGTCCTGCACCAGGACTGGCTGAATGGC |
| | | | AAGGAGTACAAGTGCAAGGTCTCCAACAAAGCCCTCGG |
| | | | CGCCCCCATCGAGAAAACCATCTCCAAAGCCAAAGGGC |
| | | | AGCCCCGAGAACCACAGGTGTACACCCTGCCCCCATCCC |
| | | | GGGATGAGCTGACCAAGAACCAGGTCAGCCTGACCTGC |
| | | | CTGGTCAAAGGCTTCTATCCCAGCGACATCGCCGTGGAG |
| | | | TGGGAGAGCAATGGGCAGCCGGAGAACAACTACAAGAC |
| | | | CACGCCTCCCGTGCTGGACTCCGACGGCTCCTTCTTCCTC |
| | | | TACAGCAAGCTCACCGTGGACAAGAGCAGGTGGCAGCA |
| | | | GGGGAACGTCTTCTCATGCTCCGTGATGCATGAGGCTCT |
| | | | GCACAACCACTACACGCAGAAaAGCCTCTCCCTGTCTCC |
| | | | GGGTAAA |
| 33 | FGF- | Human | ACTCAGGGGGAGAATCACCCGTCTCCTAATTTTAACCAG |
| | 17d197- | FGF17 | TACGTGAGGGACCAGGGCGCCATGACCGACCAGCTGAG |
| | 216 | AA197-216 | CAGGCGGCAGATCCGCGAGTACCAACTCTACAGCAGGA |
| | linker2- | deletion | CCAGTGGCAAGCACGTGCAGGTCACCGGGCGTCGCATCT |
| | hFcm | mutant | CCGCCACCGCCGAGGACGGCAACAAGTTTGCCAAGCTC |
| | | linker2- | ATAGTGGAGACGGACACGTTTGGCAGCCGGGTTCGCATC |
| | | hFcm | AAAGGGGCTGAGAGTGAGAAGTACATCTGTATGAACAA |
| | | nucleotide | GAGGGGCAAGCTCATCGGGAAGCCCAGCGGGAAGAGCA |
| | | sequence | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGGGATCTGGGAGCGCTG |
| | | | ACAAAACTCACACATGCCCACCGTGCCCAGCACCTGAA |
| | | | GCTGCCGGGGGACCGTCAGTCTTCCTCTTCCCCCCAAAA |
| | | | CCCAAGGACACCCTCATGATCTCCCGGACCCCTGAGGTC |
| | | | ACATGCGTGGTGGTGGACGTGAGCCACGAAGACCCTGA |
| | | | GGTCAAGTTCAACTGGTACGTGGACGGCGTGGAGGTGC |
| | | | ATAATGCCAAGACAAAGCCGCGGGAGGAGCAGTACAAC |
| | | | AGCACGTACCGTGTGGTCAGCGTCCTCACCGTCCTGCAC |
| | | | CAGGACTGGCTGAATGGCAAGGAGTACAAGTGCAAGGT |
| | | | CTCCAACAAAGCCCTCGGCGCCCCCATCGAGAAAACCAT |
| | | | CTCCAAAGCCAAAGGGCAGCCCCGAGAACCACAGGTGT |
| | | | ACACCCTGCCCCCATCCCGGGATGAGCTGACCAAGAACC |
| | | | AGGTCAGCCTGACCTGCCTGGTCAAAGGCTTCTATCCCA |
| | | | GCGACATCGCCGTGGAGTGGGAGAGCAATGGGCAGCCG |
| | | | GAGAACAACTACAAGACCACGCCTCCCGTGCTGGACTCC |
| | | | GACGGCTCCTTCTTCCTCTACAGCAAGCTCACCGTGGAC |
| | | | AAGAGCAGGTGGCAGCAGGGGAACGTCTTCTCATGCTC |
| | | | CGTGATGCATGAGGCTCTGCACAACCACTACACGCAGA |
| | | | AaAGCCTCTCCCTGTCTCCGGGTAAA |
| 34 | FGF- | Human | ACTCAGGGGGAGAATCACCCGTCTCCTAATTTTAACCAG |
| | 17K191A | FGF-17 | TACGTGAGGGACCAGGGCGCCATGACCGACCAGCTGAG |
| | K193AS200A | K191A | CAGGCGGCAGATCCGCGAGTACCAACTCTACAGCAGGA |
| | linker2- | K193A | CCAGTGGCAAGCACGTGCAGGTCACCGGGCGTCGCATCT |
| | hFcm | S200A | CCGCCACCGCCGAGGACGGCAACAAGTTTGCCAAGCTC |
| | | mutant | ATAGTGGAGACGGACACGTTTGGCAGCCGGGTTCGCATC |
| | | linker2- | AAAGGGGCTGAGAGTGAGAAGTACATCTGTATGAACAA |
| | | hFcm | GAGGGGCAAGCTCATCGGGAAGCCCAGCGGGAAGAGCA |
| | | nucleotide | AAGACTGCGTGTTCACGGAGATCGTGCTGGAGAACAAC |
| | | sequence | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGGCTCAGGCACAGTTCGAGTTTGTGGGCGCTGCCCC |
| | | | CACCCGCCGGACCAAGCGCACACGGCGGCCCCAGCCCC |
| | | | TCACGGGATCTGGGAGCGCTGACAAAACTCACACATGC |
| | | | CCACCGTGCCCAGCACCTGAAGCTGCCGGGGGACCGTC |
| | | | AGTCTTCCTCTTCCCCCCAAAACCCAAGGACACCCTCAT |
| | | | GATCTCCCGGACCCCTGAGGTCACATGCGTGGTGGTGGA |
| | | | CGTGAGCCACGAAGACCCTGAGGTCAAGTTCAACTGGT |
| | | | ACGTGGACGGCGTGGAGGTGCATAATGCCAAGACAAAG |
| | | | CCGCGGGAGGAGCAGTACAACAGCACGTACCGTGTGGT |
| | | | CAGCGTCCTCACCGTCCTGCACCAGGACTGGCTGAATGG |
| | | | CAAGGAGTACAAGTGCAAGGTCTCCAACAAAGCCCTCG |
| | | | GCGCCCCCATCGAGAAAACCATCTCCAAAGCCAAAGGG |
| | | | CAGCCCCGAGAACCACAGGTGTACACCCTGCCCCCATCC |
| | | | CGGGATGAGCTGACCAAGAACCAGGTCAGCCTGACCTG |
| | | | CCTGGTCAAAGGCTTCTATCCCAGCGACATCGCCGTGGA |
| | | | GTGGGAGAGCAATGGGCAGCCGGAGAACAACTACAAGA |
| | | | CCACGCCTCCCGTGCTGGACTCCGACGGCTCCTTCTTCCT |
| | | | CTACAGCAAGCTCACCGTGGACAAGAGCAGGTGGCAGC |
| | | | AGGGGAACGTCTTCTCATGCTCCGTGATGCATGAGGCTC |
| | | | TGCACAACCACTACACGCAGAAaAGCCTCTCCCTGTCTC |
| | | | CGGGTAAA |
| 35 | 6xHis- | His tagged | CACCATCACCATCACCATAGCGGCGATGCACACAAGAG |
| | HSA- | HSA fusion | TGAGGTTGCTCATCGGTTTAAAGATTTGGGAGAAGAAAA |
| | linker3- | FGF17 with | TTTCAAAGCCTTGGTGTTGATTGCCTTTGCTCAGTATCTT |
| | FGF17 | a long | CAGCAGTGTCCATTTGAAGATCATGTAAAATTAGTGAAT |
| | | linker | GAAGTAACTGAATTTGCAAAAACATGTGTTGCTGATGAG |
| | | nucleotide | TCAGCTGAAAATTGTGACAAATCACTTCATACCCTTTTT |
| | | sequence | GGAGACAAATTATGCACAGTTGCAACTCTTCGTGAAACC |
| | | | TATGGTGAAATGGCTGACTGCTGTGCAAAACAAGAACCT |
| | | | GAGAGAAATGAATGCTTCTTGCAACACAAAGATGACAA |
| | | | CCCAAACCTCCCCCGATTGGTGAGACCAGAGGTTGATGT |
| | | | GATGTGCACTGCTTTTCATGACAATGAAGAGACATTTTT |
| | | | GAAAAAATACTTATATGAAATTGCCAGAAGACATCCTTA |
| | | | CTTTTATGCCCCGGAACTCCTTTTCTTTGCTAAAAGGTAT |
| | | | AAAGCTGCTTTTACAGAATGTTGCCAAGCTGCTGATAAA |
| | | | GCTGCCTGCCTGTTGCCAAAGCTCGATGAACTTCGGGAT |
| | | | GAAGGGAAGGCTTCGTCTGCCAAACAGAGACTCAAGTG |
| | | | TGCCAGTCTCCAAAAATTTGGAGAAAGAGCTTTCAAAGC |
| | | | ATGGGCAGTAGCTCGCCTGAGCCAGAGATTTCCCAAAGC |
| | | | TGAGTTTGCAGAAGTTTCCAAGTTAGTGACAGATCTTAC |
| | | | CAAAGTCCACACGGAATGCTGCCATGGAGATCTGCTTGA |
| | | | ATGTGCTGATGACAGGGCGGACCTTGCCAAGTATATCTG |
| | | | TGAAAATCAAGATTCGATCTCCAGTAAACTGAAGGAAT |
| | | | GCTGTGAAAAACCTCTGTTGGAAAAATCCCACTGCATTG |
| | | | CCGAAGTGGAAAATGATGAGATGCCTGCTGACTTGCCTT |
| | | | CATTAGCTGCTGATTTTGTTGAAAGTAAGGATGTTTGCA |
| | | | AAAACTATGCTGAGGCAAAGGATGTCTTCCTGGGCATGT |
| | | | TTTTGTATGAATATGCAAGAAGGCATCCTGATTACTCTG |
| | | | TCGTGCTGCTGCTGAGACTTGCCAAGACATATGAAACCA |
| | | | CTCTAGAGAAGTGCTGTGCCGCTGCAGATCCTCATGAAT |
| | | | GCTATGCCAAAGTGTTCGATGAATTTAAACCTCTTGTGG |
| | | | AAGAGCCTCAGAATTTAATCAAACAAAATTGTGAGCTTT |
| | | | TTGAGCAGCTTGGAGAGTACAAATTCCAGAATGCGCTAT |
| | | | TAGTTCGTTACACCAAGAAAGTACCCCAAGTGTCAACTC |
| | | | CAACTCTTGTAGAGGTCTCAAGAAACCTAGGAAAAGTG |
| | | | GGCAGCAAATGTTGTAAACATCCTGAAGCAAAAAGAAT |
| | | | GCCCTGTGCAGAAGACTATCTATCCGTGGTCCTGAACCA |
| | | | GTTATGTGTGTTGCATGAGAAAACGCCAGTAAGTGACAG |
| | | | AGTCACCAAATGCTGCACAGAATCCTTGGTGAACAGGC |
| | | | GACCATGCTTTTCAGCTCTGGAAGTCGATGAAACATACG |
| | | | TTCCCAAAGAGTTTAATGCTGAAACATTCACCTTCCATG |
| | | | CAGATATATGCACACTTTCTGAGAAGGAGAGACAAATC |
| | | | AAGAAACAAACTGCACTTGTTGAGCTCGTGAAACACAA |
| | | | GCCCAAGGCAACAAAAGAGCAACTGAAAGCTGTTATGG |
| | | | ATGATTTCGCAGCTTTTGTAGAGAAGTGCTGCAAGGCTG |
| | | | ACGATAAGGAGACCTGCTTTGCCGAGGAGGGTAAAAAA |
| | | | CTTGTTGCTGCAAGTCAAGCTGCCTTAGGCTTAGGCGGA |
| | | | GGCGGTAGCGGAGGCGGTGGCTCCGGTGGCGGAGGGTC |
| | | | TACTCAGGGGGAGAATCACCCGTCTCCTAATTTTAACCA |
| | | | GTACGTGAGGGACCAGGGCGCCATGACCGACCAGCTGA |
| | | | GCAGGCGGCAGATCCGCGAGTACCAACTCTACAGCAGG |
| | | | ACCAGTGGCAAGCACGTGCAGGTCACCGGGCGTCGCAT |
| | | | CTCCGCCACCGCCGAGGACGGCAACAAGTTTGCCAAGCT |
| | | | CATAGTGGAGACGGACACGTTTGGCAGCCGGGTTCGCAT |
| | | | CAAAGGGGCTGAGAGTGAGAAGTACATCTGTATGAACA |
| | | | AGAGGGGCAAGCTCATCGGGAAGCCCAGCGGGAAGAGC |
| | | | AAAGACTGCGTGTTCACGGAGATCGTGCTGGAGAACAA |
| | | | CTATACGGCCTTCCAGAACGCCCGGCACGAGGGCTGGTT |
| | | | CATGGCCTTCACGCGGCAGGGGCGGCCCCGCCAGGCTTC |
| | | | CCGCAGCCGCCAGAACCAGCGCGAGGCCCACTTCATCA |
| | | | AGCGCCTCTACCAAGGCCAGCTGCCCTTCCCCAACCACG |
| | | | CCGAGAAGCAGAAGCAGTTCGAGTTTGTGGGCTCCGCCC |
| | | | CCACCCGCCGGACCAAGCGCACACGGCGGCCCCAGCCC |
| | | | CTCACG |
| 36 | FGF17- | Human | ACTCAGGGGGAGAATCACCCGTCTCCTAATTTTAACCAG |
| | linker3- | FGF17- | TACGTGAGGGACCAGGGCGCCATGACCGACCAGCTGAG |
| | hFc4 | linker3- | CAGGCGGCAGATCCGCGAGTACCAACTCTACAGCAGGA |
| | | hFc4 | CCAGTGGCAAGCACGTGCAGGTCACCGGGCGTCGCATCT |
| | | nucleotide | CCGCCACCGCCGAGGACGGCAACAAGTTTGCCAAGCTC |
| | | sequence | ATAGTGGAGACGGACACGTTTGGCAGCCGGGTTCGCATC |
| | | | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCGCCGGACCAAGCGCACACGGCGGCCCCAGCCCC |
| | | | TCACGGGCGGAGGCGGTAGCGGAGGCGGTGGCTCCGGT |
| | | | GGCGGAGGGTCTGAGTCCAAATATGGTCCCCCATGCCCA |
| | | | CCCTGCCCAGCACCTGAGTTCCTGGGGGGACCATCAGTC |
| | | | TTCCTGTTCCCCCCAAAACCCAAGGACACTCTCATGATC |
| | | | TCCCGGACCCCTGAGGTCACGTGCGTGGTGGTGGACGTG |
| | | | AGCCAGGAAGACCCCGAGGTCCAGTTCAACTGGTACGT |
| | | | GGATGGCGTGGAGGTGCATAATGCCAAGACAAAGCCGC |
| | | | GGGAGGAGCAGTTCAACAGCACGTACCGTGTGGTCAGC |
| | | | GTCCTCACCGTCCTGCACCAGGACTGGCTGAACGGCAAG |
| | | | GAGTACAAGTGCAAGGTCTCCAACAAAGGCCTCCCGTCC |
| | | | TCCATCGAGAAAACCATCTCCAAAGCCAAAGGGCAGCC |
| | | | CCGAGAGCCACAGGTGTACACCCTGCCCCCATCCCAGGA |
| | | | GGAGATGACCAAGAACCAGGTCAGCCTGACCTGCCTGG |
| | | | TCAAAGGCTTCTACCCCAGCGACATCGCCGTGGAGTGGG |
| | | | AGAGCAATGGGCAGCCGGAGAACAACTACAAGACCACG |
| | | | CCTCCCGTGCTGGACTCCGACGGCTCCTTCTTCCTCTACA |
| | | | GCAGGCTCACCGTGGACAAGAGCAGGTGGCAGGAGGGG |
| | | | AATGTCTTCTCATGCTCCGTGATGCATGAGGCTCTGCAC |
| | | | AACCACTACACACAGAAGAGCCTCTCCCTGTCTCTGGGT |
| | | | AAA |
| 37 | FGF17- | Human | ACTCAGGGGGAGAATCACCCGTCTCCTAATTTTAACCAG |
| | hFc4 | FGF17- | TACGTGAGGGACCAGGGCGCCATGACCGACCAGCTGAG |
| | | hFc4 | CAGGCGGCAGATCCGCGAGTACCAACTCTACAGCAGGA |
| | | nucleotide | CCAGTGGCAAGCACGTGCAGGTCACCGGGCGTCGCATCT |
| | | sequence | CCGCCACCGCCGAGGACGGCAACAAGTTTGCCAAGCTC |
| | | | ATAGTGGAGACGGACACGTTTGGCAGCCGGGTTCGCATC |
| | | | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCGCCGGACCAAGCGCACACGGCGGCCCCAGCCCC |
| | | | TCACGGAGTCCAAATATGGTCCCCCATGCCCACCCTGCC |
| | | | CAGCACCTGAGTTCCTGGGGGGACCATCAGTCTTCCTGT |
| | | | TCCCCCCAAAACCCAAGGACACTCTCATGATCTCCCGGA |
| | | | CCCCTGAGGTCACGTGCGTGGTGGTGGACGTGAGCCAG |
| | | | GAAGACCCCGAGGTCCAGTTCAACTGGTACGTGGATGG |
| | | | CGTGGAGGTGCATAATGCCAAGACAAAGCCGCGGGAGG |
| | | | AGCAGTTCAACAGCACGTACCGTGTGGTCAGCGTCCTCA |
| | | | CCGTCCTGCACCAGGACTGGCTGAACGGCAAGGAGTAC |
| | | | AAGTGCAAGGTCTCCAACAAAGGCCTCCCGTCCTCCATC |
| | | | GAGAAAACCATCTCCAAAGCCAAAGGGCAGCCCCGAGA |
| | | | GCCACAGGTGTACACCCTGCCCCCATCCCAGGAGGAGAT |
| | | | GACCAAGAACCAGGTCAGCCTGACCTGCCTGGTCAAAG |
| | | | GCTTCTACCCCAGCGACATCGCCGTGGAGTGGGAGAGC |
| | | | AATGGGCAGCCGGAGAACAACTACAAGACCACGCCTCC |
| | | | CGTGCTGGACTCCGACGGCTCCTTCTTCCTCTACAGCAG |
| | | | GCTCACCGTGGACAAGAGCAGGTGGCAGGAGGGGAATG |
| | | | TCTTCTCATGCTCCGTGATGCATGAGGCTCTGCACAACC |
| | | | ACTACACACAGAAGAGCCTCTCCCTGTCTCTGGGTAAA |
| 38 | FGF17 | hFc4- | GAGTCCAAATATGGTCCCCCATGCCCACCCTGCCCAGCA |
| | hFc4L- | linker3- | CCTGAGTTCCTGGGGGGACCATCAGTCTTCCTGTTCCCC |
| | | human | CCAAAACCCAAGGACACTCTCATGATCTCCCGGACCCCT |
| | | FGF17 | GAGGTCACGTGCGTGGTGGTGGACGTGAGCCAGGAAGA |
| | | nucleotide | CCCCGAGGTCCAGTTCAACTGGTACGTGGATGGCGTGGA |
| | | sequence | GGTGCATAATGCCAAGACAAAGCCGCGGGAGGAGCAGT |
| | | | TCAACAGCACGTACCGTGTGGTCAGCGTCCTCACCGTCC |
| | | | TGCACCAGGACTGGCTGAACGGCAAGGAGTACAAGTGC |
| | | | AAGGTCTCCAACAAAGGCCTCCCGTCCTCCATCGAGAAA |
| | | | ACCATCTCCAAAGCCAAAGGGCAGCCCCGAGAGCCACA |
| | | | GGTGTACACCCTGCCCCCATCCCAGGAGGAGATGACCA |
| | | | AGAACCAGGTCAGCCTGACCTGCCTGGTCAAAGGCTTCT |
| | | | ACCCCAGCGACATCGCCGTGGAGTGGGAGAGCAATGGG |
| | | | CAGCCGGAGAACAACTACAAGACCACGCCTCCCGTGCT |
| | | | GGACTCCGACGGCTCCTTCTTCCTCTACAGCAGGCTCAC |
| | | | CGTGGACAAGAGCAGGTGGCAGGAGGGGAATGTCTTCT |
| | | | CATGCTCCGTGATGCATGAGGCTCTGCACAACCACTACA |
| | | | CACAGAAGAGCCTCTCCCTGTCTCTGGGTAAAGGCGGAG |
| | | | GCGGTAGCGGAGGCGGTGGCTCCGGTGGCGGAGGGTCT |
| | | | ACTCAGGGGGAGAATCACCCGTCTCCTAATTTTAACCAG |
| | | | TACGTGAGGGACCAGGGCGCCATGACCGACCAGCTGAG |
| | | | CAGGCGGCAGATCCGCGAGTACCAACTCTACAGCAGGA |
| | | | CCAGTGGCAAGCACGTGCAGGTCACCGGGCGTCGCATCT |
| | | | CCGCCACCGCCGAGGACGGCAACAAGTTTGCCAAGCTC |
| | | | ATAGTGGAGACGGACACGTTTGGCAGCCGGGTTCGCATC |
| | | | AAAGGGGCTGAGAGTGAGAAGTACATCTGTATGAACAA |
| | | | GAGGGGCAAGCTCATCGGGAAGCCCAGCGGGAAGAGCA |
| | | | AAGACTGCGTGTTCACGGAGATCGTGCTGGAGAACAAC |
| | | | TATACGGCCTTCCAGAACGCCCGGCACGAGGGCTGGTTC |
| | | | ATGGCCTTCACGCGGCAGGGGCGGCCCCGCCAGGCTTCC |
| | | | CGCAGCCGCCAGAACCAGCGCGAGGCCCACTTCATCAA |
| | | | GCGCCTCTACCAAGGCCAGCTGCCCTTCCCCAACCACGC |
| | | | CGAGAAGCAGAAGCAGTTCGAGTTTGTGGGCTCCGCCCC |
| | | | CACCCGCCGGACCAAGCGCACACGGCGGCCCCAGCCCC |
| | | | TCACG |
| 39 | IGF2 | nucleotide | GCTTACCGCCCCAGTGAGACCCTGTGCGGCGGGGAGCTG |
| | | sequence | GTGGACACCCTCCAGTTCGTCTGTGGGGACCGCGGCTTC |
| | | | TACTTCAGCAGGCCCGCAAGCCGTGTGAGCCGTCGCAGC |
| | | | CGTGGCATCGTTGAGGAGTGCTGTTTCCGCAGCTGTGAC |
| | | | CTGGCCCTCCTGGAGACGTACTGTGCTACCCCCGCCAAG |
| | | | TCCGAG |
| 40 | hFcm | nucleotide | GCTTACCGCCCCAGTGAGACCCTGTGCGGCGGGGAGCTG |
| | IGF2- | sequence | GTGGACACCCTCCAGTTCGTCTGTGGGGACCGCGGCTTC |
| | linker1- | | TACTTCAGCAGGCCCGCAAGCCGTGTGAGCCGTCGCAGC |
| | | | CGTGGCATCGTTGAGGAGTGCTGTTTCCGCAGCTGTGAC |
| | | | CTGGCCCTCCTGGAGACGTACTGTGCTACCCCCGCCAAG |
| | | | TCCGAG |
| | | | GGATCGGGATCGGACAAAACTCACACATGCCCACCGTG |
| | | | CCCAGCACCTGAAGCTGCCGGGGGACCGTCAGTCTTCCT |
| | | | CTTCCCCCCAAAACCCAAGGACACCCTCATGATCTCCCG |
| | | | GACCCCTGAGGTCACATGCGTGGTGGTGGACGTGAGCC |
| | | | ACGAAGACCCTGAGGTCAAGTTCAACTGGTACGTGGAC |
| | | | GGCGTGGAGGTGCATAATGCCAAGACAAAGCCGCGGGA |
| | | | GGAGCAGTACAACAGCACGTACCGTGTGGTCAGCGTCCT |
| | | | CACCGTCCTGCACCAGGACTGGCTGAATGGCAAGGAGT |
| | | | ACAAGTGCAAGGTCTCCAACAAAGCCCTCGGCGCCCCC |
| | | | ATCGAGAAAACCATCTCCAAAGCCAAAGGGCAGCCCCG |
| | | | AGAACCACAGGTGTACACCCTGCCCCCATCCCGGGATGA |
| | | | GCTGACCAAGAACCAGGTCAGCCTGACCTGCCTGGTCAA |
| | | | AGGCTTCTATCCCAGCGACATCGCCGTGGAGTGGGAGA |
| | | | GCAATGGGCAGCCGGAGAACAACTACAAGACCACGCCT |
| | | | CCCGTGCTGGACTCCGACGGCTCCTTCTTCCTCTACAGC |
| | | | AAGCTCACCGTGGACAAGAGCAGGTGGCAGCAGGGGAA |
| | | | CGTCTTCTCATGCTCCGTGATGCATGAGGCTCTGCACAA |
| | | | CCACTACACGCAGAAaAGCCTCTCCCTGTCTCCGGGTAA |
| | | | A |
| 41 | IGF2- | nucleotide | GCTTACCGCCCCAGTGAGACCCTGTGCGGCGGGGAGCTG |
| | linker2- | sequence | GTGGACACCCTCCAGTTCGTCTGTGGGGACCGCGGCTTC |
| | hFcm | | TACTTCAGCAGGCCCGCAAGCCGTGTGAGCCGTCGCAGC |
| | | | CGTGGCATCGTTGAGGAGTGCTGTTTCCGCAGCTGTGAC |
| | | | CTGGCCCTCCTGGAGACGTACTGTGCTACCCCCGCCAAG |
| | | | TCCGAG |
| | | | GGATCTGGGAGCGCTGACAAAACTCACACATGCCCACC |
| | | | GTGCCCAGCACCTGAAGCTGCCGGGGGACCGTCAGTCTT |
| | | | CCTCTTCCCCCCAAAACCCAAGGACACCCTCATGATCTC |
| | | | CCGGACCCCTGAGGTCACATGCGTGGTGGTGGACGTGA |
| | | | GCCACGAAGACCCTGAGGTCAAGTTCAACTGGTACGTG |
| | | | GACGGCGTGGAGGTGCATAATGCCAAGACAAAGCCGCG |
| | | | GGAGGAGCAGTACAACAGCACGTACCGTGTGGTCAGCG |
| | | | TCCTCACCGTCCTGCACCAGGACTGGCTGAATGGCAAGG |
| | | | AGTACAAGTGCAAGGTCTCCAACAAAGCCCTCGGCGCC |
| | | | CCCATCGAGAAAACCATCTCCAAAGCCAAAGGGCAGCC |
| | | | CCGAGAACCACAGGTGTACACCCTGCCCCCATCCCGGGA |
| | | | TGAGCTGACCAAGAACCAGGTCAGCCTGACCTGCCTGGT |
| | | | CAAAGGCTTCTATCCCAGCGACATCGCCGTGGAGTGGGA |
| | | | GAGCAATGGGCAGCCGGAGAACAACTACAAGACCACGC |
| | | | CTCCCGTGCTGGACTCCGACGGCTCCTTCTTCCTCTACAG |
| | | | CAAGCTCACCGTGGACAAGAGCAGGTGGCAGCAGGGGA |
| | | | ACGTCTTCTCATGCTCCGTGATGCATGAGGCTCTGCACA |
| | | | ACCACTACACGCAGAAaAGCCTCTCCCTGTCTCCGGGTA |
| | | | AA |
| 42 | 6xHis- | His tagged | CACCATCACCATCACCATAGCGGCGATGCACACAAGAG |
| | HSA- | HSA fusion | TGAGGTTGCTCATCGGTTTAAAGATTTGGGAGAAGAAAA |
| | linker3- | IGF2 with a | TTTCAAAGCCTTGGTGTTGATTGCCTTTGCTCAGTATCTT |
| | IGF2 | long linker | CAGCAGTGTCCATTTGAAGATCATGTAAAATTAGTGAAT |
| | | nucleotide | GAAGTAACTGAATTTGCAAAAACATGTGTTGCTGATGAG |
| | | sequence | TCAGCTGAAAATTGTGACAAATCACTTCATACCCTTTTT |
| | | | GGAGACAAATTATGCACAGTTGCAACTCTTCGTGAAACC |
| | | | TATGGTGAAATGGCTGACTGCTGTGCAAAACAAGAACCT |
| | | | GAGAGAAATGAATGCTTCTTGCAACACAAAGATGACAA |
| | | | CCCAAACCTCCCCCGATTGGTGAGACCAGAGGTTGATGT |
| | | | GATGTGCACTGCTTTTCATGACAATGAAGAGACATTTTT |
| | | | GAAAAAATACTTATATGAAATTGCCAGAAGACATCCTTA |
| | | | CTTTTATGCCCCGGAACTCCTTTTCTTTGCTAAAAGGTAT |
| | | | AAAGCTGCTTTTACAGAATGTTGCCAAGCTGCTGATAAA |
| | | | GCTGCCTGCCTGTTGCCAAAGCTCGATGAACTTCGGGAT |
| | | | GAAGGGAAGGCTTCGTCTGCCAAACAGAGACTCAAGTG |
| | | | TGCCAGTCTCCAAAAATTTGGAGAAAGAGCTTTCAAAGC |
| | | | ATGGGCAGTAGCTCGCCTGAGCCAGAGATTTCCCAAAGC |
| | | | TGAGTTTGCAGAAGTTTCCAAGTTAGTGACAGATCTTAC |
| | | | CAAAGTCCACACGGAATGCTGCCATGGAGATCTGCTTGA |
| | | | ATGTGCTGATGACAGGGCGGACCTTGCCAAGTATATCTG |
| | | | TGAAAATCAAGATTCGATCTCCAGTAAACTGAAGGAAT |
| | | | GCTGTGAAAAACCTCTGTTGGAAAAATCCCACTGCATTG |
| | | | CCGAAGTGGAAAATGATGAGATGCCTGCTGACTTGCCTT |
| | | | CATTAGCTGCTGATTTTGTTGAAAGTAAGGATGTTTGCA |
| | | | AAAACTATGCTGAGGCAAAGGATGTCTTCCTGGGCATGT |
| | | | TTTTGTATGAATATGCAAGAAGGCATCCTGATTACTCTG |
| | | | TCGTGCTGCTGCTGAGACTTGCCAAGACATATGAAACCA |
| | | | CTCTAGAGAAGTGCTGTGCCGCTGCAGATCCTCATGAAT |
| | | | GCTATGCCAAAGTGTTCGATGAATTTAAACCTCTTGTGG |
| | | | AAGAGCCTCAGAATTTAATCAAACAAAATTGTGAGCTTT |
| | | | TTGAGCAGCTTGGAGAGTACAAATTCCAGAATGCGCTAT |
| | | | TAGTTCGTTACACCAAGAAAGTACCCCAAGTGTCAACTC |
| | | | CAACTCTTGTAGAGGTCTCAAGAAACCTAGGAAAAGTG |
| | | | GGCAGCAAATGTTGTAAACATCCTGAAGCAAAAAGAAT |
| | | | GCCCTGTGCAGAAGACTATCTATCCGTGGTCCTGAACCA |
| | | | GTTATGTGTGTTGCATGAGAAAACGCCAGTAAGTGACAG |
| | | | AGTCACCAAATGCTGCACAGAATCCTTGGTGAACAGGC |
| | | | GACCATGCTTTTCAGCTCTGGAAGTCGATGAAACATACG |
| | | | TTCCCAAAGAGTTTAATGCTGAAACATTCACCTTCCATG |
| | | | CAGATATATGCACACTTTCTGAGAAGGAGAGACAAATC |
| | | | AAGAAACAAACTGCACTTGTTGAGCTCGTGAAACACAA |
| | | | GCCCAAGGCAACAAAAGAGCAACTGAAAGCTGTTATGG |
| | | | ATGATTTCGCAGCTTTTGTAGAGAAGTGCTGCAAGGCTG |
| | | | ACGATAAGGAGACCTGCTTTGCCGAGGAGGGTAAAAAA |
| | | | CTTGTTGCTGCAAGTCAAGCTGCCTTAGGCTTAGGCGGA |
| | | | GGCGGTAGCGGAGGCGGTGGCTCCGGTGGCGGAGGGTC |
| | | | TGCTTACCGCCCCAGTGAGACCCTGTGCGGCGGGGAGCT |
| | | | GGTGGACACCCTCCAGTTCGTCTGTGGGGACCGCGGCTT |
| | | | CTACTTCAGCAGGCCCGCAAGCCGTGTGAGCCGTCGCAG |
| | | | CCGTGGCATCGTTGAGGAGTGCTGTTTCCGCAGCTGTGA |
| | | | CCTGGCCCTCCTGGAGACGTACTGTGCTACCCCCGCCAA |
| | | | GTCCGAG |
| 43 | 6xHis- | His tagged | CACCATCACCATCACCATAGCGGCGATGCACACAAGAG |
| | HSA- | HSA fusion | TGAGGTTGCTCATCGGTTTAAAGATTTGGGAGAAGAAAA |
| | linker3- | IGF2 R61A | TTTCAAAGCCTTGGTGTTGATTGCCTTTGCTCAGTATCTT |
| | IGF2R61A | mutant with | CAGCAGTGTCCATTTGAAGATCATGTAAAATTAGTGAAT |
| | | a long | GAAGTAACTGAATTTGCAAAAACATGTGTTGCTGATGAG |
| | | linker | TCAGCTGAAAATTGTGACAAATCACTTCATACCCTTTTT |
| | | nucleotide | GGAGACAAATTATGCACAGTTGCAACTCTTCGTGAAACC |
| | | sequence | TATGGTGAAATGGCTGACTGCTGTGCAAAACAAGAACCT |
| | | | GAGAGAAATGAATGCTTCTTGCAACACAAAGATGACAA |
| | | | CCCAAACCTCCCCCGATTGGTGAGACCAGAGGTTGATGT |
| | | | GATGTGCACTGCTTTTCATGACAATGAAGAGACATTTTT |
| | | | GAAAAAATACTTATATGAAATTGCCAGAAGACATCCTTA |
| | | | CTTTTATGCCCCGGAACTCCTTTTCTTTGCTAAAAGGTAT |
| | | | AAAGCTGCTTTTACAGAATGTTGCCAAGCTGCTGATAAA |
| | | | GCTGCCTGCCTGTTGCCAAAGCTCGATGAACTTCGGGAT |
| | | | GAAGGGAAGGCTTCGTCTGCCAAACAGAGACTCAAGTG |
| | | | TGCCAGTCTCCAAAAATTTGGAGAAAGAGCTTTCAAAGC |
| | | | ATGGGCAGTAGCTCGCCTGAGCCAGAGATTTCCCAAAGC |
| | | | TGAGTTTGCAGAAGTTTCCAAGTTAGTGACAGATCTTAC |
| | | | CAAAGTCCACACGGAATGCTGCCATGGAGATCTGCTTGA |
| | | | ATGTGCTGATGACAGGGCGGACCTTGCCAAGTATATCTG |
| | | | TGAAAATCAAGATTCGATCTCCAGTAAACTGAAGGAAT |
| | | | GCTGTGAAAAACCTCTGTTGGAAAAATCCCACTGCATTG |
| | | | CCGAAGTGGAAAATGATGAGATGCCTGCTGACTTGCCTT |
| | | | CATTAGCTGCTGATTTTGTTGAAAGTAAGGATGTTTGCA |
| | | | AAAACTATGCTGAGGCAAAGGATGTCTTCCTGGGCATGT |
| | | | TTTTGTATGAATATGCAAGAAGGCATCCTGATTACTCTG |
| | | | TCGTGCTGCTGCTGAGACTTGCCAAGACATATGAAACCA |
| | | | CTCTAGAGAAGTGCTGTGCCGCTGCAGATCCTCATGAAT |
| | | | GCTATGCCAAAGTGTTCGATGAATTTAAACCTCTTGTGG |
| | | | AAGAGCCTCAGAATTTAATCAAACAAAATTGTGAGCTTT |
| | | | TTGAGCAGCTTGGAGAGTACAAATTCCAGAATGCGCTAT |
| | | | TAGTTCGTTACACCAAGAAAGTACCCCAAGTGTCAACTC |
| | | | CAACTCTTGTAGAGGTCTCAAGAAACCTAGGAAAAGTG |
| | | | GGCAGCAAATGTTGTAAACATCCTGAAGCAAAAAGAAT |
| | | | GCCCTGTGCAGAAGACTATCTATCCGTGGTCCTGAACCA |
| | | | GTTATGTGTGTTGCATGAGAAAACGCCAGTAAGTGACAG |
| | | | AGTCACCAAATGCTGCACAGAATCCTTGGTGAACAGGC |
| | | | GACCATGCTTTTCAGCTCTGGAAGTCGATGAAACATACG |
| | | | TTCCCAAAGAGTTTAATGCTGAAACATTCACCTTCCATG |
| | | | CAGATATATGCACACTTTCTGAGAAGGAGAGACAAATC |
| | | | AAGAAACAAACTGCACTTGTTGAGCTCGTGAAACACAA |
| | | | GCCCAAGGCAACAAAAGAGCAACTGAAAGCTGTTATGG |
| | | | ATGATTTCGCAGCTTTTGTAGAGAAGTGCTGCAAGGCTG |
| | | | ACGATAAGGAGACCTGCTTTGCCGAGGAGGGTAAAAAA |
| | | | CTTGTTGCTGCAAGTCAAGCTGCCTTAGGCTTAGGCGGA |
| | | | GGCGGTAGCGGAGGCGGTGGCTCCGGTGGCGGAGGGTC |
| | | | TGCTTACCGCCCCAGTGAGACCCTGTGCGGCGGGGAGCT |
| | | | GGTGGACACCCTCCAGTTCGTCTGTGGGGACCGCGGCTT |
| | | | CTACTTCAGCAGGCCCGCAAGCCGTGTGAGCGcTCGCAG |
| | | | CCGTGGCATCGTTGAGGAGTGCTGTTTCCGCAGCTGTGA |
| | | | CCTGGCCCTCCTGGAGACGTACTGTGCTACCCCCGCCAA |
| | | | GTCCGAG |
| 44 | 6xHis- | His tagged | CACCATCACCATCACCATAGCGGCGATGCACACAAGAG |
| | HSA- | HSA fusion | TGAGGTTGCTCATCGGTTTAAAGATTTGGGAGAAGAAAA |
| | linker3- | IGF2 R61Q | TTTCAAAGCCTTGGTGTTGATTGCCTTTGCTCAGTATCTT |
| | IGF2R61Q | mutant with | CAGCAGTGTCCATTTGAAGATCATGTAAAATTAGTGAAT |
| | | a long | GAAGTAACTGAATTTGCAAAAACATGTGTTGCTGATGAG |
| | | linker | TCAGCTGAAAATTGTGACAAATCACTTCATACCCTTTTT |
| | | nucleotide | GGAGACAAATTATGCACAGTTGCAACTCTTCGTGAAACC |
| | | sequence | TATGGTGAAATGGCTGACTGCTGTGCAAAACAAGAACCT |
| | | | GAGAGAAATGAATGCTTCTTGCAACACAAAGATGACAA |
| | | | CCCAAACCTCCCCCGATTGGTGAGACCAGAGGTTGATGT |
| | | | GATGTGCACTGCTTTTCATGACAATGAAGAGACATTTTT |
| | | | GAAAAAATACTTATATGAAATTGCCAGAAGACATCCTTA |
| | | | CTTTTATGCCCCGGAACTCCTTTTCTTTGCTAAAAGGTAT |
| | | | AAAGCTGCTTTTACAGAATGTTGCCAAGCTGCTGATAAA |
| | | | GCTGCCTGCCTGTTGCCAAAGCTCGATGAACTTCGGGAT |
| | | | GAAGGGAAGGCTTCGTCTGCCAAACAGAGACTCAAGTG |
| | | | TGCCAGTCTCCAAAAATTTGGAGAAAGAGCTTTCAAAGC |
| | | | ATGGGCAGTAGCTCGCCTGAGCCAGAGATTTCCCAAAGC |
| | | | TGAGTTTGCAGAAGTTTCCAAGTTAGTGACAGATCTTAC |
| | | | CAAAGTCCACACGGAATGCTGCCATGGAGATCTGCTTGA |
| | | | ATGTGCTGATGACAGGGCGGACCTTGCCAAGTATATCTG |
| | | | TGAAAATCAAGATTCGATCTCCAGTAAACTGAAGGAAT |
| | | | GCTGTGAAAAACCTCTGTTGGAAAAATCCCACTGCATTG |
| | | | CCGAAGTGGAAAATGATGAGATGCCTGCTGACTTGCCTT |
| | | | CATTAGCTGCTGATTTTGTTGAAAGTAAGGATGTTTGCA |
| | | | AAAACTATGCTGAGGCAAAGGATGTCTTCCTGGGCATGT |
| | | | TTTTGTATGAATATGCAAGAAGGCATCCTGATTACTCTG |
| | | | TCGTGCTGCTGCTGAGACTTGCCAAGACATATGAAACCA |
| | | | CTCTAGAGAAGTGCTGTGCCGCTGCAGATCCTCATGAAT |
| | | | GCTATGCCAAAGTGTTCGATGAATTTAAACCTCTTGTGG |
| | | | AAGAGCCTCAGAATTTAATCAAACAAAATTGTGAGCTTT |
| | | | TTGAGCAGCTTGGAGAGTACAAATTCCAGAATGCGCTAT |
| | | | TAGTTCGTTACACCAAGAAAGTACCCCAAGTGTCAACTC |
| | | | CAACTCTTGTAGAGGTCTCAAGAAACCTAGGAAAAGTG |
| | | | GGCAGCAAATGTTGTAAACATCCTGAAGCAAAAAGAAT |
| | | | GCCCTGTGCAGAAGACTATCTATCCGTGGTCCTGAACCA |
| | | | GTTATGTGTGTTGCATGAGAAAACGCCAGTAAGTGACAG |
| | | | AGTCACCAAATGCTGCACAGAATCCTTGGTGAACAGGC |
| | | | GACCATGCTTTTCAGCTCTGGAAGTCGATGAAACATACG |
| | | | TTCCCAAAGAGTTTAATGCTGAAACATTCACCTTCCATG |
| | | | CAGATATATGCACACTTTCTGAGAAGGAGAGACAAATC |
| | | | AAGAAACAAACTGCACTTGTTGAGCTCGTGAAACACAA |
| | | | GCCCAAGGCAACAAAAGAGCAACTGAAAGCTGTTATGG |
| | | | ATGATTTCGCAGCTTTTGTAGAGAAGTGCTGCAAGGCTG |
| | | | ACGATAAGGAGACCTGCTTTGCCGAGGAGGGTAAAAAA |
| | | | CTTGTTGCTGCAAGTCAAGCTGCCTTAGGCTTAGGCGGA |
| | | | GGCGGTAGCGGAGGCGGTGGCTCCGGTGGCGGAGGGTC |
| | | | TGCTTACCGCCCCAGTGAGACCCTGTGCGGCGGGGAGCT |
| | | | GGTGGACACCCTCCAGTTCGTCTGTGGGGACCGCGGCTT |
| | | | CTACTTCAGCAGGCCCGCAAGCCGTGTGAGCCaGCGCAG |
| | | | CCGTGGCATCGTTGAGGAGTGCTGTTTCCGCAGCTGTGA |
| | | | CCTGGCCCTCCTGGAGACGTACTGTGCTACCCCCGCCAA |
| | | | GTCCGAG |
| 45 | 6xHis- | His tagged | CACCATCACCATCACCATAGCGGCGATGCACACAAGAG |
| | HSA- | HSA fusion | TGAGGTTGCTCATCGGTTTAAAGATTTGGGAGAAGAAAA |
| | linker3- | IGF2 R64A | TTTCAAAGCCTTGGTGTTGATTGCCTTTGCTCAGTATCTT |
| | IGF2R64A | mutant with | CAGCAGTGTCCATTTGAAGATCATGTAAAATTAGTGAAT |
| | | a long | GAAGTAACTGAATTTGCAAAAACATGTGTTGCTGATGAG |
| | | linker | TCAGCTGAAAATTGTGACAAATCACTTCATACCCTTTTT |
| | | nucleotide | GGAGACAAATTATGCACAGTTGCAACTCTTCGTGAAACC |
| | | sequence | TATGGTGAAATGGCTGACTGCTGTGCAAAACAAGAACCT |
| | | | GAGAGAAATGAATGCTTCTTGCAACACAAAGATGACAA |
| | | | CCCAAACCTCCCCCGATTGGTGAGACCAGAGGTTGATGT |
| | | | GATGTGCACTGCTTTTCATGACAATGAAGAGACATTTTT |
| | | | GAAAAAATACTTATATGAAATTGCCAGAAGACATCCTTA |
| | | | CTTTTATGCCCCGGAACTCCTTTTCTTTGCTAAAAGGTAT |
| | | | AAAGCTGCTTTTACAGAATGTTGCCAAGCTGCTGATAAA |
| | | | GCTGCCTGCCTGTTGCCAAAGCTCGATGAACTTCGGGAT |
| | | | GAAGGGAAGGCTTCGTCTGCCAAACAGAGACTCAAGTG |
| | | | TGCCAGTCTCCAAAAATTTGGAGAAAGAGCTTTCAAAGC |
| | | | ATGGGCAGTAGCTCGCCTGAGCCAGAGATTTCCCAAAGC |
| | | | TGAGTTTGCAGAAGTTTCCAAGTTAGTGACAGATCTTAC |
| | | | CAAAGTCCACACGGAATGCTGCCATGGAGATCTGCTTGA |
| | | | ATGTGCTGATGACAGGGCGGACCTTGCCAAGTATATCTG |
| | | | TGAAAATCAAGATTCGATCTCCAGTAAACTGAAGGAAT |
| | | | GCTGTGAAAAACCTCTGTTGGAAAAATCCCACTGCATTG |
| | | | CCGAAGTGGAAAATGATGAGATGCCTGCTGACTTGCCTT |
| | | | CATTAGCTGCTGATTTTGTTGAAAGTAAGGATGTTTGCA |
| | | | AAAACTATGCTGAGGCAAAGGATGTCTTCCTGGGCATGT |
| | | | TTTTGTATGAATATGCAAGAAGGCATCCTGATTACTCTG |
| | | | TCGTGCTGCTGCTGAGACTTGCCAAGACATATGAAACCA |
| | | | CTCTAGAGAAGTGCTGTGCCGCTGCAGATCCTCATGAAT |
| | | | GCTATGCCAAAGTGTTCGATGAATTTAAACCTCTTGTGG |
| | | | AAGAGCCTCAGAATTTAATCAAACAAAATTGTGAGCTTT |
| | | | TTGAGCAGCTTGGAGAGTACAAATTCCAGAATGCGCTAT |
| | | | TAGTTCGTTACACCAAGAAAGTACCCCAAGTGTCAACTC |
| | | | CAACTCTTGTAGAGGTCTCAAGAAACCTAGGAAAAGTG |
| | | | GGCAGCAAATGTTGTAAACATCCTGAAGCAAAAAGAAT |
| | | | GCCCTGTGCAGAAGACTATCTATCCGTGGTCCTGAACCA |
| | | | GTTATGTGTGTTGCATGAGAAAACGCCAGTAAGTGACAG |
| | | | AGTCACCAAATGCTGCACAGAATCCTTGGTGAACAGGC |
| | | | GACCATGCTTTTCAGCTCTGGAAGTCGATGAAACATACG |
| | | | TTCCCAAAGAGTTTAATGCTGAAACATTCACCTTCCATG |
| | | | CAGATATATGCACACTTTCTGAGAAGGAGAGACAAATC |
| | | | AAGAAACAAACTGCACTTGTTGAGCTCGTGAAACACAA |
| | | | GCCCAAGGCAACAAAAGAGCAACTGAAAGCTGTTATGG |
| | | | ATGATTTCGCAGCTTTTGTAGAGAAGTGCTGCAAGGCTG |
| | | | ACGATAAGGAGACCTGCTTTGCCGAGGAGGGTAAAAAA |
| | | | CTTGTTGCTGCAAGTCAAGCTGCCTTAGGCTTAGGCGGA |
| | | | GGCGGTAGCGGAGGCGGTGGCTCCGGTGGCGGAGGGTC |
| | | | TGCTTACCGCCCCAGTGAGACCCTGTGCGGCGGGGAGCT |
| | | | GGTGGACACCCTCCAGTTCGTCTGTGGGGACCGCGGCTT |
| | | | CTACTTCAGCAGGCCCGCAAGCCGTGTGAGCCGTCGCAG |
| | | | CGCTGGCATCGTTGAGGAGTGCTGTTTCCGCAGCTGTGA |
| | | | CCTGGCCCTCCTGGAGACGTACTGTGCTACCCCCGCCAA |
| | | | GTCCGAG |
| 46 | 6xHis- | His tagged | CACCATCACCATCACCATAGCGGCGATGCACACAAGAG |
| | HSA- | HSA fusion | TGAGGTTGCTCATCGGTTTAAAGATTTGGGAGAAGAAAA |
| | linker3- | IGF2 R64Q | TTTCAAAGCCTTGGTGTTGATTGCCTTTGCTCAGTATCTT |
| | IGF2R64Q | mutant with | CAGCAGTGTCCATTTGAAGATCATGTAAAATTAGTGAAT |
| | | a long | GAAGTAACTGAATTTGCAAAAACATGTGTTGCTGATGAG |
| | | linker | TCAGCTGAAAATTGTGACAAATCACTTCATACCCTTTTT |
| | | nucleotide | GGAGACAAATTATGCACAGTTGCAACTCTTCGTGAAACC |
| | | sequence | TATGGTGAAATGGCTGACTGCTGTGCAAAACAAGAACCT |
| | | | GAGAGAAATGAATGCTTCTTGCAACACAAAGATGACAA |
| | | | CCCAAACCTCCCCCGATTGGTGAGACCAGAGGTTGATGT |
| | | | GATGTGCACTGCTTTTCATGACAATGAAGAGACATTTTT |
| | | | GAAAAAATACTTATATGAAATTGCCAGAAGACATCCTTA |
| | | | CTTTTATGCCCCGGAACTCCTTTTCTTTGCTAAAAGGTAT |
| | | | AAAGCTGCTTTTACAGAATGTTGCCAAGCTGCTGATAAA |
| | | | GCTGCCTGCCTGTTGCCAAAGCTCGATGAACTTCGGGAT |
| | | | GAAGGGAAGGCTTCGTCTGCCAAACAGAGACTCAAGTG |
| | | | TGCCAGTCTCCAAAAATTTGGAGAAAGAGCTTTCAAAGC |
| | | | ATGGGCAGTAGCTCGCCTGAGCCAGAGATTTCCCAAAGC |
| | | | TGAGTTTGCAGAAGTTTCCAAGTTAGTGACAGATCTTAC |
| | | | CAAAGTCCACACGGAATGCTGCCATGGAGATCTGCTTGA |
| | | | ATGTGCTGATGACAGGGCGGACCTTGCCAAGTATATCTG |
| | | | TGAAAATCAAGATTCGATCTCCAGTAAACTGAAGGAAT |
| | | | GCTGTGAAAAACCTCTGTTGGAAAAATCCCACTGCATTG |
| | | | CCGAAGTGGAAAATGATGAGATGCCTGCTGACTTGCCTT |
| | | | CATTAGCTGCTGATTTTGTTGAAAGTAAGGATGTTTGCA |
| | | | AAAACTATGCTGAGGCAAAGGATGTCTTCCTGGGCATGT |
| | | | TTTTGTATGAATATGCAAGAAGGCATCCTGATTACTCTG |
| | | | TCGTGCTGCTGCTGAGACTTGCCAAGACATATGAAACCA |
| | | | CTCTAGAGAAGTGCTGTGCCGCTGCAGATCCTCATGAAT |
| | | | GCTATGCCAAAGTGTTCGATGAATTTAAACCTCTTGTGG |
| | | | AAGAGCCTCAGAATTTAATCAAACAAAATTGTGAGCTTT |
| | | | TTGAGCAGCTTGGAGAGTACAAATTCCAGAATGCGCTAT |
| | | | TAGTTCGTTACACCAAGAAAGTACCCCAAGTGTCAACTC |
| | | | CAACTCTTGTAGAGGTCTCAAGAAACCTAGGAAAAGTG |
| | | | GGCAGCAAATGTTGTAAACATCCTGAAGCAAAAAGAAT |
| | | | GCCCTGTGCAGAAGACTATCTATCCGTGGTCCTGAACCA |
| | | | GTTATGTGTGTTGCATGAGAAAACGCCAGTAAGTGACAG |
| | | | AGTCACCAAATGCTGCACAGAATCCTTGGTGAACAGGC |
| | | | GACCATGCTTTTCAGCTCTGGAAGTCGATGAAACATACG |
| | | | TTCCCAAAGAGTTTAATGCTGAAACATTCACCTTCCATG |
| | | | CAGATATATGCACACTTTCTGAGAAGGAGAGACAAATC |
| | | | AAGAAACAAACTGCACTTGTTGAGCTCGTGAAACACAA |
| | | | GCCCAAGGCAACAAAAGAGCAACTGAAAGCTGTTATGG |
| | | | ATGATTTCGCAGCTTTTGTAGAGAAGTGCTGCAAGGCTG |
| | | | ACGATAAGGAGACCTGCTTTGCCGAGGAGGGTAAAAAA |
| | | | CTTGTTGCTGCAAGTCAAGCTGCCTTAGGCTTAGGCGGA |
| | | | GGCGGTAGCGGAGGCGGTGGCTCCGGTGGCGGAGGGTC |
| | | | TGCTTACCGCCCCAGTGAGACCCTGTGCGGCGGGGAGCT |
| | | | GGTGGACACCCTCCAGTTCGTCTGTGGGGACCGCGGCTT |
| | | | CTACTTCAGCAGGCCCGCAAGCCGTGTGAGCCGTCGCAG |
| | | | CCaGGGCATCGTTGAGGAGTGCTGTTTCCGCAGCTGTGA |
| | | | CCTGGCCCTCCTGGAGACGTACTGTGCTACCCCCGCCAA |
| | | | GTCCGAG |
| 47 | IGF2- | Human | GCTTACCGCCCCAGTGAGACCCTGTGCGGCGGGGAGCTG |
| | linker3- | IGF2- | GTGGACACCCTCCAGTTCGTCTGTGGGGACCGCGGCTTC |
| | hFc4 | linker3- | TACTTCAGCAGGCCCGCAAGCCGTGTGAGCCGTCGCAGC |
| | | hFc4 | CGTGGCATCGTTGAGGAGTGCTGTTTCCGCAGCTGTGAC |
| | | nucleotide | CTGGCCCTCCTGGAGACGTACTGTGCTACCCCCGCCAAG |
| | | sequence | TCCGAGGGCGGAGGCGGTAGCGGAGGCGGTGGCTCCGG |
| | | | TGGCGGAGGGTCTGAGTCCAAATATGGTCCCCCATGCCC |
| | | | ACCCTGCCCAGCACCTGAGTTCCTGGGGGGACCATCAGT |
| | | | CTTCCTGTTCCCCCCAAAACCCAAGGACACTCTCATGAT |
| | | | CTCCCGGACCCCTGAGGTCACGTGCGTGGTGGTGGACGT |
| | | | GAGCCAGGAAGACCCCGAGGTCCAGTTCAACTGGTACG |
| | | | TGGATGGCGTGGAGGTGCATAATGCCAAGACAAAGCCG |
| | | | CGGGAGGAGCAGTTCAACAGCACGTACCGTGTGGTCAG |
| | | | CGTCCTCACCGTCCTGCACCAGGACTGGCTGAACGGCAA |
| | | | GGAGTACAAGTGCAAGGTCTCCAACAAAGGCCTCCCGT |
| | | | CCTCCATCGAGAAAACCATCTCCAAAGCCAAAGGGCAG |
| | | | CCCCGAGAGCCACAGGTGTACACCCTGCCCCCATCCCAG |
| | | | GAGGAGATGACCAAGAACCAGGTCAGCCTGACCTGCCT |
| | | | GGTCAAAGGCTTCTACCCCAGCGACATCGCCGTGGAGTG |
| | | | GGAGAGCAATGGGCAGCCGGAGAACAACTACAAGACCA |
| | | | CGCCTCCCGTGCTGGACTCCGACGGCTCCTTCTTCCTCTA |
| | | | CAGCAGGCTCACCGTGGACAAGAGCAGGTGGCAGGAGG |
| | | | GGAATGTCTTCTCATGCTCCGTGATGCATGAGGCTCTGC |
| | | | ACAACCACTACACACAGAAGAGCCTCTCCCTGTCTCTGG |
| | | | GTAAA |
| 48 | IGF2- | Human | GCTTACCGCCCCAGTGAGACCCTGTGCGGCGGGGAGCTG |
| | hFc4 | IGF2-hFc4 | GTGGACACCCTCCAGTTCGTCTGTGGGGACCGCGGCTTC |
| | | nucleotide | TACTTCAGCAGGCCCGCAAGCCGTGTGAGCCGTCGCAGC |
| | | sequence | CGTGGCATCGTTGAGGAGTGCTGTTTCCGCAGCTGTGAC |
| | | | CTGGCCCTCCTGGAGACGTACTGTGCTACCCCCGCCAAG |
| | | | TCCGAGGAGTCCAAATATGGTCCCCCATGCCCACCCTGC |
| | | | CCAGCACCTGAGTTCCTGGGGGGACCATCAGTCTTCCTG |
| | | | TTCCCCCCAAAACCCAAGGACACTCTCATGATCTCCCGG |
| | | | ACCCCTGAGGTCACGTGCGTGGTGGTGGACGTGAGCCA |
| | | | GGAAGACCCCGAGGTCCAGTTCAACTGGTACGTGGATG |
| | | | GCGTGGAGGTGCATAATGCCAAGACAAAGCCGCGGGAG |
| | | | GAGCAGTTCAACAGCACGTACCGTGTGGTCAGCGTCCTC |
| | | | ACCGTCCTGCACCAGGACTGGCTGAACGGCAAGGAGTA |
| | | | CAAGTGCAAGGTCTCCAACAAAGGCCTCCCGTCCTCCAT |
| | | | CGAGAAAACCATCTCCAAAGCCAAAGGGCAGCCCCGAG |
| | | | AGCCACAGGTGTACACCCTGCCCCCATCCCAGGAGGAG |
| | | | ATGACCAAGAACCAGGTCAGCCTGACCTGCCTGGTCAA |
| | | | AGGCTTCTACCCCAGCGACATCGCCGTGGAGTGGGAGA |
| | | | GCAATGGGCAGCCGGAGAACAACTACAAGACCACGCCT |
| | | | CCCGTGCTGGACTCCGACGGCTCCTTCTTCCTCTACAGC |
| | | | AGGCTCACCGTGGACAAGAGCAGGTGGCAGGAGGGGAA |
| | | | TGTCTTCTCATGCTCCGTGATGCATGAGGCTCTGCACAA |
| | | | CCACTACACACAGAAGAGCCTCTCCCTGTCTCTGGGTAA |
| | | | A |
| 49 | hFc4- | hFc4- | GAGTCCAAATATGGTCCCCCATGCCCACCCTGCCCAGCA |
| | linker3- | linker3- | CCTGAGTTCCTGGGGGGACCATCAGTCTTCCTGTTCCCC |
| | IGF2 | human | CCAAAACCCAAGGACACTCTCATGATCTCCCGGACCCCT |
| | | IGF2 | GAGGTCACGTGCGTGGTGGTGGACGTGAGCCAGGAAGA |
| | | nucleotide | CCCCGAGGTCCAGTTCAACTGGTACGTGGATGGCGTGGA |
| | | sequence | GGTGCATAATGCCAAGACAAAGCCGCGGGAGGAGCAGT |
| | | | TCAACAGCACGTACCGTGTGGTCAGCGTCCTCACCGTCC |
| | | | TGCACCAGGACTGGCTGAACGGCAAGGAGTACAAGTGC |
| | | | AAGGTCTCCAACAAAGGCCTCCCGTCCTCCATCGAGAAA |
| | | | ACCATCTCCAAAGCCAAAGGGCAGCCCCGAGAGCCACA |
| | | | GGTGTACACCCTGCCCCCATCCCAGGAGGAGATGACCA |
| | | | AGAACCAGGTCAGCCTGACCTGCCTGGTCAAAGGCTTCT |
| | | | ACCCCAGCGACATCGCCGTGGAGTGGGAGAGCAATGGG |
| | | | CAGCCGGAGAACAACTACAAGACCACGCCTCCCGTGCT |
| | | | GGACTCCGACGGCTCCTTCTTCCTCTACAGCAGGCTCAC |
| | | | CGTGGACAAGAGCAGGTGGCAGGAGGGGAATGTCTTCT |
| | | | CATGCTCCGTGATGCATGAGGCTCTGCACAACCACTACA |
| | | | CACAGAAGAGCCTCTCCCTGTCTCTGGGTAAAGGCGGAG |
| | | | GCGGTAGCGGAGGCGGTGGCTCCGGTGGCGGAGGGTCT |
| | | | GCTTACCGCCCCAGTGAGACCCTGTGCGGCGGGGAGCTG |
| | | | GTGGACACCCTCCAGTTCGTCTGTGGGGACCGCGGCTTC |
| | | | TACTTCAGCAGGCCCGCAAGCCGTGTGAGCCGTCGCAGC |
| | | | CGTGGCATCGTTGAGGAGTGCTGTTTCCGCAGCTGTGAC |
| | | | CTGGCCCTCCTGGAGACGTACTGTGCTACCCCCGCCAAG |
| | | | TCCGAG |
| 50 | IGF2R61A- | Human | GCTTACCGCCCCAGTGAGACCCTGTGCGGCGGGGAGCTG |
| | linker3- | IGF2 R61A | GTGGACACCCTCCAGTTCGTCTGTGGGGACCGCGGCTTC |
| | hFc4 | point | TACTTCAGCAGGCCCGCAAGCCGTGTGAGCGcTCGCAGC |
| | | mutant- | CGTGGCATCGTTGAGGAGTGCTGTTTCCGCAGCTGTGAC |
| | | linker3- | CTGGCCCTCCTGGAGACGTACTGTGCTACCCCCGCCAAG |
| | | hFc4 | TCCGAGGGCGGAGGCGGTAGCGGAGGCGGTGGCTCCGG |
| | | nucleotide | TGGCGGAGGGTCTGAGTCCAAATATGGTCCCCCATGCCC |
| | | sequence | ACCCTGCCCAGCACCTGAGTTCCTGGGGGGACCATCAGT |
| | | | CTTCCTGTTCCCCCCAAAACCCAAGGACACTCTCATGAT |
| | | | CTCCCGGACCCCTGAGGTCACGTGCGTGGTGGTGGACGT |
| | | | GAGCCAGGAAGACCCCGAGGTCCAGTTCAACTGGTACG |
| | | | TGGATGGCGTGGAGGTGCATAATGCCAAGACAAAGCCG |
| | | | CGGGAGGAGCAGTTCAACAGCACGTACCGTGTGGTCAG |
| | | | CGTCCTCACCGTCCTGCACCAGGACTGGCTGAACGGCAA |
| | | | GGAGTACAAGTGCAAGGTCTCCAACAAAGGCCTCCCGT |
| | | | CCTCCATCGAGAAAACCATCTCCAAAGCCAAAGGGCAG |
| | | | CCCCGAGAGCCACAGGTGTACACCCTGCCCCCATCCCAG |
| | | | GAGGAGATGACCAAGAACCAGGTCAGCCTGACCTGCCT |
| | | | GGTCAAAGGCTTCTACCCCAGCGACATCGCCGTGGAGTG |
| | | | GGAGAGCAATGGGCAGCCGGAGAACAACTACAAGACCA |
| | | | CGCCTCCCGTGCTGGACTCCGACGGCTCCTTCTTCCTCTA |
| | | | CAGCAGGCTCACCGTGGACAAGAGCAGGTGGCAGGAGG |
| | | | GGAATGTCTTCTCATGCTCCGTGATGCATGAGGCTCTGC |
| | | | ACAACCACTACACACAGAAGAGCCTCTCCCTGTCTCTGG |
| | | | GTAAA |
| 51 | BMP-7 | Mature | TCCACGGGGAGCAAACAGCGCAGCCAGAACCGCTCCAA |
| | | form of | GACGCCCAAGAACCAGGAAGCCCTGCGGATGGCCAACG |
| | | human | TGGCAGAGAACAGCAGCAGCGACCAGAGGCAGGCCTGT |
| | | BMP-7 | AAGAAGCACGAGCTGTATGTCAGCTTCCGAGACCTGGG |
| | | (AA293- | CTGGCAGGACTGGATCATCGCGCCTGAAGGCTACGCCGC |
| | | 431) | CTACTACTGTGAGGGGGAGTGTGCCTTCCCTCTGAACTC |
| | | nucleotide | CTACATGAACGCCACCAACCACGCCATCGTGCAGACGCT |
| | | sequence | GGTCCACTTCATCAACCCGGAAACGGTGCCCAAGCCCTG |
| | | | CTGTGCGCCCACGCAGCTCAATGCCATCTCCGTCCTCTA |
| | | | CTTCGATGACAGCTCCAACGTCATCCTGAAGAAATACAG |
| | | | AAACATGGTGGTCCGGGCCTGTGGCTGCCAC |
| 52 | BMP-7- | BMP7 | TCCACGGGGAGCAAACAGCGCAGCCAGAACCGCTCCAA |
| | linker1- | fusion | GACGCCCAAGAACCAGGAAGCCCTGCGGATGGCCAACG |
| | hFcm | protein | TGGCAGAGAACAGCAGCAGCGACCAGAGGCAGGCCTGT |
| | | nucleotide | AAGAAGCACGAGCTGTATGTCAGCTTCCGAGACCTGGG |
| | | sequence | CTGGCAGGACTGGATCATCGCGCCTGAAGGCTACGCCGC |
| | | | CTACTACTGTGAGGGGGAGTGTGCCTTCCCTCTGAACTC |
| | | | CTACATGAACGCCACCAACCACGCCATCGTGCAGACGCT |
| | | | GGTCCACTTCATCAACCCGGAAACGGTGCCCAAGCCCTG |
| | | | CTGTGCGCCCACGCAGCTCAATGCCATCTCCGTCCTCTA |
| | | | CTTCGATGACAGCTCCAACGTCATCCTGAAGAAATACAG |
| | | | AAACATGGTGGTCCGGGCCTGTGGCTGCCAC |
| | | | GGATCGGGATCGGACAAAACTCACACATGCCCACCGTG |
| | | | CCCAGCACCTGAAGCTGCCGGGGGACCGTCAGTCTTCCT |
| | | | CTTCCCCCCAAAACCCAAGGACACCCTCATGATCTCCCG |
| | | | GACCCCTGAGGTCACATGCGTGGTGGTGGACGTGAGCC |
| | | | ACGAAGACCCTGAGGTCAAGTTCAACTGGTACGTGGAC |
| | | | GGCGTGGAGGTGCATAATGCCAAGACAAAGCCGCGGGA |
| | | | GGAGCAGTACAACAGCACGTACCGTGTGGTCAGCGTCCT |
| | | | CACCGTCCTGCACCAGGACTGGCTGAATGGCAAGGAGT |
| | | | ACAAGTGCAAGGTCTCCAACAAAGCCCTCGGCGCCCCC |
| | | | ATCGAGAAAACCATCTCCAAAGCCAAAGGGCAGCCCCG |
| | | | AGAACCACAGGTGTACACCCTGCCCCCATCCCGGGATGA |
| | | | GCTGACCAAGAACCAGGTCAGCCTGACCTGCCTGGTCAA |
| | | | AGGCTTCTATCCCAGCGACATCGCCGTGGAGTGGGAGA |
| | | | GCAATGGGCAGCCGGAGAACAACTACAAGACCACGCCT |
| | | | CCCGTGCTGGACTCCGACGGCTCCTTCTTCCTCTACAGC |
| | | | AAGCTCACCGTGGACAAGAGCAGGTGGCAGCAGGGGAA |
| | | | CGTCTTCTCATGCTCCGTGATGCATGAGGCTCTGCACAA |
| | | | CCACTACACGCAGAAaAGCCTCTCCCTGTCTCCGGGTAA |
| | | | A |
| 53 | BMP-7- | BMP7 | TCCACGGGGAGCAAACAGCGCAGCCAGAACCGCTCCAA |
| | linker2- | fusion | GACGCCCAAGAACCAGGAAGCCCTGCGGATGGCCAACG |
| | hFcm | protein | TGGCAGAGAACAGCAGCAGCGACCAGAGGCAGGCCTGT |
| | | nucleotide | AAGAAGCACGAGCTGTATGTCAGCTTCCGAGACCTGGG |
| | | sequence | CTGGCAGGACTGGATCATCGCGCCTGAAGGCTACGCCGC |
| | | | CTACTACTGTGAGGGGGAGTGTGCCTTCCCTCTGAACTC |
| | | | CTACATGAACGCCACCAACCACGCCATCGTGCAGACGCT |
| | | | GGTCCACTTCATCAACCCGGAAACGGTGCCCAAGCCCTG |
| | | | CTGTGCGCCCACGCAGCTCAATGCCATCTCCGTCCTCTA |
| | | | CTTCGATGACAGCTCCAACGTCATCCTGAAGAAATACAG |
| | | | AAACATGGTGGTCCGGGCCTGTGGCTGCCAC |
| | | | GGATCTGGGAGCGCTGACAAAACTCACACATGCCCACC |
| | | | GTGCCCAGCACCTGAAGCTGCCGGGGGACCGTCAGTCTT |
| | | | CCTCTTCCCCCCAAAACCCAAGGACACCCTCATGATCTC |
| | | | CCGGACCCCTGAGGTCACATGCGTGGTGGTGGACGTGA |
| | | | GCCACGAAGACCCTGAGGTCAAGTTCAACTGGTACGTG |
| | | | GACGGCGTGGAGGTGCATAATGCCAAGACAAAGCCGCG |
| | | | GGAGGAGCAGTACAACAGCACGTACCGTGTGGTCAGCG |
| | | | TCCTCACCGTCCTGCACCAGGACTGGCTGAATGGCAAGG |
| | | | AGTACAAGTGCAAGGTCTCCAACAAAGCCCTCGGCGCC |
| | | | CCCATCGAGAAAACCATCTCCAAAGCCAAAGGGCAGCC |
| | | | CCGAGAACCACAGGTGTACACCCTGCCCCCATCCCGGGA |
| | | | TGAGCTGACCAAGAACCAGGTCAGCCTGACCTGCCTGGT |
| | | | CAAAGGCTTCTATCCCAGCGACATCGCCGTGGAGTGGGA |
| | | | GAGCAATGGGCAGCCGGAGAACAACTACAAGACCACGC |
| | | | CTCCCGTGCTGGACTCCGACGGCTCCTTCTTCCTCTACAG |
| | | | CAAGCTCACCGTGGACAAGAGCAGGTGGCAGCAGGGGA |
| | | | ACGTCTTCTCATGCTCCGTGATGCATGAGGCTCTGCACA |
| | | | ACCACTACACGCAGAAaAGCCTCTCCCTGTCTCCGGGTA |
| | | | AA |
| 54 | FGF-17 | Full length | TQGENHPSPNFNQYVRDQGAMTDQLSRRQIREYQLYSRTS |
| | | of human | GKHVQVTGRRISATAEDGNKFAKLIVETDTFGSRVRIKGAE |
| | | FGF-17 | SEKYICMNKRGKLIGKPSGKSKDCVFTEIVLENNYTAFQNA |
| | | amino acid | RHEGWFMAFTRQGRPRQASRSRQNQREAHFIKRLYQGQLP |
| | | sequence | FPNHAEKQKQFEFVGSAPTRRTKRTRRPQPLT |
| 55 | FGF- | Human | TQGENHPSPNFNQYVRDQGAMTDQLSRRQIREYQLYSRTS |
| | 17d204- | FGF-17 | GKHVQVTGRRISATAEDGNKFAKLIVETDTFGSRVRIKGAE |
| | 216 | AA204-216 | SEKYICMNKRGKLIGKPSGKSKDCVFTEIVLENNYTAFQNA |
| | | deletion | RHEGWFMAFTRQGRPRQASRSRQNQREAHFIKRLYQGQLP |
| | | mutant | FPNHAEKQKQFEFVGSAPT |
| | | amino acid | |
| | | sequence | |
| 56 | FGF- | Human | TQGENHPSPNFNQYVRDQGAMTDQLSRRQIREYQLYSRTS |
| | 17d181- | FGF-17 | GKHVQVTGRRISATAEDGNKFAKLIVETDTFGSRVRIKGAE |
| | 216 | AA181-216 | SEKYICMNKRGKLIGKPSGKSKDCVFTEIVLENNYTAFQNA |
| | | deletion | RHEGWFMAFTRQGRPRQASRSRQNQREAHFIKRLYQ |
| | | mutant | |
| | | amino acid | |
| | | sequence | |
| 57 | FGF- | Human | TQGENHPSPNFNQYVRDQGAMTDQLSRRQIREYQLYSRTS |
| | 17R204Q | FGF-17 | GKHVQVTGRRISATAEDGNKFAKLIVETDTFGSRVRIKGAE |
| | K207Q | R204Q | SEKYICMNKRGKLIGKPSGKSKDCVFTEIVLENNYTAFQNA |
| | | K207Q | RHEGWFMAFTRQGRPRQASRSRQNQREAHFIKRLYQGQLP |
| | | mutant | FPNHAEKQKQFEFVGSAPTQRTQRTRRPQPLT |
| | | amino acid | |
| | | sequence | |
| 58 | FGF- | Human | TQGENHPSPNFNQYVRDQGAMTDQLSRRQIREYQLYSRTS |
| | 17d197- | FGF-17 | GKHVQVTGRRISATAEDGNKFAKLIVETDTFGSRVRIKGAE |
| | 216 | AA197-216 | SEKYICMNKRGKLIGKPSGKSKDCVFTEIVLENNYTAFQNA |
| | | deletion | RHEGWFMAFTRQGRPRQASRSRQNQREAHFIKRLYQGQLP |
| | | mutant | FPNHAEKQKQFE |
| | | amino acid | |
| | | sequence | |
| 59 | FGF- | Human | TQGENHPSPNFNQYVRDQGAMTDQLSRRQIREYQLYSRTS |
| | 17K191A | FGF-17 | GKHVQVTGRRISATAEDGNKFAKLIVETDTFGSRVRIKGAE |
| | K193AS200A | K191A | SEKYICMNKRGKLIGKPSGKSKDCVFTEIVLENNYTAFQNA |
| | | K193A | RHEGWFMAFTRQGRPRQASRSRQNQREAHFIKRLYQGQLP |
| | | S200A | FPNHAEAQAQFEFVGAAPTRRTKRTRRPQPLT |
| | | mutant | |
| | | amino acid | |
| | | sequence | |
| 60 | FGF-17- | Full length | TQGENHPSPNFNQYVRDQGAMTDQLSRRQIREYQLYSRTS |
| | linker1- | of human | GKHVQVTGRRISATAEDGNKFAKLIVETDTFGSRVRIKGAE |
| | hFcm | FGF-17- | SEKYICMNKRGKLIGKPSGKSKDCVFTEIVLENNYTAFQNA |
| | | linker1- | RHEGWFMAFTRQGRPRQASRSRQNQREAHFIKRLYQGQLP |
| | | hFcm amino | FPNHAEKQKQFEFVGSAPTRRTKRTRRPQPLTGSGSDKTHT |
| | | acid | CPPCPAPEAAGGPSVFLFPPKPKDTLMISRTPEVTCVVVDVS |
| | | sequence | HEDPEVKFNWYVDGVEVHNAKTKPREEQYNSTYRVVSVL |
| | | | TVLHQDWLNGKEYKCKVSNKALGAPIEKTISKAKGQPREP |
| | | | QVYTLPPSRDELTKNQVSLTCLVKGFYPSDIAVEWESNGQP |
| | | | ENNYKTTPPVLDSDGSFFLYSKLTVDKSRWQQGNVFSCSV |
| | | | MHEALHNHYTQKSLSLSPGK |
| 61 | FGF- | Human | TQGENHPSPNFNQYVRDQGAMTDQLSRRQIREYQLYSRTS |
| | 17d204- | FGF-17 | GKHVQVTGRRISATAEDGNKFAKLIVETDTFGSRVRIKGAE |
| | 216- | AA204-216 | SEKYICMNKRGKLIGKPSGKSKDCVFTEIVLENNYTAFQNA |
| | linker1- | deletion | RHEGWFMAFTRQGRPRQASRSRQNQREAHFIKRLYQGQLP |
| | hFcm | mutant- | FPNHAEKQKQFEFVGSAPTGSGSDKTHTCPPCPAPEAAGGP |
| | | linker1- | SVFLFPPKPKDTLMISRTPEVTCVVVDVSHEDPEVKFNWYV |
| | | hFcm amino | DGVEVHNAKTKPREEQYNSTYRVVSVLTVLHQDWLNGKE |
| | | acid | YKCKVSNKALGAPIEKTISKAKGQPREPQVYTLPPSRDELT |
| | | sequence | KNQVSLTCLVKGFYPSDIAVEWESNGQPENNYKTTPPVLD |
| | | | SDGSFFLYSKLTVDKSRWQQGNVFSCSVMHEALHNHYTQ |
| | | | KSLSLSPGK |
| 62 | FGF- | Human | TQGENHPSPNFNQYVRDQGAMTDQLSRRQIREYQLYSRTS |
| | 17d181- | FGF-17 | GKHVQVTGRRISATAEDGNKFAKLIVETDTFGSRVRIKGAE |
| | 216- | AA181-216 | SEKYICMNKRGKLIGKPSGKSKDCVFTEIVLENNYTAFQNA |
| | linker1- | deletion | RHEGWFMAFTRQGRPRQASRSRQNQREAHFIKRLYQGSGS |
| | hFcm | mutant- | DKTHTCPPCPAPEAAGGPSVFLFPPKPKDTLMISRTPEVTCV |
| | | linker1- | VVDVSHEDPEVKFNWYVDGVEVHNAKTKPREEQYNSTYR |
| | | hFcm amino | VVSVLTVLHQDWLNGKEYKCKVSNKALGAPIEKTISKAKG |
| | | acid | QPREPQVYTLPPSRDELTKNQVSLTCLVKGFYPSDIAVEWE |
| | | sequence | SNGQPENNYKTTPPVLDSDGSFFLYSKLTVDKSRWQQGNV |
| | | | FSCSVMHEALHNHYTQKSLSLSPGK |
| 63 | FGF- | Human | TQGENHPSPNFNQYVRDQGAMTDQLSRRQIREYQLYSRTS |
| | 17R204Q | FGF-17 | GKHVQVTGRRISATAEDGNKFAKLIVETDTFGSRVRIKGAE |
| | K207Q- | R204Q | SEKYICMNKRGKLIGKPSGKSKDCVFTEIVLENNYTAFQNA |
| | linker1- | K207Q | RHEGWFMAFTRQGRPRQASRSRQNQREAHFIKRLYQGQLP |
| | hFcm | mutant- | FPNHAEKQKQFEFVGSAPTQRTQRTRRPQPLTGSGSDKTHT |
| | | linker1- | CPPCPAPEAAGGPSVFLFPPKPKDTLMISRTPEVTCVVVDVS |
| | | hFcm amino | HEDPEVKFNWYVDGVEVHNAKTKPREEQYNSTYRVVSVL |
| | | acid | TVLHQDWLNGKEYKCKVSNKALGAPIEKTISKAKGQPREP |
| | | sequence | QVYTLPPSRDELTKNQVSLTCLVKGFYPSDIAVEWESNGQP |
| | | | ENNYKTTPPVLDSDGSFFLYSKLTVDKSRWQQGNVFSCSV |
| | | | MHEALHNHYTQKSLSLSPGK |
| 64 | FGF- | Human | TQGENHPSPNFNQYVRDQGAMTDQLSRRQIREYQLYSRTS |
| | 17d197- | FGF17 | GKHVQVTGRRISATAEDGNKFAKLIVETDTFGSRVRIKGAE |
| | 216- | AA197-216 | SEKYICMNKRGKLIGKPSGKSKDCVFTEIVLENNYTAFQNA |
| | linker1- | deletion | RHEGWFMAFTRQGRPRQASRSRQNQREAHFIKRLYQGQLP |
| | hFcm | mutant- | FPNHAEKQKQFEGSGSDKTHTCPPCPAPEAAGGPSVFLFPP |
| | | linker1- | KPKDTLMISRTPEVTCVVVDVSHEDPEVKFNWYVDGVEV |
| | | hFcm | HNAKTKPREEQYNSTYRVVSVLTVLHQDWLNGKEYKCKV |
| | | | SNKALGAPIEKTISKAKGQPREPQVYTLPPSRDELTKNQVS |
| | | | LTCLVKGFYPSDIAVEWESNGQPENNYKTTPPVLDSDGSFF |
| | | | LYSKLTVDKSRWQQGNVFSCSVMHEALHNHYTQKSLSLSP |
| | | | GK |
| 65 | FGF- | Human | TQGENHPSPNFNQYVRDQGAMTDQLSRRQIREYQLYSRTS |
| | 17K191A | FGF-17 | GKHVQVTGRRISATAEDGNKFAKLIVETDTFGSRVRIKGAE |
| | K193AS200A- | K191A | SEKYICMNKRGKLIGKPSGKSKDCVFTEIVLENNYTAFQNA |
| | linker1- | K193A | RHEGWFMAFTRQGRPRQASRSRQNQREAHFIKRLYQGQLP |
| | hFcm | S200A | FPNHAEAQAQFEFVGAAPTRRTKRTRRPQPLTGSGSDKTH |
| | | mutant- | TCPPCPAPEAAGGPSVFLFPPKPKDTLMISRTPEVTCVVVD |
| | | linker1- | VSHEDPEVKFNWYVDGVEVHNAKTKPREEQYNSTYRVVS |
| | | hFcm | VLTVLHQDWLNGKEYKCKVSNKALGAPIEKTISKAKGQPR |
| | | | EPQVYTLPPSRDELTKNQVSLTCLVKGFYPSDIAVEWESNG |
| | | | QPENNYKTTPPVLDSDGSFFLYSKLTVDKSRWQQGNVFSC |
| | | | SVMHEALHNHYTQKSLSLSPGK |
| 66 | FGF-17- | Full length | TQGENHPSPNFNQYVRDQGAMTDQLSRRQIREYQLYSRTS |
| | linker2- | of human | GKHVQVTGRRISATAEDGNKFAKLIVETDTFGSRVRIKGAE |
| | hFcm | FGF17- | SEKYICMNKRGKLIGKPSGKSKDCVFTEIVLENNYTAFQNA |
| | | linker2- | RHEGWFMAFTRQGRPRQASRSRQNQREAHFIKRLYQGQLP |
| | | hFcm | FPNHAEKQKQFEFVGSAPTRRTKRTRRPQPLTGSGSADKTH |
| | | | TCPPCPAPEAAGGPSVFLFPPKPKDTLMISRTPEVTCVVVD |
| | | | VSHEDPEVKFNWYVDGVEVHNAKTKPREEQYNSTYRVVS |
| | | | VLTVLHQDWLNGKEYKCKVSNKALGAPIEKTISKAKGQPR |
| | | | EPQVYTLPPSRDELTKNQVSLTCLVKGFYPSDIAVEWESNG |
| | | | QPENNYKTTPPVLDSDGSFFLYSKLTVDKSRWQQGNVFSC |
| | | | SVMHEALHNHYTQKSLSLSPGK |
| 67 | FGF- | Human | TQGENHPSPNFNQYVRDQGAMTDQLSRRQIREYQLYSRTS |
| | 17d204- | FGF17 | GKHVQVTGRRISATAEDGNKFAKLIVETDTFGSRVRIKGAE |
| | 216- | AA204-216 | SEKYICMNKRGKLIGKPSGKSKDCVFTEIVLENNYTAFQNA |
| | linker2- | deletion | RHEGWFMAFTRQGRPRQASRSRQNQREAHFIKRLYQGQLP |
| | hFcm | mutant- | FPNHAEKQKQFEFVGSAPTGSGSADKTHTCPPCPAPEAAG |
| | | linker2- | GPSVFLFPPKPKDTLMISRTPEVTCVVVDVSHEDPEVKFNW |
| | | hFcm | YVDGVEVHNAKTKPREEQYNSTYRVVSVLTVLHQDWLNG |
| | | | KEYKCKVSNKALGAPIEKTISKAKGQPREPQVYTLPPSRDE |
| | | | LTKNQVSLTCLVKGFYPSDIAVEWESNGQPENNYKTTPPV |
| | | | LDSDGSFFLYSKLTVDKSRWQQGNVFSCSVMHEALHNHY |
| | | | TQKSLSLSPGK |
| 68 | FGF- | Human | TQGENHPSPNFNQYVRDQGAMTDQLSRRQIREYQLYSRTS |
| | 17d181- | FGF17 | GKHVQVTGRRISATAEDGNKFAKLIVETDTFGSRVRIKGAE |
| | 216- | AA181-216 | SEKYICMNKRGKLIGKPSGKSKDCVFTEIVLENNYTAFQNA |
| | linker2- | deletion | RHEGWFMAFTRQGRPRQASRSRQNQREAHFIKRLYQGSGS |
| | hFcm | mutant- | ADKTHTCPPCPAPEAAGGPSVFLFPPKPKDTLMISRTPEVTC |
| | | linker2- | VVVDVSHEDPEVKFNWYVDGVEVHNAKTKPREEQYNSTY |
| | | hFcm | RVVSVLTVLHQDWLNGKEYKCKVSNKALGAPIEKTISKAK |
| | | | GQPREPQVYTLPPSRDELTKNQVSLTCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| 69 | FGF- | Human | TQGENHPSPNFNQYVRDQGAMTDQLSRRQIREYQLYSRTS |
| | 17R204Q | FGF-17 | GKHVQVTGRRISATAEDGNKFAKLIVETDTFGSRVRIKGAE |
| | K207Q- | R204Q | SEKYICMNKRGKLIGKPSGKSKDCVFTEIVLENNYTAFQNA |
| | linker2- | K207Q | RHEGWFMAFTRQGRPRQASRSRQNQREAHFIKRLYQGQLP |
| | hFcm | mutant- | FPNHAEKQKQFEFVGSAPTQRTQRTRRPQPLTGSGSADKT |
| | | linker2- | HTCPPCPAPEAAGGPSVFLFPPKPKDTLMISRTPEVTCVVV |
| | | hFcm | DVSHEDPEVKFNWYVDGVEVHNAKTKPREEQYNSTYRVV |
| | | | SVLTVLHQDWLNGKEYKCKVSNKALGAPIEKTISKAKGQP |
| | | | REPQVYTLPPSRDELTKNQVSLTCLVKGFYPSDIAVEWESN |
| | | | GQPENNYKTTPPVLDSDGSFFLYSKLTVDKSRWQQGNVFS |
| | | | CSVMHEALHNHYTQKSLSLSPGK |
| 70 | FGF- | Human | TQGENHPSPNFNQYVRDQGAMTDQLSRRQIREYQLYSRTS |
| | 17d197- | FGF17 | GKHVQVTGRRISATAEDGNKFAKLIVETDTFGSRVRIKGAE |
| | 216- | AA197-216 | SEKYICMNKRGKLIGKPSGKSKDCVFTEIVLENNYTAFQNA |
| | linker2- | deletion | RHEGWFMAFTRQGRPRQASRSRQNQREAHFIKRLYQGQLP |
| | hFcm | mutant- | FPNHAEKQKQFEGSGSADKTHTCPPCPAPEAAGGPSVFLFP |
| | | linker2- | PKPKDTLMISRTPEVTCVVVDVSHEDPEVKFNWYVDGVEV |
| | | hFcm | HNAKTKPREEQYNSTYRVVSVLTVLHQDWLNGKEYKCKV |
| | | | SNKALGAPIEKTISKAKGQPREPQVYTLPPSRDELTKNQVS |
| | | | LTCLVKGFYPSDIAVEWESNGQPENNYKTTPPVLDSDGSFF |
| | | | LYSKLTVDKSRWQQGNVFSCSVMHEALHNHYTQKSLSLSP |
| | | | GK |
| 71 | FGF- | Human | TQGENHPSPNFNQYVRDQGAMTDQLSRRQIREYQLYSRTS |
| | 17K191A | FGF-17 | GKHVQVTGRRISATAEDGNKFAKLIVETDTFGSRVRIKGAE |
| | K193AS200A- | K191A | SEKYICMNKRGKLIGKPSGKSKDCVFTEIVLENNYTAFQNA |
| | linker2- | K193A | RHEGWFMAFTRQGRPRQASRSRQNQREAHFIKRLYQGQLP |
| | hFcm | S200A | FPNHAEAQAQFEFVGAAPTRRTKRTRRPQPLTGSGSADKT |
| | | mutant- | HTCPPCPAPEAAGGPSVFLFPPKPKDTLMISRTPEVTCVVV |
| | | linker2- | DVSHEDPEVKFNWYVDGVEVHNAKTKPREEQYNSTYRVV |
| | | hFcm | SVLTVLHQDWLNGKEYKCKVSNKALGAPIEKTISKAKGQP |
| | | | REPQVYTLPPSRDELTKNQVSLTCLVKGFYPSDIAVEWESN |
| | | | GQPENNYKTTPPVLDSDGSFFLYSKLTVDKSRWQQGNVFS |
| | | | CSVMHEALHNHYTQKSLSLSPGK |
| 72 | 6xHis- | His tagged | HHHHHHSGDAHKSEVAHRFKDLGEENFKALVLIAFAQYL |
| | HSA- | HSA fusion | QQCPFEDHVKLVNEVTEFAKTCVADESAENCDKSLHTLFG |
| | linker3- | FGF17 with | DKLCTVATLRETYGEMADCCAKQEPERNECFLQHKDDNP |
| | FGF17 | a long | NLPRLVRPEVDVMCTAFHDNEETFLKKYLYEIARRHPYFY |
| | | linker | APELLFFAKRYKAAFTECCQAADKAACLLPKLDELRDEGK |
| | | | ASSAKQRLKCASLQKFGERAFKAWAVARLSQRFPKAEFAE |
| | | | VSKLVTDLTKVHTECCHGDLLECADDRADLAKYICENQDS |
| | | | ISSKLKECCEKPLLEKSHCIAEVENDEMPADLPSLAADFVES |
| | | | KDVCKNYAEAKDVFLGMFLYEYARRHPDYSVVLLLRLAK |
| | | | TYETTLEKCCAAADPHECYAKVFDEFKPLVEEPQNLIKQN |
| | | | CELFEQLGEYKFQNALLVRYTKKVPQVSTPTLVEVSRNLG |
| | | | KVGSKCCKHPEAKRMPCAEDYLSVVLNQLCVLHEKTPVS |
| | | | DRVTKCCTESLVNRRPCFSALEVDETYVPKEFNAETFTFHA |
| | | | DICTLSEKERQIKKQTALVELVKHKPKATKEQLKAVMDDF |
| | | | AAFVEKCCKADDKETCFAEEGKKLVAASQAALGLGGGGS |
| | | | GGGGSGGGGSTQGENHPSPNFNQYVRDQGAMTDQLSRRQ |
| | | | IREYQLYSRTSGKHVQVTGRRISATAEDGNKFAKLIVETDT |
| | | | FGSRVRIKGAESEKYICMNKRGKLIGKPSGKSKDCVFTEIV |
| | | | LENNYTAFQNARHEGWFMAFTRQGRPRQASRSRQNQREA |
| | | | HFIKRLYQGQLPFPNHAEKQKQFEFVGSAPTRRTKRTRRPQ |
| | | | PLT |
| 73 | FGF17- | Human | TQGENHPSPNFNQYVRDQGAMTDQLSRRQIREYQLYSRTS |
| | linker3- | FGF17- | GKHVQVTGRRISATAEDGNKFAKLIVETDTFGSRVRIKGAE |
| | hFc4 | linker3- | SEKYICMNKRGKLIGKPSGKSKDCVFTEIVLENNYTAFQNA |
| | | hFc4 | RHEGWFMAFTRQGRPRQASRSRQNQREAHFIKRLYQGQLP |
| | | | FPNHAEKQKQFEFVGSAPTRRTKRTRRPQPLTGGGGSGGG |
| | | | GSGGGGSESKYGPPCPPCPAPEFLGGPSVFLFPPKPKDTLMI |
| | | | SRTPEVTCVVVDVSQEDPEVQFNWYVDGVEVHNAKTKPR |
| | | | EEQFNSTYRVVSVLTVLHQDWLNGKEYKCKVSNKGLPSSI |
| | | | EKTISKAKGQPREPQVYTLPPSQEEMTKNQVSLTCLVKGFY |
| | | | PSDIAVEWESNGQPENNYKTTPPVLDSDGSFFLYSRLTVDK |
| | | | SRWQEGNVFSCSVMHEALHNHYTQKSLSLSLGK |
| 74 | FGF17- | Human | TQGENHPSPNFNQYVRDQGAMTDQLSRRQIREYQLYSRTS |
| | hFc4 | FGF17- | GKHVQVTGRRISATAEDGNKFAKLIVETDTFGSRVRIKGAE |
| | | hFc4 | SEKYICMNKRGKLIGKPSGKSKDCVFTEIVLENNYTAFQNA |
| | | | RHEGWFMAFTRQGRPRQASRSRQNQREAHFIKRLYQGQLP |
| | | | FPNHAEKQKQFEFVGSAPTRRTKRTRRPQPLTESKYGPPCP |
| | | | PCPAPEFLGGPSVFLFPPKPKDTLMISRTPEVTCVVVDVSQE |
| | | | DPEVQFNWYVDGVEVHNAKTKPREEQFNSTYRVVSVLTV |
| | | | LHQDWLNGKEYKCKVSNKGLPSSIEKTISKAKGQPREPQV |
| | | | YTLPPSQEEMTKNQVSLTCLVKGFYPSDIAVEWESNGQPE |
| | | | NNYKTTPPVLDSDGSFFLYSRLTVDKSRWQEGNVFSCSVM |
| | | | HEALHNHYTQKSLSLSLGK |
| 75 | hFc4L- | hFc4- | ESKYGPPCPPCPAPEFLGGPSVFLFPPKPKDTLMISRTPEVTC |
| | FGF17 | linker3- | VVVDVSQEDPEVQFNWYVDGVEVHNAKTKPREEQFNSTY |
| | | human | RVVSVLTVLHQDWLNGKEYKCKVSNKGLPSSIEKTISKAK |
| | | FGF17 | GQPREPQVYTLPPSQEEMTKNQVSLTCLVKGFYPSDIAVE |
| | | | WESNGQPENNYKTTPPVLDSDGSFFLYSRLTVDKSRWQEG |
| | | | NVFSCSVMHEALHNHYTQKSLSLSLGKGGGGSGGGGSGG |
| | | | GGSTQGENHPSPNFNQYVRDQGAMTDQLSRRQIREYQLYS |
| | | | RTSGKHVQVTGRRISATAEDGNKFAKLIVETDTFGSRVRIK |
| | | | GAESEKYICMNKRGKLIGKPSGKSKDCVFTEIVLENNYTAF |
| | | | QNARHEGWFMAFTRQGRPRQASRSRQNQREAHFIKRLYQ |
| | | | GQLPFPNHAEKQKQFEFVGSAPTRRTKRTRRPQPLT |
| 76 | IGF2 | human | AYRPSETLCGGELVDTLQFVCGDRGFYFSRPASRVSRRSRG |
| | | IGF2 amino | IVEECCFRSCDLALLETYCATPAKSE |
| | | acid | |
| | | sequence | |
| 77 | IGF2- | | AYRPSETLCGGELVDTLQFVCGDRGFYFSRPASRVSRRSRG |
| | linker1- | | IVEECCFRSCDLALLETYCATPAKSEGSGSDKTHTCPPCPAP |
| | hFcm | | EAAGGPSVFLFPPKPKDTLMISRTPEVTCVVVDVSHEDPEV |
| | | | KFNWYVDGVEVHNAKTKPREEQYNSTYRVVSVLTVLHQD |
| | | | WLNGKEYKCKVSNKALGAPIEKTISKAKGQPREPQVYTLP |
| | | | PSRDELTKNQVSLTCLVKGFYPSDIAVEWESNGQPENNYK |
| | | | TTPPVLDSDGSFFLYSKLTVDKSRWQQGNVFSCSVMHEAL |
| | | | HNHYTQKSLSLSPGK |
| 78 | IGF2- | | AYRPSETLCGGELVDTLQFVCGDRGFYFSRPASRVSRRSRG |
| | linker2- | | IVEECCFRSCDLALLETYCATPAKSEGSGSADKTHTCPPCP |
| | hFcm | | APEAAGGPSVFLFPPKPKDTLMISRTPEVTCVVVDVSHEDP |
| | | | EVKFNWYVDGVEVHNAKTKPREEQYNSTYRVVSVLTVLH |
| | | | QDWLNGKEYKCKVSNKALGAPIEKTISKAKGQPREPQVYT |
| | | | LPPSRDELTKNQVSLTCLVKGFYPSDIAVEWESNGQPENNY |
| | | | KTTPPVLDSDGSFFLYSKLTVDKSRWQQGNVFSCSVMHEA |
| | | | LHNHYTQKSLSLSPGK |
| 79 | IGF2 Big | Full-length | MGIPMGKSMLVLLTFLAFASCCIAAYRPSETLCGGELVDTL |
| | | human | QFVCGDRGFYFSRPASRVSRRSRGIVEECCFRSCDLALLET |
| | | IGF2 | YCATPAKSERDVSTPPTVLPDNFPRYPVGKFFQYDTWKQS |
| | | | TQRLRRGLPALLRARRGHVLAKELEAFREAKRHRPLIALPT |
| | | | QDPAHGGAPPEMASNRK |
| 80 | 6xHis- | His tagged | HHHHHHSGDAHKSEVAHRFKDLGEENFKALVLIAFAQYL |
| | HSA- | HSA fusion | QQCPFEDHVKLVNEVTEFAKTCVADESAENCDKSLHTLFG |
| | linker3- | IGF2 with a | DKLCTVATLRETYGEMADCCAKQEPERNECFLQHKDDNP |
| | IGF2 | long linker | NLPRLVRPEVDVMCTAFHDNEETFLKKYLYEIARRHPYFY |
| | | | APELLFFAKRYKAAFTECCQAADKAACLLPKLDELRDEGK |
| | | | ASSAKQRLKCASLQKFGERAFKAWAVARLSQRFPKAEFAE |
| | | | VSKLVTDLTKVHTECCHGDLLECADDRADLAKYICENQDS |
| | | | ISSKLKECCEKPLLEKSHCIAEVENDEMPADLPSLAADFVES |
| | | | KDVCKNYAEAKDVFLGMFLYEYARRHPDYSVVLLLRLAK |
| | | | TYETTLEKCCAAADPHECYAKVFDEFKPLVEEPQNLIKQN |
| | | | CELFEQLGEYKFQNALLVRYTKKVPQVSTPTLVEVSRNLG |
| | | | KVGSKCCKHPEAKRMPCAEDYLSVVLNQLCVLHEKTPVS |
| | | | DRVTKCCTESLVNRRPCFSALEVDETYVPKEFNAETFTFHA |
| | | | DICTLSEKERQIKKQTALVELVKHKPKATKEQLKAVMDDF |
| | | | AAFVEKCCKADDKETCFAEEGKKLVAASQAALGLGGGGS |
| | | | GGGGSGGGGSAYRPSETLCGGELVDTLQFVCGDRGFYFSR |
| | | | PASRVSRRSRGIVEECCFRSCDLALLETYCATPAKSE |
| 81 | 6xHis- | His tagged | HHHHHHSGDAHKSEVAHRFKDLGEENFKALVLIAFAQYL |
| | HSA- | HSA fusion | QQCPFEDHVKLVNEVTEFAKTCVADESAENCDKSLHTLFG |
| | linker3- | IGF2 R61A | DKLCTVATLRETYGEMADCCAKQEPERNECFLQHKDDNP |
| | IGF2R61A | mutant with | NLPRLVRPEVDVMCTAFHDNEETFLKKYLYEIARRHPYFY |
| | | a long | APELLFFAKRYKAAFTECCQAADKAACLLPKLDELRDEGK |
| | | linker | ASSAKQRLKCASLQKFGERAFKAWAVARLSQRFPKAEFAE |
| | | | VSKLVTDLTKVHTECCHGDLLECADDRADLAKYICENQDS |
| | | | ISSKLKECCEKPLLEKSHCIAEVENDEMPADLPSLAADFVES |
| | | | KDVCKNYAEAKDVFLGMFLYEYARRHPDYSVVLLLRLAK |
| | | | TYETTLEKCCAAADPHECYAKVFDEFKPLVEEPQNLIKQN |
| | | | CELFEQLGEYKFQNALLVRYTKKVPQVSTPTLVEVSRNLG |
| | | | KVGSKCCKHPEAKRMPCAEDYLSVVLNQLCVLHEKTPVS |
| | | | DRVTKCCTESLVNRRPCFSALEVDETYVPKEFNAETFTFHA |
| | | | DICTLSEKERQIKKQTALVELVKHKPKATKEQLKAVMDDF |
| | | | AAFVEKCCKADDKETCFAEEGKKLVAASQAALGLGGGGS |
| | | | GGGGSGGGGSAYRPSETLCGGELVDTLQFVCGDRGFYFSR |
| | | | PASRVSARSRGIVEECCFRSCDLALLETYCATPAKSE |
| 82 | 6xHis- | His tagged | HHHHHHSGDAHKSEVAHRFKDLGEENFKALVLIAFAQYL |
| | HSA- | HSA fusion | QQCPFEDHVKLVNEVTEFAKTCVADESAENCDKSLHTLFG |
| | linker3- | IGF2 R61Q | DKLCTVATLRETYGEMADCCAKQEPERNECFLQHKDDNP |
| | IGF2R61Q | mutant with | NLPRLVRPEVDVMCTAFHDNEETFLKKYLYEIARRHPYFY |
| | | a long | APELLFFAKRYKAAFTECCQAADKAACLLPKLDELRDEGK |
| | | linker | ASSAKQRLKCASLQKFGERAFKAWAVARLSQRFPKAEFAE |
| | | | VSKLVTDLTKVHTECCHGDLLECADDRADLAKYICENQDS |
| | | | ISSKLKECCEKPLLEKSHCIAEVENDEMPADLPSLAADFVES |
| | | | KDVCKNYAEAKDVFLGMFLYEYARRHPDYSVVLLLRLAK |
| | | | TYETTLEKCCAAADPHECYAKVFDEFKPLVEEPQNLIKQN |
| | | | CELFEQLGEYKFQNALLVRYTKKVPQVSTPTLVEVSRNLG |
| | | | KVGSKCCKHPEAKRMPCAEDYLSVVLNQLCVLHEKTPVS |
| | | | DRVTKCCTESLVNRRPCFSALEVDETYVPKEFNAETFTFHA |
| | | | DICTLSEKERQIKKQTALVELVKHKPKATKEQLKAVMDDF |
| | | | AAFVEKCCKADDKETCFAEEGKKLVAASQAALGLGGGGS |
| | | | GGGGSGGGGSAYRPSETLCGGELVDTLQFVCGDRGFYFSR |
| | | | PASRVSQRSRGIVEECCFRSCDLALLETYCATPAKSE |
| 83 | 6xHis- | His tagged | HHHHHHSGDAHKSEVAHRFKDLGEENFKALVLIAFAQYL |
| | HSA- | HSA fusion | QQCPFEDHVKLVNEVTEFAKTCVADESAENCDKSLHTLFG |
| | linker3- | IGF2 R64A | DKLCTVATLRETYGEMADCCAKQEPERNECFLQHKDDNP |
| | IGF2R64A | mutant with | NLPRLVRPEVDVMCTAFHDNEETFLKKYLYEIARRHPYFY |
| | | a long | APELLFFAKRYKAAFTECCQAADKAACLLPKLDELRDEGK |
| | | linker | ASSAKQRLKCASLQKFGERAFKAWAVARLSQRFPKAEFAE |
| | | | VSKLVTDLTKVHTECCHGDLLECADDRADLAKYICENQDS |
| | | | ISSKLKECCEKPLLEKSHCIAEVENDEMPADLPSLAADFVES |
| | | | KDVCKNYAEAKDVFLGMFLYEYARRHPDYSVVLLLRLAK |
| | | | TYETTLEKCCAAADPHECYAKVFDEFKPLVEEPQNLIKQN |
| | | | CELFEQLGEYKFQNALLVRYTKKVPQVSTPTLVEVSRNLG |
| | | | KVGSKCCKHPEAKRMPCAEDYLSVVLNQLCVLHEKTPVS |
| | | | DRVTKCCTESLVNRRPCFSALEVDETYVPKEFNAETFTFHA |
| | | | DICTLSEKERQIKKQTALVELVKHKPKATKEQLKAVMDDF |
| | | | AAFVEKCCKADDKETCFAEEGKKLVAASQAALGLGGGGS |
| | | | GGGGSGGGGSAYRPSETLCGGELVDTLQFVCGDRGFYFSR |
| | | | PASRVSRRSAGIVEECCFRSCDLALLETYCATPAKSE |
| 84 | 6xHis- | His tagged | HHHHHHSGDAHKSEVAHRFKDLGEENFKALVLIAFAQYL |
| | HSA- | HSA fusion | QQCPFEDHVKLVNEVTEFAKTCVADESAENCDKSLHTLFG |
| | linker3- | IGF2 R64Q | DKLCTVATLRETYGEMADCCAKQEPERNECFLQHKDDNP |
| | IGF2R64Q | mutant with | NLPRLVRPEVDVMCTAFHDNEETFLKKYLYEIARRHPYFY |
| | | a long | APELLFFAKRYKAAFTECCQAADKAACLLPKLDELRDEGK |
| | | linker | ASSAKQRLKCASLQKFGERAFKAWAVARLSQRFPKAEFAE |
| | | | VSKLVTDLTKVHTECCHGDLLECADDRADLAKYICENQDS |
| | | | ISSKLKECCEKPLLEKSHCIAEVENDEMPADLPSLAADFVES |
| | | | KDVCKNYAEAKDVFLGMFLYEYARRHPDYSVVLLLRLAK |
| | | | TYETTLEKCCAAADPHECYAKVFDEFKPLVEEPQNLIKQN |
| | | | CELFEQLGEYKFQNALLVRYTKKVPQVSTPTLVEVSRNLG |
| | | | KVGSKCCKHPEAKRMPCAEDYLSVVLNQLCVLHEKTPVS |
| | | | DRVTKCCTESLVNRRPCFSALEVDETYVPKEFNAETFTFHA |
| | | | DICTLSEKERQIKKQTALVELVKHKPKATKEQLKAVMDDF |
| | | | AAFVEKCCKADDKETCFAEEGKKLVAASQAALGLGGGGS |
| | | | GGGGSGGGGSAYRPSETLCGGELVDTLQFVCGDRGFYFSR |
| | | | PASRVSRRSQGIVEECCFRSCDLALLETYCATPAKSE |
| 85 | IGF2- | Human | AYRPSETLCGGELVDTLQFVCGDRGFYFSRPASRVSRRSRG |
| | linker3- | IGF2- | IVEECCFRSCDLALLETYCATPAKSEGGGGSGGGGSGGGGS |
| | hFc4 | linker3- | ESKYGPPCPPCPAPEFLGGPSVFLFPPKPKDTLMISRTPEVTC |
| | | hFc4 | VVVDVSQEDPEVQFNWYVDGVEVHNAKTKPREEQFNSTY |
| | | | RVVSVLTVLHQDWLNGKEYKCKVSNKGLPSSIEKTISKAK |
| | | | GQPREPQVYTLPPSQEEMTKNQVSLTCLVKGFYPSDIAVE |
| | | | WESNGQPENNYKTTPPVLDSDGSFFLYSRLTVDKSRWQEG |
| | | | NVFSCSVMHEALHNHYTQKSLSLSLGK |
| 86 | IGF2- | Human | AYRPSETLCGGELVDTLQFVCGDRGFYFSRPASRVSRRSRG |
| | hFc4 | IGF2-hFc4 | IVEECCFRSCDLALLETYCATPAKSEESKYGPPCPPCPAPEF |
| | | | LGGPSVFLFPPKPKDTLMISRTPEVTCVVVDVSQEDPEVQF |
| | | | NWYVDGVEVHNAKTKPREEQFNSTYRVVSVLTVLHQDW |
| | | | LNGKEYKCKVSNKGLPSSIEKTISKAKGQPREPQVYTLPPS |
| | | | QEEMTKNQVSLTCLVKGFYPSDIAVEWESNGQPENNYKTT |
| | | | PPVLDSDGSFFLYSRLTVDKSRWQEGNVFSCSVMHEALHN |
| | | | HYTQKSLSLSLGK |
| 87 | hFc4- | hFc4- | ESKYGPPCPPCPAPEFLGGPSVFLFPPKPKDTLMISRTPEVTC |
| | linker3- | linker3- | VVVDVSQEDPEVQFNWYVDGVEVHNAKTKPREEQFNSTY |
| | IGF2 | human | RVVSVLTVLHQDWLNGKEYKCKVSNKGLPSSIEKTISKAK |
| | | IGF2 | GQPREPQVYTLPPSQEEMTKNQVSLTCLVKGFYPSDIAVE |
| | | | WESNGQPENNYKTTPPVLDSDGSFFLYSRLTVDKSRWQEG |
| | | | NVFSCSVMHEALHNHYTQKSLSLSLGKGGGGSGGGGSGG |
| | | | GGSAYRPSETLCGGELVDTLQFVCGDRGFYFSRPASRVSRR |
| | | | SRGIVEECCFRSCDLALLETYCATPAKSE |
| 88 | IGF2R61 | Human | AYRPSETLCGGELVDTLQFVCGDRGFYFSRPASRVSARSRG |
| | A-linker3- | IGF2 R61A | IVEECCFRSCDLALLETYCATPAKSEGGGGSGGGGSGGGGS |
| | hFc4 | point | ESKYGPPCPPCPAPEFLGGPSVFLFPPKPKDTLMISRTPEVTC |
| | | mutant- | VVVDVSQEDPEVQFNWYVDGVEVHNAKTKPREEQFNSTY |
| | | linker3- | RVVSVLTVLHQDWLNGKEYKCKVSNKGLPSSIEKTISKAK |
| | | hFc4 | GQPREPQVYTLPPSQEEMTKNQVSLTCLVKGFYPSDIAVE |
| | | | WESNGQPENNYKTTPPVLDSDGSFFLYSRLTVDKSRWQEG |
| | | | NVFSCSVMHEALHNHYTQKSLSLSLGK |
| 89 | BMP-7 | Mature | STGSKQRSQNRSKTPKNQEALRMANVAENSSSDQRQACK |
| | | form of | KHELYVSFRDLGWQDWIIAPEGYAAYYCEGECAFPLNSYM |
| | | human | NATNHAIVQTLVHFINPETVPKPCCAPTQLNAISVLYFDDSS |
| | | BMP-7 | NVILKKYRNMVVRACGCH |
| | | (AA293- | |
| | | 431) | |
| 90 | BMP-7- | BMP7 | STGSKQRSQNRSKTPKNQEALRMANVAENSSSDQRQACK |
| | linker1- | fusion | KHELYVSFRDLGWQDWIIAPEGYAAYYCEGECAFPLNSYM |
| | hFcm | protein | NATNHAIVQTLVHFINPETVPKPCCAPTQLNAISVLYFDDSS |
| | | | NVILKKYRNMVVRACGCHGSGSDKTHTCPPCPAPEAAGGP |
| | | | SVFLFPPKPKDTLMISRTPEVTCVVVDVSHEDPEVKFNWYV |
| | | | DGVEVHNAKTKPREEQYNSTYRVVSVLTVLHQDWLNGKE |
| | | | YKCKVSNKALGAPIEKTISKAKGQPREPQVYTLPPSRDELT |
| | | | KNQVSLTCLVKGFYPSDIAVEWESNGQPENNYKTTPPVLD |
| | | | SDGSFFLYSKLTVDKSRWQQGNVFSCSVMHEALHNHYTQ |
| | | | KSLSLSPGK |
| 91 | BMP-7- | BMP7 | STGSKQRSQNRSKTPKNQEALRMANVAENSSSDQRQACK |
| | linker2- | fusion | KHELYVSFRDLGWQDWIIAPEGYAAYYCEGECAFPLNSYM |
| | hFcm | protein | NATNHAIVQTLVHFINPETVPKPCCAPTQLNAISVLYFDDSS |
| | | | NVILKKYRNMVVRACGCHGSGSADKTHTCPPCPAPEAAG |
| | | | GPSVFLFPPKPKDTLMISRTPEVTCVVVDVSHEDPEVKFNW |
| | | | YVDGVEVHNAKTKPREEQYNSTYRVVSVLTVLHQDWLNG |
| | | | KEYKCKVSNKALGAPIEKTISKAKGQPREPQVYTLPPSRDE |
| | | | LTKNQVSLTCLVKGFYPSDIAVEWESNGQPENNYKTTPPV |
| | | | LDSDGSFFLYSKLTVDKSRWQQGNVFSCSVMHEALHNHY |
| | | | TQKSLSLSPGK |
| 92 | BMP7 | BMP7 | ACGGTGCCCAAGCCCTGCTGTGCGCCCACGCAGCTCAAT |
| | | knuckle | GCCATCTCCGTCCTCTACTTCGATGACAGCTCCAACGTC |
| | | | ATCCTGAAGAAATACAGA |
| 93 | BMP7 | BMP7 | TVPKPCCAPTQLNAISVLYFDDSSNVILKKYR |
| | | knuckle[A1] | |
| | | [A2] | |

TABLE 3
Linker and Heterologous Sequences

| Seq ID | Sequence | | |
|---|---|---|---|
| No | Name | Description | Sequence |
| 94 | Linker 1 | A short flexible | GGATCGGGATCG |
| | | linker nucleotide | |
| | | sequence | |
| 95 | Linker 2 | A short flexible | GGATCTGGGAGCGCT |
| | | linker nucleotide | |
| | | sequence | |
| 96 | Linker 3 | A long flexible | GGCGGAGGCGGTAGCGGAGGCGGTGGCTCCGGTGGC |
| | | linker nucleotide | GGAGGGTCT |
| | | sequence | |
| 97 | hFcm | Human IgG1 Fc | GACAAAACTCACACATGCCCACCGTGCCCAGCACCT |
| | IgG1 | mutant | GAAGCTGCCGGGGGACCGTCAGTCTTCCTCTTCCCC |
| | | (L234AL235AP329G) | CCAAAACCCAAGGACACCCTCATGATCTCCCGGACC |
| | | nucleotide | CCTGAGGTCACATGCGTGGTGGTGGACGTGAGCCAC |
| | | sequence | GAAGACCCTGAGGTCAAGTTCAACTGGTACGTGGAC |
| | | | GGCGTGGAGGTGCATAATGCCAAGACAAAGCCGCGG |
| | | | GAGGAGCAGTACAACAGCACGTACCGTGTGGTCAGC |
| | | | GTCCTCACCGTCCTGCACCAGGACTGGCTGAATGGC |
| | | | AAGGAGTACAAGTGCAAGGTCTCCAACAAAGCCCTC |
| | | | GGCGCCCCCATCGAGAAAACCATCTCCAAAGCCAAA |
| | | | GGGCAGCCCCGAGAACCACAGGTGTACACCCTGCCC |
| | | | CCATCCCGGGATGAGCTGACCAAGAACCAGGTCAGC |
| | | | CTGACCTGCCTGGTCAAAGGCTTCTATCCCAGCGAC |
| | | | ATCGCCGTGGAGTGGGAGAGCAATGGGCAGCCGGAG |
| | | | AACAACTACAAGACCACGCCTCCCGTGCTGGACTCC |
| | | | GACGGCTCCTTCTTCCTCTACAGCAAGCTCACCGTG |
| | | | GACAAGAGCAGGTGGCAGCAGGGGAACGTCTTCTCA |
| | | | TGCTCCGTGATGCATGAGGCTCTGCACAACCACTAC |
| | | | ACGCAGAAaAGCCTCTCCCTGTCTCCGGGTAAA |
| 98 | hFc4 | Human IgG4 Fc | GAGTCCAAATATGGTCCCCCATGCCCACCCTGCCCA |
| | | with S228P | GCACCTGAGTTCCTGGGGGGACCATCAGTCTTCCTG |
| | | point mutation | TTCCCCCCAAAACCCAAGGACACTCTCATGATCTCC |
| | | nucleotide | CGGACCCCTGAGGTCACGTGCGTGGTGGTGGACGTG |
| | | sequence | AGCCAGGAAGACCCCGAGGTCCAGTTCAACTGGTAC |
| | | | GTGGATGGCGTGGAGGTGCATAATGCCAAGACAAAG |
| | | | CCGCGGGAGGAGCAGTTCAACAGCACGTACCGTGTG |
| | | | GTCAGCGTCCTCACCGTCCTGCACCAGGACTGGCTG |
| | | | AACGGCAAGGAGTACAAGTGCAAGGTCTCCAACAAA |
| | | | GGCCTCCCGTCCTCCATCGAGAAAACCATCTCCAAA |
| | | | GCCAAAGGGCAGCCCCGAGAGCCACAGGTGTACACC |
| | | | CTGCCCCCATCCCAGGAGGAGATGACCAAGAACCAG |
| | | | GTCAGCCTGACCTGCCTGGTCAAAGGCTTCTACCCC |
| | | | AGCGACATCGCCGTGGAGTGGGAGAGCAATGGGCAG |
| | | | CCGGAGAACAACTACAAGACCACGCCTCCCGTGCTG |
| | | | GACTCCGACGGCTCCTTCTTCCTCTACAGCAGGCTC |
| | | | ACCGTGGACAAGAGCAGGTGGCAGGAGGGGAATGTC |
| | | | TTCTCATGCTCCGTGATGCATGAGGCTCTGCACAAC |
| | | | CACTACACACAGAAGAGCCTCTCCCTGTCTCTGGGT |
| | | | AAA |
| 99 | 6xHis | Six Histidine | CACCATCACCATCACCAT |
| | | short peptide | |
| | | nucleotide | |
| | | sequence | |
| 100 | StrepII | Strep-Tactin | TGGAGCCACCCGCAGTTCGAAAAA |
| | | binding peptide | |
| | | nucleotide | |
| | | sequence | |
| 101 | HSA | Full length of | GATGCACACAAGAGTGAGGTTGCTCATCGGTTTAAA |
| | | Human Serum | GATTTGGGAGAAGAAAATTTCAAAGCCTTGGTGTTG |
| | | Albumin | ATTGCCTTTGCTCAGTATCTTCAGCAGTGTCCATTT |
| | | nucleotide | GAAGATCATGTAAAATTAGTGAATGAAGTAACTGAA |
| | | sequence | TTTGCAAAAACATGTGTTGCTGATGAGTCAGCTGAA |
| | | | AATTGTGACAAATCACTTCATACCCTTTTTGGAGAC |
| | | | AAATTATGCACAGTTGCAACTCTTCGTGAAACCTAT |
| | | | GGTGAAATGGCTGACTGCTGTGCAAAACAAGAACCT |
| | | | GAGAGAAATGAATGCTTCTTGCAACACAAAGATGAC |
| | | | AACCCAAACCTCCCCCGATTGGTGAGACCAGAGGTT |
| | | | GATGTGATGTGCACTGCTTTTCATGACAATGAAGAG |
| | | | ACATTTTTGAAAAAATACTTATATGAAATTGCCAGA |
| | | | AGACATCCTTACTTTTATGCCCCGGAACTCCTTTTC |
| | | | TTTGCTAAAAGGTATAAAGCTGCTTTTACAGAATGT |
| | | | TGCCAAGCTGCTGATAAAGCTGCCTGCCTGTTGCCA |
| | | | AAGCTCGATGAACTTCGGGATGAAGGGAAGGCTTCG |
| | | | TCTGCCAAACAGAGACTCAAGTGTGCCAGTCTCCAA |
| | | | AAATTTGGAGAAAGAGCTTTCAAAGCATGGGCAGTA |
| | | | GCTCGCCTGAGCCAGAGATTTCCCAAAGCTGAGTTT |
| | | | GCAGAAGTTTCCAAGTTAGTGACAGATCTTACCAAA |
| | | | GTCCACACGGAATGCTGCCATGGAGATCTGCTTGAA |
| | | | TGTGCTGATGACAGGGCGGACCTTGCCAAGTATATC |
| | | | TGTGAAAATCAAGATTCGATCTCCAGTAAACTGAAG |
| | | | GAATGCTGTGAAAAACCTCTGTTGGAAAAATCCCAC |
| | | | TGCATTGCCGAAGTGGAAAATGATGAGATGCCTGCT |
| | | | GACTTGCCTTCATTAGCTGCTGATTTTGTTGAAAGT |
| | | | AAGGATGTTTGCAAAAACTATGCTGAGGCAAAGGAT |
| | | | GTCTTCCTGGGCATGTTTTTGTATGAATATGCAAGA |
| | | | AGGCATCCTGATTACTCTGTCGTGCTGCTGCTGAGA |
| | | | CTTGCCAAGACATATGAAACCACTCTAGAGAAGTGC |
| | | | TGTGCCGCTGCAGATCCTCATGAATGCTATGCCAAA |
| | | | GTGTTCGATGAATTTAAACCTCTTGTGGAAGAGCCT |
| | | | CAGAATTTAATCAAACAAAATTGTGAGCTTTTTGAG |
| | | | CAGCTTGGAGAGTACAAATTCCAGAATGCGCTATTA |
| | | | GTTCGTTACACCAAGAAAGTACCCCAAGTGTCAACT |
| | | | CCAACTCTTGTAGAGGTCTCAAGAAACCTAGGAAAA |
| | | | GTGGGCAGCAAATGTTGTAAACATCCTGAAGCAAAA |
| | | | AGAATGCCCTGTGCAGAAGACTATCTATCCGTGGTC |
| | | | CTGAACCAGTTATGTGTGTTGCATGAGAAAACGCCA |
| | | | GTAAGTGACAGAGTCACCAAATGCTGCACAGAATCC |
| | | | TTGGTGAACAGGCGACCATGCTTTTCAGCTCTGGAA |
| | | | GTCGATGAAACATACGTTCCCAAAGAGTTTAATGCT |
| | | | GAAACATTCACCTTCCATGCAGATATATGCACACTT |
| | | | TCTGAGAAGGAGAGACAAATCAAGAAACAAACTGCA |
| | | | CTTGTTGAGCTCGTGAAACACAAGCCCAAGGCAACA |
| | | | AAAGAGCAACTGAAAGCTGTTATGGATGATTTCGCA |
| | | | GCTTTTGTAGAGAAGTGCTGCAAGGCTGACGATAAG |
| | | | GAGACCTGCTTTGCCGAGGAGGGTAAAAAACTTGTT |
| | | | GCTGCAAGTCAAGCTGCCTTAGGCTTA |
| 102 | Linker 1 | A short flexible | GSGS |
| | | linker amino | |
| | | acid sequence | |
| 103 | Linker 2 | A short flexible | GSGSA |
| | | linker amino | |
| | | acid sequence | |
| 104 | Linker 3 | A long flexible | GGGGSGGGGSGGGGS |
| | | linker amino | |
| | | acid sequence | |
| 105 | hFcm | Human IgG1 Fc | DKTHTCPPCPAPEAAGGPSVFLFPPKPKDTLMISRT |
| | IgG1 | mutant (L234A | PEVTCVVVDVSHEDPEVKFNWYVDGVEVHNAKTKPR |
| | | L235A P329G) | EEQYNSTYRVVSVLTVLHQDWLNGKEYKCKVSNKAL |
| | | amino acid | GAPIEKTISKAKGQPREPQVYTLPPSRDELTKNQVS |
| | | sequence | LTCLVKGFYPSDIAVEWESNGQPENNYKTTPPVLDS |
| | | | DGSFFLYSKLTVDKSRWQQGNVFSCSVMHEALHNHY |
| | | | TQKSLSLSPGK |
| 106 | hFc4 | Human IgG4 Fc | ESKYGPPCPPCPAPEFLGGPSVFLFPPKPKDTLMIS |
| | | with S228P | RTPEVTCVVVDVSQEDPEVQFNWYVDGVEVHNAKTK |
| | | point mutation | PREEQFNSTYRVVSVLTVLHQDWLNGKEYKCKVSNK |
| | | amino acid | GLPSSIEKTISKAKGQPREPQVYTLPPSQEEMTKNQ |
| | | sequence | VSLTCLVKGFYPSDIAVEWESNGQPENNYKTTPPVL |
| | | | DSDGSFFLYSRLTVDKSRWQEGNVFSCSVMHEALHN |
| | | | HYTQKSLSLSLGK |
| 107 | 6xHis | Six Histidine | HHHHHH |
| | | short peptide | |
| | | amino acid | |
| | | sequence | |
| 108 | StrepII | Strep-Tactin | WSHPQFEK |
| | | binding peptide | |
| | | amino acid | |
| | | sequence | |
| 109 | HSA | Full length of | DAHKSEVAHRFKDLGEENFKALVLIAFAQYLQQCPF |
| | | Human Serum | EDHVKLVNEVTEFAKTCVADESAENCDKSLHTLFGD |
| | | Albumin amino | KLCTVATLRETYGEMADCCAKQEPERNECFLQHKDD |
| | | acid sequence | NPNLPRLVRPEVDVMCTAFHDNEETFLKKYLYEIAR |
| | | | RHPYFYAPELLFFAKRYKAAFTECCQAADKAACLLP |
| | | | KLDELRDEGKASSAKQRLKCASLQKFGERAFKAWAV |
| | | | ARLSQRFPKAEFAEVSKLVTDLTKVHTECCHGDLLE |
| | | | CADDRADLAKYICENQDSISSKLKECCEKPLLEKSH |
| | | | CIAEVENDEMPADLPSLAADFVESKDVCKNYAEAKD |
| | | | VFLGMFLYEYARRHPDYSVVLLLRLAKTYETTLEKC |
| | | | CAAADPHECYAKVFDEFKPLVEEPQNLIKQNCELFE |
| | | | QLGEYKFQNALLVRYTKKVPQVSTPTLVEVSRNLGK |
| | | | VGSKCCKHPEAKRMPCAEDYLSVVLNQLCVLHEKTP |
| | | | VSDRVTKCCTESLVNRRPCFSALEVDETYVPKEFNA |
| | | | ETFTFHADICTLSEKERQIKKQTALVELVKHKPKAT |
| | | | KEQLKAVMDDFAAFVEKCCKADDKETCFAEEGKKLV |
| | | | AASQAALGL |

What is claimed is:

1. A fusion protein, comprising:
an N-terminal human serum albumin (HSA) polypeptide coupled to an insulin-like growth factor 2 (IGF2) polypeptide comprising an amino acid sequence at least 90% identical to SEQ ID NO: 76.

2. (canceled)

3. The fusion protein of claim 1, wherein the N-terminal HSA polypeptide comprises the amino acid sequence of SEQ ID NO: 109.

4. The fusion protein of claim 1, wherein the IGF2 polypeptide consists of an amino acid sequence that is at least 95% identical to SEQ ID NO: 76.

5. (canceled)

6. The fusion protein of claim 1, wherein the IGF2 polypeptide consists of an amino acid sequence that is at least 98% identical to SEQ ID NO: 76.

7. (canceled)

8. (canceled)

9. The fusion protein of claim 1, wherein a peptide linker separates the N-terminal HSA polypeptide and the IGF2 polypeptide in the fusion protein.

10. (canceled)

11. (canceled)

12. (canceled)

13. The fusion protein of claim 9, wherein the peptide linker comprises a glycine-rich linker.

14. The fusion protein of claim 13, wherein the glycine-rich linker comprises a glycine-serine linker.

15. The fusion protein of claim 14, wherein the glycine-serine linker comprises a multimer of the sequence GGGGS(n), wherein n is 1-5.

16. The fusion protein of claim 14, wherein the glycine-serine linker comprises the amino acid sequence of SEQ ID NO: 104.

17. (canceled)

18. (canceled)

19. (canceled)

20. A pharmaceutical composition comprising the fusion protein of claim 1 and a stabilizing agent.

21. The pharmaceutical composition of claim 20, wherein the stabilizing agent comprises sucrose.

22. A pharmaceutical composition comprising the fusion protein of claim 1 and a non-ionic surfactant.

23. The pharmaceutical composition of claim 22, wherein the non-ionic surfactant comprises a polyethylene glycol compound.

24. A nucleic acid encoding the fusion protein of claim 1.

25. A vector comprising the nucleic acid of claim 24.

26. A method of producing the fusion protein of claim 1, wherein the vector of claim 25 is expressed in a bacterial cell.

27. A method of treating a subject in need thereof, the method comprising administering to the subject the fusion protein of claim 1.

28. The method of claim 27, wherein the subject has an aging disorder, a muscle injury, an injury to a connective tissue, or an injury to a non-muscle soft-tissue, or any combination thereof.

29. A method of treating muscle atrophy in a subject, the method comprising administering to the subject a therapeutically effective amount of an Insulin-like growth factor 2 (IGF2) fusion polypeptide comprising an IGF2 amino acid sequence and an N-terminal human serum albumin (HSA) heterologous polypeptide amino acid sequence, wherein the administering i) increases muscle fiber size, ii) increases muscle fiber number, or iii) increases muscle fiber size and muscle fiber number in the subject.

30. A method of treating sarcopenia in a subject in need thereof, the method comprising administering to the subject a therapeutically effective amount of an Insulin-like growth factor 2 (IGF2) fusion polypeptide comprising an IGF2 amino acid sequence and an N-terminal human serum albumin (HSA) heterologous polypeptide amino acid sequence, wherein the administering slows or reverses a degenerative loss of skeletal muscle mass quality and strength associated with aging in the subject.
